SEQUENCE LISTING (26 sequences)

SEQ ID NO: 1 - protein, 11 aa, Artificial Sequence
  self-assembling peptide P11-4
QQRFEWEFEQQ

SEQ ID NO: 2 - protein, 11 aa, Artificial Sequence
  self-assembling peptide
  MOD_RES (3)..(3): Orn
  MOD_RES (5)..(5): Orn
  MOD_RES (7)..(7): Orn
QQXFXWXFQQQ

SEQ ID NO: 3 - protein, 11 aa, Artificial Sequence
  self-assembling peptide P11-8
  MOD_RES (5)..(5): Orn
  MOD_RES (7)..(7): Orn
QQRFXWXFEQQ

SEQ ID NO: 4 - protein, 11 aa, Artificial Sequence
  self-assembling peptide
QQRFQWQFEQQ

SEQ ID NO: 5 - protein, 11 aa, Artificial Sequence
  self-assembling peptide
QQEFEWEFEQQ

SEQ ID NO: 6 - protein, 10 aa, Artificial Sequence
  self-assembling peptide
  MOD_RES (3)..(3): Orn
  MOD_RES (5)..(5): Orn
  MOD_RES (7)..(7): Orn
  MOD_RES (9)..(9): Orn
QQXFXWXFXQ

SEQ ID NO: 7 - protein, 11 aa, Artificial Sequence
  self-assembling peptide
EQEFEWEFEQE

SEQ ID NO: 8 - protein, 11 aa, Artificial Sequence
  self-assembling peptide
QQEFEWEFEQQ

SEQ ID NO: 9 - protein, 11 aa, Artificial Sequence
  self-assembling peptide
ESEFEWEFESE

SEQ ID NO: 10 - protein, 11 aa, Artificial Sequence
  self-assembling peptide
  MOD_RES (3)..(3): Orn
  MOD_RES (5)..(5): Orn
  MOD_RES (7)..(7): Orn
  MOD_RES (9)..(9): Orn
QQXFXWXFXQQ

SEQ ID NO: 11 - protein, 11 aa, Artificial Sequence
  self-assembling peptide
  MOD_RES (1)..(1): Orn
  MOD_RES (3)..(3): Orn
  MOD_RES (5)..(5): Orn
  MOD_RES (7)..(7): Orn
  MOD_RES (9)..(9): Orn
  MOD_RES (11)..(11): Orn
XQXFXWXFXQX

SEQ ID NO: 12 - protein, 11 aa, Artificial Sequence
  self-assembling peptide
  MOD_RES (3)..(3): Orn
  MOD_RES (5)..(5): Orn
  MOD_RES (7)..(7): Orn
  MOD_RES (9)..(9): Orn
SSXFXWXFXSS

SEQ ID NO: 13 - protein, 11 aa, Artificial Sequence
  self-assembling peptide
SSRFEWEFESS

SEQ ID NO: 14 - protein, 11 aa, Artificial Sequence
  self-assembling peptide
  MOD_RES (5)..(5): Orn
  MOD_RES (7)..(7): Orn
SSRFXWXFESS

SEQ ID NO: 15 - protein, 11 aa, Artificial Sequence
  self-assembling peptide
  MOD_RES (3)..(3): Orn
  MOD_RES (5)..(5): Orn
  MOD_RES (7)..(7): Orn
  MOD_RES (9)..(9): Orn
QQXFXWXFXQQ

SEQ ID NO: 16 - protein, 11 aa, Artificial Sequence
  self-assembling peptide
NNRFEWEFENN

SEQ ID NO: 17 - protein, 11 aa, Artificial Sequence
  self-assembling peptide
  MOD_RES (5)..(5): Orn
  MOD_RES (7)..(7): Orn
NNRFXWXFENN

SEQ ID NO: 18 - protein, 11 aa, Artificial Sequence
  self-assembling peptide
TTRFEWEFETT

SEQ ID NO: 19 - protein, 11 aa, Artificial Sequence
  self-assembling peptide
  MOD_RES (5)..(5): Orn
  MOD_RES (7)..(7): Orn
TTRFXWXFETT

SEQ ID NO: 20 - protein, 11 aa, Artificial Sequence
  self-assembling peptide P11-20
QQRQEQEQEQQ

SEQ ID NO: 21 - protein, 5 aa, Artificial Sequence
  designed self-assembling peptide, consensus sequence 1
  VARIANT 1..1: amino acid with acidic or basic side chain
  VARIANT 2..2: amino acid with hydrophobic or polar side chain that is selected from the group consisting of alanine, valine, isoleucine, leucine, methionine, phenylalanine, tyrosine, tryptophan and glutamine
  VARIANT 3..3: amino acid with acidic or basic side chain
  VARIANT 4..4: amino acid with hydrophobic or polar side chain that is selected from the group consisting of alanine, valine, isoleucine, leucine, methionine, phenylalanine, tyrosine, tryptophan and glutamine
  VARIANT 5..5: amino acid with acidic or basic side chain
XXXXX

SEQ ID NO: 22 - protein, 5 aa, Artificial Sequence
  designed self-assembling peptide assembling at a pH below 7.5
  VARIANT 1..1: amino acid with acidic side chain
  VARIANT 2..2: amino acid with hydrophobic or polar side chain that is selected from the group consisting of alanine, valine, isoleucine, leucine, methionine, phenylalanine, tyrosine, tryptophan and glutamine
  VARIANT 3..3: amino acid with acidic side chain
  VARIANT 4..4: amino acid with hydrophobic or polar side chain that is selected from the group consisting of alanine, valine, isoleucine, leucine, methionine, phenylalanine, tyrosine, tryptophan and glutamine
  VARIANT 5..5: amino acid with acidic side chain
XXXXX

SEQ ID NO: 23 - protein, 5 aa, Artificial Sequence
  designed self-assembling peptide
  VARIANT 1..1: amino acid with acidic side chain
  VARIANT 2..2: amino acid with hydrophobic side chain that is selected from the group consisting of alanine, valine, isoleucine, leucine, methionine, phenylalanine, tyrosine and tryptophan
  VARIANT 3..3: amino acid with acidic side chain
  VARIANT 4..4: amino acid with hydrophobic side chain that is selected from the group consisting of alanine, valine, isoleucine, leucine, methionine, phenylalanine, tyrosine and tryptophan
  VARIANT 5..5: amino acid with acidic side chain
XXXXX

SEQ ID NO: 24 - protein, 5 aa, Artificial Sequence
  designed self-assembling peptide
  VARIANT 2..2: amino acid with hydrophobic side chain that is selected from the group consisting of alanine, valine, isoleucine, leucine, methionine, phenylalanine, tyrosine and tryptophan
  VARIANT 4..4: amino acid with hydrophobic side chain that is selected from the group consisting of alanine, valine, isoleucine, leucine, methionine, phenylalanine, tyrosine and tryptophan
EXEXE

SEQ ID NO: 25 - protein, 5 aa, Artificial Sequence
  designed self-assembling peptide
  VARIANT 2..2: amino acid with hydrophobic side chain that is selected from the group consisting of alanine, valine, isoleucine, leucine, methionine, phenylalanine, tyrosine and tryptophan
  VARIANT 4..4: amino acid with hydrophobic side chain that is selected from the group consisting of alanine, valine, isoleucine, leucine, methionine, phenylalanine, tyrosine and tryptophan
DXDXD

SEQ ID NO: 26 - protein, 5 aa, Artificial Sequence
  designed self-assembling peptide
EQEQE

(19) United States
(12) Patent Application Publication
(10) Pub. No.: US 2023/0203590 A1
(43) Pub. Date: Jun. 29, 2023

(54) METHODS AND MEANS FOR DIAGNOSING LUNG CANCER

(71) Applicants: Forschungszentrum Borstel, Borstel (DE); Christian-Albrechts-Universität zu Kiel, Kiel (DE); LungenClinic Grosshansdorf, Grosshansdorf (DE)

(72) Inventors: Torsten GOLDMANN, Reinfeld (DE); Sebastian MARWITZ, Reinfeld (DE); Ole AMMERPOHL, Munderkingen (DE); Swetlana SCHEUFELE, Kiel (DE); Martin RECK, Ahrensburg (DE)

(21) Appl. No.: 17/639,804

(22) PCT Filed: Sep. 4, 2020
(86) PCT No.: PCT/EP2020/074775

§ 371 Date: Jul. 5, 2022

(30) Foreign Application Priority Data
Sep. 5, 2019 (EP) 19195688.7

(51) Int. Cl. C12Q 1/6886; C12Q 1/6869

(52) U.S. Cl. CPC C12Q 1/6886; C12Q 1/6869; C12Q 2600/112; C12Q 2600/118; C12Q 2600/154

(57) ABSTRACT

The present invention relates to the diagnosis of lung tumors. It provides methods suitable both for diagnosing lung tumors on the basis of surgical samples and lung biopsies (here, e.g., with the aid of DNA microarrays) and of liquid biopsies. In the case of liquid biopsies, cell-free DNA (cfDNA) is used. In this context, both particularly suitable analysis methods and particularly suitable sets of methylation markers are described. Means suitable for diagnosing lung cancer by examinining the methylation of a set of methylation markers, e.g., in cell-free DNA (cfDNA) from liquid biopsy samples of patients, wherein the means comprises oligonucleotides which can hybridize to DNA comprising the methylation markers, as well as the use of said methods and means for diagnosing, i.e., e.g., determinination, subtyping and prognostic characterization of lung tumors are also an object of the invention.

The present invention relates to the diagnosis of lung tumors. It provides methods suitable both for diagnosing lung tumors on the basis of surgical samples and lung biopsies (here, e.g., with the aid of DNA microarrays) and of liquid biopsies. In the case of liquid biopsies, cell-free DNA (cfDNA) is used. In this context, both particularly suitable analysis methods and particularly suitable sets of methylation markers are described. Means suitable for diagnosing lung cancer by examining the methylation of a set of methylation markers, e.g., in cell-free DNA (cfDNA) from liquid biopsy samples of patients, wherein the means comprises oligonucleotides which can hybridize to DNA comprising the methylation markers, as well as the use of said methods and means for diagnosing, i.e., e.g., determinination, subtyping and prognostic characterization of lung tumors, are also objects of the invention.

Lung cancer is the second most common type of cancer in men and women worldwide. In Germany, approx. 52,500 new cases are registered annually. The mean age of onset of disease is 70 years for men and 69 years for women. A distinction is made between small cell lung cancer (SCLC) and non-small cell lung cancer (NSCLC). NSCLCs are distinctly more common and occur in 85% of the affected patients. Furthermore, several subentities are distinguished in the case of NSCLCs, of which the most common are adenocarcinoma and squamous cell carcinoma.

The fact that the disease symptoms usually occur very late is reflected in a poor prognosis. The 5-year survival rate is at 15%.

Like most other tumors, lung carcinomas exhibit high genomic heterogeneity. For example, mutations within KRAS, EGFR, BRAF, MEK1, MET, HER2, ALK, ROS1, RET, FGFR1, DDR2, PTEN, LKB1, RB1, CDKN2A or TP53 genes can induce the development of a primary lung carcinoma. In addition, so-called passenger mutations accumulate during the course of tumor evolution, which can lead to various subclones. This fact renders the development of a reliable early-detection test based only on molecular-genetic mutation analyses very difficult, which becomes apparent from many examples in the literature.

For example, Uchida et al. have carried out a lung carcinoma screening based on typical mutations of the EGFR gene. The average sensitivity of this test was only 54.4% and dropped to 22.2% in the case of early stages IA-IIIA (Uchida et al. [2015] Clin. Chem. 61: 1191-1196). Couraud et al. developed an NGS-based test, in which the best-known mutations within the EGFR, BRAF, KRAS, HER2 and PIK3CA genes were analyzed in plasma. The sensitivity of said test was 58%. Here too, the detection of tumors in early stages posed a problem (Couraud et al. [2014] Clin. Cancer Res. 20: 4613-4624). In 2014, Newmann et al. developed the CAPP-Seq.

This was an optimized NGS protocol with an associated bioinformatic evaluation pipeline. In the case of CAPP-Seq, the best-known NSCLC mutations in plasma are sequenced and analyzed, which allowed for identifying 100% of stage II to IV lung cancer patients. However, the identification of tumors in stage I again posed a problem here, and the corresponding sensitivity was only 50% (Newman et al. [2014] Nat. Methods 20: 548-554). These examples clearly show the problem in developing a reliable early-detection test for lung carcinoma that is based only on genomic analyses.

In addition to mutations, epimutations also play a decisive role during tumor evolution. For example, promoters within certain tumor suppressor genes become hypermethylated, which, in turn, results in their transcriptional repression. This phenomenon is accompanied by the overexpression of DNA methyltransferases. Promoter hypermethylation has been described particularly frequently In the literature within the P16INK4A, RASSF1A, APC, RARB, CDH1, CDH13, DAPK, FHIT and MGMT genes (Langevin et al. [2015] Transl. Res. 165: 74-90).

The genome-wide hypomethylation of NSCLC is associated with genomic instability. Targeted hypomethylation of genes has so far been identified only in the case of MAGEA3/6, TKTL1, BORIS, DDR1, YWHAZ and TMSB10 (inter alia, Newman et al. [2014] Nat. Methods 20: 548-554).

Furthermore, malignant lung tumors frequently exhibit altered histone acetylation at positions H4K5, H4K8, H4K12 and H4K16. The global proportion of H4K20me3, too, is lower in NSCLC than in healthy lung tissue (Newman et al. [2014] Nat. Methods 20: 548-554). In addition, aberrant ncRNA expression can occur, such as, e.g., MIR196A, MIR200B, MALAT1 and HOTAIR.

According to national and international recommendations, the affected patients are currently initially subjected to a comprehensive physical examination in the event of a suspected diagnosis. Subsequently, the thorax is examined by imaging methods such as, e.g., radiography or computed tomography (CT). If tumors are detected in this process, subsequent bronchoscopies are recommended, during which the lungs are thoroughly analyzed endoscopically and biopsies of the tumors are taken. Said biopsies are, then, subjected to histological, immunohistochemical and molecular-genetic analyses.

During the histological examinations, it is determined whether the tumors are malignant. If this is the case, their entity is ascertained. To identify the optimal therapy, molecular-genetic and imaging methods are additionally considered. Due to the radiation exposure and invasiveness, especially the imaging and endoscopic methods can be stressful here for the affected patients.

The detection limit of the radiological methods is at a tumor size of 7 to 10 mm, which corresponds to cell clusters consisting of already roughly one billion tumor cells. An alternative, less invasive method is based on liquid biopsies, by means of which tumors can be detected much earlier, from a size of ca. 50 million cells.

In case of liquid biopsies, a few milliliters of blood are collected from the patient. Circulating cell-free DNA (cfDNA) can then be isolated from the blood plasma or blood serum. In the human body, cfDNA is formed during apoptotic and necrotic processes. This involves the cleavage of cellular, genomic DNA (gDNA) by DNAses into fragments of ca. 167 bp in length and their release into the bloodstream.

In the case of patients suffering from malignant diseases, the total amount of cfDNA additionally contains tumor DNA. The amount of cfDNA can vary greatly depending on the entity or stage of the disease. However, it contains diagnostically, therapeutically and prognostically relevant information.

In addition to genetic mutations of a tumor, epimutations can also be analyzed. In this context, DNA methylation is of particular interest. The DNA methylation pattern is tissue-specific and already changes in early phases of tumor evolution. Furthermore, a study of the GNAS1 locus made clear that cfDNA methylation in the blood remains stable. It is neither modified nor distorted and is thus suitable as a biomarker in clinical diagnostics (Puszyk et al. [2009] Clin. Chim. Acta 400: 107-110).

The diagnostic potential of DNA methylation has already been made clear by several studies. For instance, a SOX17 study in stomach carcinoma showed that the overall survival of the patient cohorts correlated with the detected amount of methylated SOX17 cfDNA (Balgkouranidou et al. [2013] Clin. Chem. Lab. Med. 51: 1505-1510). A study with female patients suffering from breast carcinoma showed significant hypermethylation of the CST6 gene (Chimonidou et al. [2013] Clin. Biochem. 46: 235-240). Liggett et al. were able to distinguish between pancreatic carcinoma and its precursor, chronic pancreatitis, based on the DNA methylation pattern (Liggett et al. [2010] Cancer 116: 1674-1680).

Alterations in the DNA methylation pattern have also been described in NSCLC by several working groups. For example, Balgkouranidou et al. could detect significant hypermethylation of the BRMS1 gene in patients with bronchial carcinoma (Balgkouranidou et al. [2014] Brit. J. Cancer 110: 2054-2062). In 2016, Marwitz et al. detected DNA hypomethylation within the CTLA4 and PDCD1 genes. Said genes were overexpressed at the transcriptome level. Since these are therapeutically important checkpoint regulators, this work is of great therapeutic relevance (Marwitz et al. [2017] Clin. Epigenet. 9: 51).

The diagnostic potential of DNA methylation also becomes clear from the example of the “Epi proLung” assay (“Epigenomics AG”, Germany). In this case, the cfDNA methylation pattern of the SHOX2 and PTGER4 genes is analyzed. At a specificity of 90%, the sensitivity is 67% (Weiss et al. [2017] J. Thorac. Oncol. 12: 77-84). Therefore, the sensitivity of the “Epi proLung” test is insufficient for reliable lung cancer screening. As yet, there are no further liquid biopsy-based methods which allow reliable, preventive early detection of lung cancer.

In comparison, the inventors addressed the problem of providing a more reliable method for diagnosing lung cancer. This problem is solved by the invention, especially by the subject matter of the claims.

One aspect of the invention is a method for diagnosing lung cancer, wherein the methylation of a set of methylation markers in a sample of a patient is determined, wherein, e.g., cfDNA from a liquid biopsy can be examined. Alternatively, the sample can also be a tissue sample, e.g., a solid tissue sample from a tumor or from a tissue in which a tumor is possibly present. In particular, the tissue sample can originate from a biopsy or surgical material of lung tissue. Pleural fluid can be examined, too. The method according to the invention is distinguished by the fact that, owing to the selection of markers, it is particularly well suited to being used for examination of tissue samples taken during surgery, for examination of lung biopsy tissue and for examination of cfDNA from a liquid biopsy. In the context of the invention, surgeries in which tissue is collected as a sample will usually be surgeries for removal of a diagnosed lung tumor. Even then, however, questions will still arise, which the method according to the invention can answer, for instance about the entity and/or prognosis of the tumor or in relation to the demarcation between tumor tissue and adjacent normal tissue.

The invention provides a method for diagnosing lung cancer, wherein the methylation of a set of methylation markers, e.g., in cfDNA from a liquid biopsy sample of a patient, is determined, wherein, optionally, an alignment against a reference genome using the Segemehl algorithm is carried out.

The invention further provides a method for diagnosing lung cancer, wherein the methylation of a set of methylation markers, e.g., in cfDNA from a liquid biopsy sample of a patient, is determined, wherein, optionally, the methylation of methylation markers in the genes SERPINB5, DOCK10, PCDHB2, HIF3A, FGD5, RCAN2, HOXD12, OCA2, SLC22A20, FADL-1, NRXN1, ACOXL, FAM53A, UBE3D and AUTS2 is determined.

For minimally invasive diagnostics of lung tumors (lung carcinomas), according to the invention, use is made of, e.g., the circulating cell-free DNA (cfDNA) from liquid biopsies, e.g., from plasma, blood or serum, preferably from plasma. If a patient is suffering from a malignant tumor disease, the total amount of circulating DNA also contains the tumor DNA, which contains all therapeutically and prognostically relevant information about the genetic and epigenetic characteristics of the tumor. The invention provides both preferred methods for diagnosing lung cancer on this basis and preferred sets of methylation markers.

In the context of the invention, it was shown that the methylation signatures in solid tumors, e.g., in samples from surgeries or biopsies, partly differ from the signatures from cfDNA from liquid biopsies. This can explain why the abovementioned “Epi proLung” study, in which the cfDNA methylation profile within the SHOX2 and PTGER4 genes was analyzed, exhibited, at a specificity of 90%, only a sensitivity of 67% (Weiss et al. [2017] J. Thorac. Oncol. 12: 77-84). The SHOX2 and PTGER4 biomarkers used originate from analyses of primary tumor tissue (Murn et al. [2008] J. Exp. Med. 205: 3091-3103; and Schneider et al. [2011] BMC Cancer 11: 102). However, the present invention clearly shows (see section 2.1.3) that the DNA methylation patterns correlate only to a limited extent between the cfDNA from the plasma and the gDNA from a primary tumor. Indeed, the total amount of cfDNA contains not only DNA derived from the lung or a tumor, but also DNA from further tissues and organs.

This means that the strongly aberrant methylated DNA regions in the primary tumor tissue do not necessarily exhibit differential methylation in the plasma. Therefore, it is not sufficient for the development of a noninvasive, cfDNA-based early-detection test to use known biomarkers from the primary tumors. Instead, it is necessary to identify novel cfDNA-specific, strong and unambiguous methylation signatures in the plasma of the affected patients. However, cfDNA-specific methylation signatures are in return also not necessarily suitable for diagnosis and examination of tissue samples. Therefore, the goal was - in distinction to the approaches known in the state of the art - to determine universal methylation signatures, by means of which very different (also complex) patient samples (also with greatly varying content of tumor cells) can be examined robustly and reliably. This was achieved using the present invention. According to the invention, it is advantageous that the identified markers provide good results both with tissue samples, e.g., solid tissue samples from tumor tissue, and with liquid biopsies and are thus suitable for diagnosing lung cancer from various types of samples.

To identify a set of methylation markers according to the invention that comprises particularly informative differentially methylated regions, multiple steps were carried out in the context of the invention, which are described in detail in the Example section. First, DNA methylation signatures were examined in 40 malignant lung tumors and their corresponding controls. DNA methylation signatures were then analyzed in the blood plasma of nine patients. Of these, five patients were suffering from adenocarcinoma of the lungs and four from squamous cell carcinoma of the lung. By contrast, the remaining patients were free of malignant diseases and formed the control cohorts. Finally, additional data sets from multiple studies that have been made available were evaluated, which made it possible to identify further tumor-specific and prognostic CpG loci. The set of methylation markers synthesized on this basis, also referred to as plasma panel (see Table 1), was subsequently validated in the context of a pilot study. Said set of methylation markers comprises a plurality of regions which, e.g., are differentially methylated in cfDNA and, surprisingly, allow for a specific statement about the presence of a tumor, the tumor entity, the tumor stage and/or the prognosis.

In one embodiment, the invention therefore relates to a method for diagnosing lung cancer, in which the methylation of a set of methylation markers in a sample of the patient is determined, wherein the set of methylation markers is selected from the group consisting of the regions listed in Tables 1a, 1b and 1c and comprises at least 60 regions, preferably at least 64 regions, more preferably at least 340 or at least 350 regions, most preferably at least 630 regions. For example, methylation markers can be determined to determine the presence of a tumor.

The invention also relates to a method for diagnosing lung cancer, in which the methylation of a set of methylation markers in a sample of the patient is determined, wherein the set of methylation markers is selected from the group consisting of the regions listed in Tables 1a, 1b and 1c and comprises at least 134 regions, preferably 138 regions, more preferably at least 240 regions, most preferably at least 247 regions. For example, methylation markers can be determined to determine the entity of a tumor.

According to the invention, the set of methylation markers can comprise at least 194 regions, preferably at least 600 regions, optionally all 630 regions. For example, at least 60, preferably at least 64 methylation markers can be determined to determine the presence of a tumor, e.g., methylation markers from Table 1a, and at least 134, preferably 138 methylation markers can be determined to determine the entity of the tumor, e.g., methylation markers from Table 1b. The more methylation markers are determined, the more accurate the analysis. Therefore, at least 150, preferably at least 340 or even 350 methylation markers can also be determined to determine the presence of a tumor, e.g., methylation markers from Table 1a, and at least 240 or even 247 methylation markers can be determined to determine the entity of the tumor, e.g., methylation markers from Table 1b. Optionally, at least 15, preferably at least 30 or even 33 methylation markers from Table 1c can be additionally determined to determine the prognosis.

In one embodiment, the invention therefore relates to a method for diagnosing lung cancer, in which the methylation of a set of methylation markers in a sample of a patient, e.g., in cfDNA from a liquid biopsy sample of a patient, is determined, wherein the set of methylation markers comprises at least 60 regions selected from the group consisting of:

| Chromosome | Start | End |
|---|---|---|
| chr1 | 6165201 | 6165361 |
| chr1 | 17567892 | 17568189 |
| chr1 | 15426262 | 15426418 |
| chr1 | 15670403 | 15670539 |
| chr2 | 1126410 | 1126557 |
| chr2 | 225642009 | 225642217 |
| chr2 | 236745514 | 236745688 |
| chr2 | 240881986 | 240882138 |
| chr2 | 2179742 | 2179886 |
| chr2 | 30747398 | 30747539 |
| chr2 | 175998270 | 175998415 |
| chr2 | 219647407 | 219647560 |
| chr3 | 56445240 | 56445378 |
| chr3 | 85143433 | 85143600 |
| chr3 | 146123966 | 146124095 |
| chr3 | 68947379 | 68947542 |
| chr3 | 197767819 | 197767978 |
| chr4 | 143487129 | 143487273 |
| chr4 | 26398190 | 26398329 |
| chr4 | 77647893 | 77648027 |
| chr4 | 102497551 | 102497732 |
| chr5 | 39187156 | 39187287 |
| chr5 | 56145736 | 56145896 |
| chr5 | 160171748 | 160171896 |
| chr5 | 16793080 | 16793219 |
| chr5 | 76869108 | 76869253 |
| chr6 | 169050287 | 169050447 |
| chr6 | 76773251 | 76773422 |
| chr6 | 123869831 | 123869971 |
| chr7 | 6268960 | 6269087 |
| chr7 | 38508407 | 38508486 |
| chr7 | 153743779 | 153743947 |
| chr7 | 137230794 | 137230963 |
| chr7 | 151300131 | 151300282 |
| chr8 | 3672236 | 3672387 |
| chr8 | 99510084 | 99510252 |
| chr8 | 101170822 | 101170975 |
| chr8 | 141127042 | 141127183 |
| chr9 | 2050654 | 2050804 |
| chr9 | 9227683 | 9227824 |
| chr9 | 79060522 | 79060633 |
| chr9 | 124334690 | 124334848 |
| chr9 | 126166694 | 126166828 |
| chr10 | 96279972 | 96280055 |
| chr10 | 97033594 | 97033733 |
| chr11 | 134245966 | 134246129 |
| chr12 | 8004422 | 8004573 |
| chr12 | 97140774 | 97140905 |
| chr12 | 111566555 | 111566698 |
| chr12 | 117750775 | 117750937 |
| chr13 | 36828740 | 36828902 |
| chr14 | 93214072 | 93214242 |
| chr15 | 56006471 | 56006552 |
| chr15 | 101547384 | 101547527 |
| chr16 | 4141795 | 4141956 |
| chr18 | 21857621 | 21857750 |
| chr18 | 29528340 | 29528468 |
| chr18 | 46845901 | 46846043 |
| chr19 | 874766 | 874934 |
| chr19 | 6799968 | 6800095 |
| chr20 | 20243607 | 20243747 |
| chr20 | 55079800 | 55079945 |
| chr21 | 30502729 | 30502871 |
| chr21 | 46587906 | 46588052 |

The aforementioned methylation markers are the markers mentioned in Table 1a,which were identified only in cfDNA. In this analysis, the presence of a tumor is preferably examined, wherein the set of methylation markers optionally comprises all the regions of the group.

In this context, the set of methylation markers can comprise at least 340 regions selected from the group consisting of the regions listed in Table 1a, wherein the set of methylation markers preferably comprises all the regions listed in Table 1a.

In one embodiment of the abovementioned methods, the set of methylation markers comprises at least 134 regions selected from the group consisting of

| Chromosome | Start | End |
|---|---|---|
| chr1 | 3289010 | 3289139 |
| chr1 | 17567892 | 17568189 |
| chr1 | 23284417 | 23284507 |
| chr1 | 24277975 | 24278154 |
| chr1 | 47738990 | 47739142 |
| chr1 | 79467955 | 79468081 |
| chr1 | 108975333 | 108975476 |
| chr1 | 196682870 | 196683025 |
| chr1 | 217310510 | 217310654 |
| chr1 | 240656480 | 240656649 |
| chr1 | 240746545 | 240746706 |
| chr1 | 246241918 | 246242056 |
| chr2 | 1129413 | 1129596 |
| chr2 | 1334513 | 1334640 |
| chr2 | 23917010 | 23917136 |
| chr2 | 25124037 | 25124165 |
| chr2 | 46779214 | 46779381 |
| chr2 | 113534514 | 113534653 |
| chr2 | 120417931 | 120418073 |
| chr2 | 131798797 | 131798977 |
| chr2 | 198073787 | 198073950 |
| chr2 | 205889570 | 205889704 |
| chr2 | 207319476 | 207319691 |
| chr3 | 3755582 | 3755730 |
| chr3 | 14959981 | 14960128 |
| chr3 | 25581721 | 25581859 |
| chr3 | 75834579 | 75834736 |
| chr3 | 87031909 | 87032079 |
| chr3 | 122710736 | 122710872 |
| chr3 | 139727561 | 139727706 |
| chr3 | 145864433 | 145864574 |
| chr4 | 1665996 | 1666155 |
| chr4 | 22518120 | 22518271 |
| chr4 | 77306769 | 77306948 |
| chr4 | 82520036 | 82520212 |
| chr4 | 155413871 | 155414011 |
| chr4 | 156601279 | 156601436 |
| chr4 | 162457724 | 162457860 |
| chr4 | 176636441 | 176636580 |
| chr4 | 177654193 | 177654363 |
| chr5 | 14450118 | 14450272 |
| chr5 | 75935318 | 75935450 |
| chr5 | 140475728 | 140475872 |
| chr5 | 146345906 | 146346062 |
| chr5 | 156458027 | 156458167 |
| chr5 | 157169890 | 157170038 |
| chr6 | 20832000 | 20832349 |
| chr6 | 24420281 | 24420413 |
| chr6 | 36331071 | 36331215 |
| chr6 | 54074847 | 54075021 |
| chr6 | 71122323 | 71122483 |
| chr6 | 83604672 | 83604779 |
| chr6 | 90709859 | 90710016 |
| chr6 | 111744738 | 111744881 |
| chr6 | 148806765 | 148806922 |
| chr6 | 155574119 | 155574263 |
| chr6 | 158460178 | 158460323 |
| chr7 | 5549605 | 5549675 |
| chr7 | 40669616 | 40669796 |
| chr7 | 73799798 | 73799908 |
| chr7 | 78030021 | 78030155 |
| chr7 | 81399230 | 81399365 |
| chr7 | 134452355 | 134452524 |
| chr7 | 140335200 | 140335344 |
| chr7 | 146925646 | 146925824 |
| chr7 | 153976496 | 153976643 |
| chr7 | 157941162 | 157941344 |
| chr7 | 157980130 | 157980264 |
| chr7 | 157980485 | 157980624 |
| chr7 | 158314155 | 158314301 |
| chr8 | 6392188 | 6392336 |
| chr8 | 11724061 | 11724159 |
| chr8 | 17237496 | 17237639 |
| chr8 | 21803649 | 21803801 |
| chr8 | 52696850 | 52697008 |
| chr8 | 72183950 | 72184120 |
| chr8 | 81042553 | 81042694 |
| chr8 | 85101824 | 85101952 |
| chr8 | 110703169 | 110703320 |
| chr8 | 121727803 | 121727944 |
| chr8 | 133476418 | 133476558 |
| chr9 | 8813022 | 8813150 |
| chr9 | 90258110 | 90258253 |
| chr9 | 97061691 | 97061835 |
| chr10 | 12533631 | 12533768 |
| chr10 | 32647546 | 32647656 |
| chr10 | 32657588 | 32657719 |
| chr10 | 37511104 | 37511239 |
| chr10 | 62708104 | 62708269 |
| chr10 | 73207931 | 73208064 |
| chr10 | 108812804 | 108812940 |
| chr10 | 115658133 | 115658275 |
| chr10 | 123914649 | 123914808 |
| chr11 | 15025357 | 15025499 |
| chr11 | 19778770 | 19778909 |
| chr11 | 26355535 | 26355711 |
| chr11 | 26600784 | 26600925 |
| chr11 | 26626367 | 26626558 |
| chr11 | 41275397 | 41275536 |
| chr11 | 62158845 | 62158985 |
| chr11 | 70503001 | 70503139 |
| chr11 | 106592142 | 106592304 |
| chr11 | 120644150 | 120644282 |
| chr11 | 122678508 | 122678636 |
| chr11 | 128851150 | 128851286 |
| chr12 | 125571801 | 125571933 |
| chr13 | 48806444 | 48806588 |
| chr13 | 113527733 | 113527876 |
| chr14 | 35030336 | 35030470 |
| chr14 | 104486171 | 104486314 |
| chr15 | 22839905 | 22840043 |
| chr15 | 26964926 | 26965065 |
| chr15 | 29246303 | 29246447 |
| chr15 | 30180680 | 30180842 |
| chr15 | 32404970 | 32405130 |
| chr15 | 64244033 | 64244215 |
| chr15 | 68530927 | 68531091 |
| chr15 | 83579367 | 83579513 |
| chr15 | 88559865 | 88560003 |
| chr16 | 6257325 | 6257474 |
| chr16 | 15665564 | 15665721 |
| chr16 | 24321180 | 24321320 |
| chr16 | 75528556 | 75528698 |
| chr16 | 88013993 | 88014135 |
| chr16 | 89713952 | 89714124 |
| chr17 | 416719 | 416865 |
| chr17 | 19809670 | 19809830 |
| chr17 | 21086965 | 21087112 |
| chr17 | 33364961 | 33365040 |
| chr17 | 64330485 | 64330837 |
| chr17 | 75142732 | 75142885 |
| chr19 | 11890923 | 11891074 |
| chr19 | 49016450 | 49016584 |
| chr19 | 57922060 | 57922195 |
| chr20 | 9706282 | 9706429 |
| chr20 | 33713618 | 33713757 |
| chr21 | 33340955 | 33341038 |
| chr22 | 21206849 | 21206995 |
| chr22 | 30292326 | 30292475 |
| chr22 | 35697444 | 35697606 |

The aforementioned methylation markers are the markers mentioned in Table 1b,which were identified only in cfDNA. In this analysis, the entity of a tumor is preferably examined, wherein, in particular, a distinction can be made between adenocarcinoma and squamous cell carcinoma. In this context, the set of methylation markers can comprise all regions of the group.

In this analysis, the set of methylation markers can also comprise at least 240 regions, wherein the group consists of the regions listed in Table 1b. Preferably, the set of methylation markers comprises all regions of the group listed in Table 1b.

Since it has been shown that all the regions defined in Tables 1a and 1b are differentially methylated in the samples examined, it is advantageous to analyze all regions defined in Tables 1a and 1b, especially if both the presence and the entity of a potential tumor are to be analyzed.

The validity of the analysis is greatest if the set of methylation markers comprises at least 620 regions from a group consisting of all regions listed in Table 1, especially if the prognosis is further determined, preferably if the set of methylation markers comprises allregions of the group.

During further analysis of the data and verification on the basis of cfDNA from patients, a second set of methylation markers having various subgroups was identified in the context of the invention, by means of which different questions can be answered (see Tables 2-4). The corresponding methylation markers are defined differentially methylated positions which lie in the regions mentioned in Table 1. The methylation markers mentioned in Tables 2-4 thus represent suitable subgroups for examination of the methylation markers contained in the plasma panel.

Thus, in the context of the invention, either differentially methylated regions, e.g., the regions defined in Tables 1a, 1b, and/or 1c, can serve as methylation markers, or differentially methylated positions. In this regard, the analysis of entire regions leads to more reliable results, since specific positions need not necessarily have the same informative value in the case of particular patients. For this, an analysis of specific positions is possible with less effort, e.g., via an array, and is therefore favorable if a cost-effective diagnosis is to be made. The choice is therefore based on a consideration of the reliability required in the particular case and the possible effort. Evidently, both types of methylation markers can also be used simultaneously for diagnosis. Furthermore, the amount of sample available also plays a role, since especially tissue samples from surgeries contain amounts of DNA sufficient for carrying out an analysis of individual methylated positions via an array.

Particularly informative methylation markers identified in this context lie, in some cases, within the genes SERPINB5, DOCK10, PCDHB2, HIF3A, FGD5, RCAN2, HOXD12, OCA2, SLC22A20, FADL-1, NRXN1, ACOXL, FAM53A, UBE3D and AUTS2. Said genes had hitherto never been specifically described in connection with lung carcinomas or certain NSCLC entities.

The role of some of these genes in tumor evolution and prognosis is known in other cancer types. SERPIN5 is, e.g., a known oncogene (Lei et al. [2011] Oncol. Rep. 26: 1115-1120). HOX genes are aberrantly expressed in many cancer types (Bhatlekar et al. [2014] J. Mol. Med. 92: 811-823). Dysregulation of RCAN2 leads to proliferation of tumor cells (Niitsu et al. [2016] Oncogenesis 5: e253). In some studies, altered expression of DOCK10 had resulted in the migration of melanoma cells (Gadea et al. [2008] Curr. Biol. 18: 1456-1465). Some OCA2 mutations are associated with an increased risk of melanoma, too (Hawkes et al. [2013] J. Dermatol. Sci. 69: 30-37). Furthermore, HIF3A and FGD5 are important angiogenesis regulators and therefore play a crucial role during tumor evolution (Jackson et al. [2010] Expert Opin. Therap. Targets 14: 1047-1057); and Kurogane et al. [2012] Arterioscler. Thromb. Vasc. Biol. 32: 988-996). The DNA methylation of some PCDHB2-CpG loci is associated with a poor prognosis of neuroblastoma patients (Abe et al. [2005] Cancer Res. 65: 828-834). Altered metabolism is, e.g., a characteristic of malignant tumors; in this case, the FADL-1 fatty acid transporter and some SLC transporters may play an important role (Lin et al. [2015] Nat. Rev. Drug Discov. 14: 543-560; and Black [1991] J. Bacteriol. 173: 435-442). UBE3D encodes a ubiquitin protein ligase. Several studies have shown that some ubiquitin protein ligases may play an important role during tumor evolution (see, inter alia, Lisztwan et al. [1999] Genes Dev. 13: 1822-1833). AUTS2 and NRXN1 are neural genes. Overexpression of AUTS2 has been demonstrated in liver metastases (Oksenberg & Ahituv [2013] Trends Genet. 29: 600-608). NRXN1 might be responsible for nicotine addiction (Ching et al. [2010] Am. J. Med. Genet. B. Neuropsychiatr. Genet. 153B: 937-947). Increased expression of ACOXL has already been described in prostate carcinomas (O′Hurley et al. [2015] PLoS One 10: e0133449). Some studies describe FAM53A as a prognostic and therapeutic breast carcinoma marker (Fagerholm et al. [2017] Oncotarget 8: 18381-18398). However, the aforementioned studies do not allow any conclusions that a methylation in these genes, let alone in the positions mentioned in Tables 2-4, correlates with a lung cancer disease and can accordingly be used as a diagnostic marker for the presence of lung tumors or for the establishment of the entity or for the determination of the tumor stage.

Thus, the invention provides, for the first time, a method for diagnosing lung cancer, wherein the methylation of a set of methylation markers, e.g., in cfDNA from a liquid biopsy sample of a patient, is determined, wherein the methylation of methylation markers in the genes SERPINB5, DOCK10, PCDHB2, HIF3A, FGD5, RCAN2, HOXD12, OCA2, SLC22A20, FADL-1, NRXN1, ACOXL, FAM53A, UBE3D and AUTS2 is determined.

Preferably, said methylation markers comprise the methylation markers mentioned in Table 2, especially if the presence of a lung carcinoma is to be determined. Alternatively, especially if the entity of a lung carcinoma is to be determined, and especially if a distinction is to be made between adenocarcinoma and squamous cell carcinoma NSCLC types, the methylation markers comprise the methylation markers mentioned in Table 3. Preferably, both the methylation markers mentioned in Table 2 and those mentioned in Table 3 are determined to answer both questions. Optionally, the methylation markers mentioned in Table 4 can furthermore also be analyzed, which further allows conclusions to be drawn about the stage of the tumor.

Thus, the invention provides furthermore a method for diagnosing lung cancer, in which the methylation of a set of methylation markers, e.g., in cfDNA from a liquid biopsy sample of a patient, is determined, wherein the set of methylation markers comprises the following 10 positions (see also Table 2):

| ID | Chromosome | Position |
|---|---|---|
| 596 | chr11 | 57006229 |
| 1717 | chr15 | 28262724 |
| 2636 | chr18 | 61144199 |
| 2805 | chr19 | 46823441 |
| 4674 | chr2 | 176964685 |
| 4999 | chr2 | 225642035 |
| 5071 | chr3 | 14960020 |
| 5576 | chr4 | 13525705 |
| 6105 | chr5 | 140475760 |
| 6434 | chr6 | 46386723. |

It has been demonstrated that said markers are particularly informative if the kNN algorithm is used for analysis. Using said markers, especially the presence of a tumor can be analyzed.

Alternatively or additionally, the set of methylation markers can comprise the following 10 positions (see also Table 3):

| ID | Chromosome | Position |
|---|---|---|
| 650 | chr11 | 64993331 |
| 2995 | chr1 | 17568007 |
| 4233 | chr2 | 50574690 |
| 4241 | chr2 | 50574708 |
| 4428 | chr2 | 111874494 |
| 4447 | chr2 | 121276804 |
| 5537 | chr4 | 1666074 |
| 5538 | chr4 | 1666075 |
| 6524 | chr6 | 83604790 |
| 7164 | chr7 | 69971740. |

It has been demonstrated that said markers are particularly informative if the RT algorithm is used for analysis. Using said markers, especially the entity of a tumor can be identified.

Optionally, especially if, furthermore, the stage of a tumor is to be identified (e.g., a distinction is to be made between an early (I+II) and a late (III+IV) stage of a lung carcinoma), the set of methylation markers can furthermore comprise all the positions listed in Table 4. In this case, the SVM algorithm can be used for analysis.

In the case of regions which could not be validated using samples from early lung carcinoma stages, could be signatures specific for metastases, for example. Therefore, said regions were used for calculation of the staging parameter, i.e., for calculation of the stage. So far, the staging parameter described in this work can distinguish the late stages of lung carcinoma from early stages with 80% accuracy. In general, the staging parameter should only be used as an indication. If the developed panel detects a lung carcinoma, it would be additionally advisable to generate therapeutically relevant information, e.g., with regard to the size or location of the tumor, by imaging methods, such as, e.g., MRI, CT or PET CT. It is thus also not essential to coanalyze the stage-based methylation markers in each case.

In the context of the invention, the lung cancer can be NSCLC or SCLC, preferably NSCLC. The NSCLC is preferably an adenocarcinoma or squamous cell carcinoma. It has been demonstrated that markers according to the invention can differentiate between these entities and are therefore suitable for differential diagnosis.

The diagnosis according to the invention makes it possible to state the presence of a tumor, the entity of a tumor (especially the differentiation between adenocarcinoma and squamous cell carcinoma), the tumor stage and/or the prognosis. Most important is the statement about the presence and entity of the tumor. Further statements can optionally also be made by means of supplementary methods, if the presence of a tumor has been established according to the invention. However, the method according to the invention optionally also allows already a statement about the presence of a tumor, the entity of a tumor (especially the differentiation between adenocarcinoma and squamous cell carcinoma) and the tumor stage and preferably the prognosis. The term of diagnosis thus includes differential diagnosis.

In contrast to hitherto known methods, the method according to the invention is also suitable for early detection of lung cancer, i.e., also for diagnosis in stage I or II. Advantageously, said diagnosis is furthermore also possible on the basis of a liquid biopsy sample, i.e., for example a blood sample, so that other tissue does not necessarily have to be removed from the patient.. According to the invention, e.g., a liquid biopsy sample of a patient is therefore analyzed.

In addition, the method according to the invention can advantageously also be reliably carried out on the basis of lung biopsy tissue. In this case, it is also possible to carry out a “paired biopsy” and to therefore examine and compare in parallel tissue from lung biopsies of the presumably diseased lung and the presumably healthy lung of a patient. In the clinic, usually only the tumor or suspicious tissue is biopsied, with previously collected data sets of healthy tissues serving as a reference if necessary.

Preferably, the patient is a human being. In general, the word patient is used synonymously with subject. It may be a patient with symptoms suggesting that the patient has a lung tumor. However, it may also be a subject without symptoms. The subject or patient can be a patient at risk of a lung tumor. These include subjects who, because of certain risk factors and/or their lifestyle (e.g., smoking, use of e-cigarettes or other increased exposure to carcinogenic agents, symptoms), have an increased risk of a lung cancer disease and/or exhibit radiological abnormalities. The patient may also be a patient with a previously treated lung tumor, such as one who has undergone surgery, in which case tumor recurrence and/or metastasis may be investigated.

In general, the cfDNA can be extracted from a plurality of body fluids. For example, successful extraction from blood plasma and serum, pleural effusion or urine has already been described in the literature. According to the invention, the liquid biopsy sample can be blood, plasma, serum, sputum, bronchial fluid and pleural effusion. Preferably, it is derived from blood, e.g., serum or plasma, preferably plasma. Since pleural effusion only occurs in the course of the disease, this material is especially suitable for the detection of later stages. cfDNA extraction from plasma or serum is distinctly more rapid and cost-effective than from urine, which makes these materials more interesting for screening. Lastly, cfDNA stability is relevant, since cfDNA is more stable in plasma than in serum.

In one embodiment, the invention provides means which are suitable for diagnosing lung cancer using a method according to the invention by examination of the methylation of a set of methylation markers, e.g., in cfDNA from a liquid biopsy sample of a patient. The means are preferably also suitable for diagnosing lung cancer using a method according to the invention by examination of the methylation of a set of methylation markers in a different sample of a patient, especially a solid tissue sample from a tumor or a tissue in which a tumor is suspected or from a lung biopsy.

In this context, the means comprises oligonucleotides which can hybridize to DNA (e.g., cfDNA or DNA derived therefrom, e.g., by bisulfite conversion) which comprises or consists of methylation markers according to the invention. Methylation markers from the subgroups mentioned in the claims are preferred in this context. “Can hybridize” is to be understood to mean a specific hybridization, especially under stringent conditions, as outlined in the experimental section for instance.

Suitable oligonucleotides are, e.g., oligonucleotides which can hybridize to the regions mentioned in Table 1a, 1b and/or 1c, preferably in Table 1a, because they are complementary to these regions or a fragment thereof which comprises at least 20 nucleotides, e.g., when coupling to a solid support, preferably 60-352, optionally 100-190 or 135-157 nucleotides. For this, the length depends, inter alia, on the base composition or sequence and the hybridization temperature and on the technique selected. Since the DNA is double-stranded, the oligonucleotides can be complementary to the strand in the 5′-3′ direction or to the strand in the 3′-5′ direction, or to both. What is important is that the selected oligonucleotides cannot hybridize to regions other than those mentioned in the tables, which is likewise a prerequisite for a specific hybridization. Exemplary suitable oligonucleotides which can hybridize to the regions on Chromosome 1 mentioned in Tables 1a, 1b and 1c are listed in Table 5. A person skilled in the art is capable of selecting oligonucleotides suitable for other markers on the basis of the information disclosed herein about the markers.

Such oligonucleotides can optionally comprise further components, e.g., spacers or linker regions.

The oligonucleotides according to the invention can, e.g., be coupled to a solid support or are oligonucleotides which have been coupled to a solid support. Such coupling is, e.g., possible by means of adapters or tags. One option for this is coupling to biotin, which can bind (or has already bound) to streptavidin or avidin, which is coupled to the solid support.

The solid support can, e.g., be a gene chip, a globule or bead, e.g., a magnetic bead, or a column matrix. The support thus allows simple separation of the hybridized DNA. In the Example section, magnetic beads are described, which have been coupled via streptavidin-biotin binding to oligonucleotides which specifically hybridize to the regions mentioned in Table 1 and can be used as capture probes. Optionally, the means according to the invention comprise 638 oligonucleotides, e.g., capture probes, which can hybridize to all the methylation markers mentioned in Table 1.

Alternatively or additionally, the oligonucleotides according to the invention may also be a kit comprising PCR primers for amplification of regions which comprise the methylation markers or (especially in the case of regions from Table 1) consist thereof. PCR primers preferably have a length of approx. 12-40, optionally 15-25 nucleotides, which can hybridize to said regions. Such a kit can also comprise blocking oligonucleotides or detection probes, which, after bisulfite conversion, can specifically bind to previously methylated DNA or unmethylated DNA. Such oligonucleotides can, e.g., be used in PCR-based methods according to the invention.

An analysis by PCR is especially appropriate if only a limited number of markers is to be analyzed, i.e., for example the markers in the abovementioned genes. Preferably, this method analyzes the markers defined in Table 2, alternatively or additionally also the markers defined in Table 3, so that appropriate oligonucleotides can be selected accordingly.

Optionally, one or more primers suitable for multiplex PCR can be selected. Probes for detection are preferably labeled with suitable dyes.

The invention also provides a method in which the means according to the invention are used for diagnosis of lung cancer in a sample of a patient, wherein optionally cfDNA from a liquid biopsy sample of a patient (also referred to as subject) is examined. Owing to the selection of markers, other samples, e.g., from biopsies and bronchoscopies or from tissue samples collected during surgery, can, however, also be examined using the means according to the invention, especially using those which comprise markers from Table 1 a, b and/or c, preferably all the markers from Tables 1a and 1b and optionally also from Table 1c. Biopsies can also be collected from the outside if necessary under imaging.

If sequencing data are to be used, the bioinformatic evaluation pipeline poses a further problem. Conventional gDNA-WGBS libraries are usually aligned using the “Bismark” algorithm after processing. The results of the alignment can then subsequently be analyzed by numerous evaluation pipelines, with genome-wide DNA methylation signatures being extracted. The WGBS experiment of the circulating-DNA carried out in the exemplary embodiments was the first of its kind. It was found that the cfDNA libraries have a different complexity as well as fragment distribution compared to conventional gDNA libraries (see section 1.1.2.5). This might be the reason why the “Bismark” algorithm most commonly used in the prior art provided an unsatisfactory mapping efficiency of only 70%. It is for this reason that further algorithms were tested. The best results, with a mapping efficiency of at least 98%, were provided here by the “Segemehl” algorithm (see section 1.1.2.5).

Therefore, in the embodiment of the invention that is based on sequencing of bisulfite-converted cfDNA, the Segemehl algorithm is particularly used to align (i.e., to arrange) the sequencing information of the cfDNA with respect to a reference genome. The Segemehl algorithm is found under https://www.bioinf.uni-leipzig.de/Software/segemehl/ and is described in more detail in, e.g., Otto et al. (Otto et al. [2012] Bioinformatics 28: 1698-1704). Version 0.2.0 can be used, as in the example described below, but also another version, such as 0.3.4..

Another aspect of the invention provides a method according to the invention for diagnosing a lung tumor, comprising the following steps:
a. extracting cfDNA from a liquid biopsy sample or genomic DNA from a lung biopsy tissue sample or a solid tissue sample, which is collected, e.g., during surgery, optionally cfDNA from a liquid biopsy sample,
b. carrying out a bisulfite conversion,
c. producing a whole-genome bisulfite sequencing library,
d. enriching the DNA regions comprising the defined methylation markers, wherein these are preferably contacted with a means according to the invention for diagnosis,
e. sequencing the enriched DNA regions,
f. aligning the sequencing data against a reference genome using the Segemehl algorithm,
g. calculating the methylation rates.

Means and methods for extracting genomic DNA, for extracting cfDNA from plasma, quantification, quality control (QC) and bisulfite conversion are known to a person skilled in the art from the state of the art and/or described herein.

The converted DNA, e.g., cfDNA, can be used for the production of the libraries. Library preparation is done in two steps. In the first step, e.g. as described in section 1.1.2.4, a WGBS Library is produced from each sample, which contains information about the entire methylome or the zfDNA methylome of the corresponding patient. However, as only the specific, differentially methylated regions are sequenced and analyzed in the further course, these can be enriched from the entire methylome. This can be done as the second step on the basis of the Whole Genome Bisulfite Sequencing Library.

Various sets of methylation markers according to the invention can be used for enrichment, e.g., the markers identified in cfDNA for the first time in the context of the present work from Table 1a, all markers from Table 1a, alternatively or additionally the markers from Table 1b and/or 1c. It is, however, also possible to use only methylation markers for which particular significance has been found in the context of the classification, especially for the presence of a tumor (Table 2) or for the determination of the entity of the tumor (Table 3), but optionally also for the determination of the tumor stage (Table 4).

For enrichment, e.g., capture probes can be used. Said capture probes can cover the entire plasma panel or parts thereof (see section 1.2.1).

The enriched library can be subjected to a QC as well as quantified (see section 1.1.2.2). It is preferably sequenced, e.g., on the “MiSeq” (“Illumina”, USA) (see section 1.2.2). The sequencing data can, e.g., be stored in “FastQ” format and subsequently be analyzed (see, for example, section 1.2.3). Preferably, not the entire methylome is to be analyzed, but only defined methylation markers. Preferred methylation markers are, e.g., the 638 regions defined in Table 1 (plasma panel).

As mentioned, for the analysis, especially the Segemehl algorithm is used for alignment against a reference genome. Thereafter, the methylation patterns are calculated.

The format of the “Segemehl” output file is one that is different from the typical “Bismark” format. Therefore, a suitable “Segemehl″-compatible analysis pipeline may be used. In this context, e.g., the “Bisulfite Analysis Toolkit” can be mentioned by way of example. This software of modular construction can be used on numerous computing clusters and expanded by further software as well as own scripts. For the identification of the differentially methylated markers suitable for diagnosis of lung cancer, the analysis pipeline can be supplemented with own bioinformatic scripts, e.g., the ones disclosed herein.

As an alternative to the diagnostic method via sequencing, it is also possible, on the basis of the results according to the invention, to carry out an analysis via PCR. This is especially relevant to smaller subgroups of the defined markers, e.g., if initially a sample of a patient is to be examined only for the presence of a tumor and/or the determination of the tumor entity. In this case, e.g., suitable primers can be used to amplify regions of the e.g., cfDNA and to detect the positions mentioned in Table 2 and/or 3. This can be done from purified, bisulfite-converted DNA, e.g., by real time PCR. Multiplex PCRs or parallel mixes can, however, also be used.

As internal control, e.g., beta-actin can be analyzed to check whether the amount of total DNA in the sample is sufficient. For this, e.g., cfDNA from a liquid biopsy, preferably from plasma, can be purified, bisulfite-converted and again purified, as described, e.g., in the exemplary embodiments. Blockers and detection probes can further be used for PCR that specifically recognize the bisulfite-converted unmethylated sequences within the regions and block their amplification so that the methylated sequences are preferentially amplified. Methylation-specific probes then exclusively detect methylated sequences which were amplified during the PCR.

Comparable methods are already described, e.g., for the Epi proLung Kit (Epigenomics AG, Berlin), and can be adapted for the methylation markers relevant according to the invention, e.g., from Tables 2 and 3. Evidently, it is also possible to additionally examine further methylation markers, e.g., from the plasma panel, with this method, e.g., more than 25 differentially methylated positions or more than 30 differentially methylated positions, preferably comprising the methylation markers mentioned in Tables 2 and 3 and/or lying within the regions mentioned in Table 1, preferably both.

The methylation patterns established in the sample of a patient (via sequencing-based methods or PCR-based methods), i.e., the results of the methylation marker analysis, can be correlated with the patterns known herein for tumors, optionally a certain entity and/or a certain stage, as specified, e.g., in the tables. According to the invention, this allows conclusions to be drawn about the presence, entity, stage and/or prognosis of a lung tumor, thus permitting a reliable advanced diagnosis.

According to the invention, this diagnosis can be used for selecting a therapy or for deciding on the commencement of a therapy in the event of a tumor being present.

In one embodiment, the invention thus also relates to a method for treating a lung tumor, comprising a diagnostic method according to the invention, wherein, in the event of a tumor being present, said tumor is treated. Advantageously, the entity of the tumor can also be established, allowing the selection of a therapy suitable for, e.g., an adenocarcinoma or a squamous cell carcinoma. A suitable therapy can, e.g., comprise the administration of suitable medicaments or combinations of medicaments and/or irradiation.

Alternatively, the diagnostic method can be used to carry out further diagnostic steps, such as the collection of a solid biopsy and or imaging methods, in the event of a tumor being detected.

Another aspect of the invention provides for the use of a method according to the invention or of a means according to the invention for diagnosing lung cancer, wherein the diagnosis allows a statement about the presence of a tumor, about the entity of a tumor, about the tumor stage and/or about the prognosis, preferably about the presence and entity of the tumor, optionally about all at the same time.

In summary, it can be stated that, in the context of the present invention, it was possible for the first time to develop an NGS panel which is based on, inter alia, genome-wide cfDNA methylation signatures from plasma. Said plasma panel could be successfully validated using liquid biopsies of a patient cohort (n=12). However, the method according to the invention is explicitly distinguished by the fact that, due to the selection of markers, it is also particularly well suited for an examination of, e.g., tissue samples taken during surgery or lung biopsy tissue, in addition to the examination of zfDNA from a liquid biopsy. During the pilot study, the plasma panel distinguished malignant lung tumors with 100% accuracy as early as from stage I, identified the most common NSCLC subtypes and provided further information with regard to determining the stage of the lung tumors (staging).

The invention will be elucidated below by means of examples which are intended to illustrate, but not to limit, the invention. All the references cited in this application are fully incorporated herein by reference in their entirety.

### LEGEND

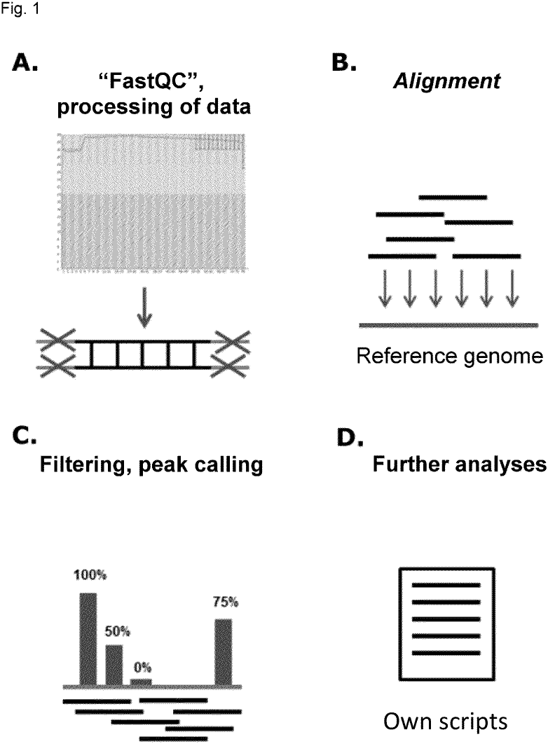

FIG. 1: The analysis of the WGBS sequencing data was performed in several steps. A. First, the data were subjected to a QC (e.g., with FastQC) and subsequently processed. B. Then, the processed data were aligned against a reference genome (e.g., “HG19”) and subsequently C. used to calculate the DNA methylation rates. The positions at which a methylation rate was ascertained were then filtered according to certain criteria (e.g., coverage and CpG context) and lastly D. subjected to further analyses using own scripts.

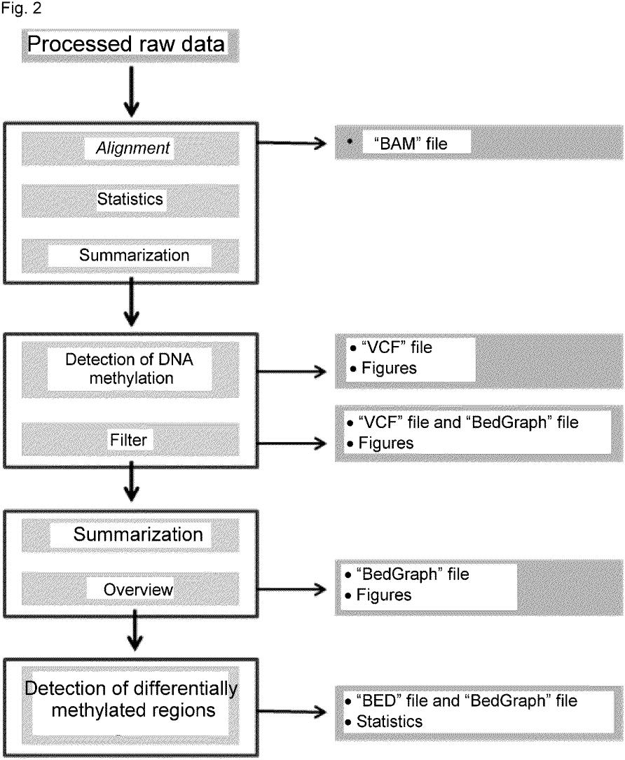

FIG. 2: Processed sequencing data were aligned against the “HG19” reference genome, use being made of the “Bisulfite Analysis Toolkit” with use of the Segemehl algorithm. Furthermore, the detection of DNA methylation rates and differentially methylated regions as well as the generation of overview charts were performed.

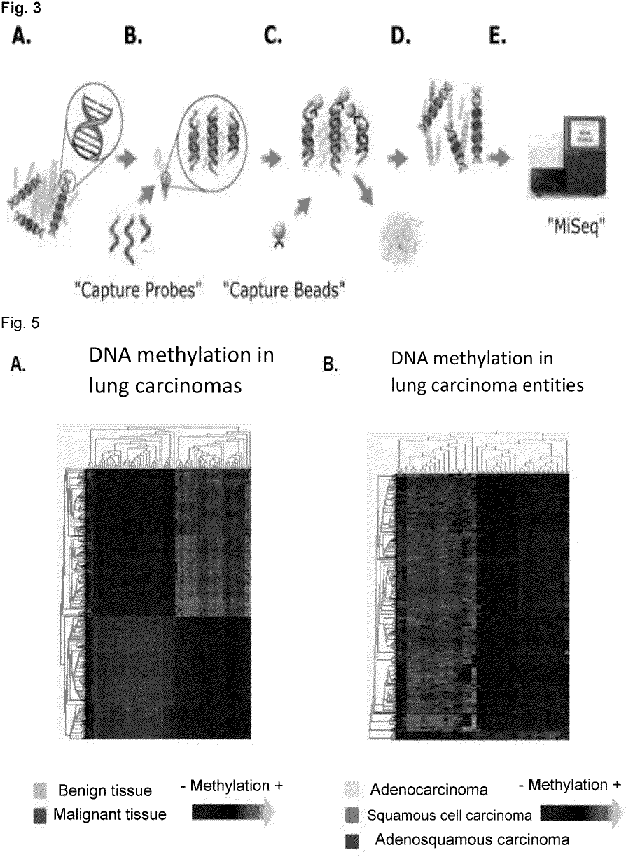

FIG. 3: The enrichment of differentially methylated regions of the set of methylation markers important according to the invention was divided into multiple steps. A. First, as described in, e.g., section 1.1.2.4, WGBS libraries were produced,. For validation, they can be pooled equimolarly; if this is being carried out for diagnosis of patients, which depends on the sequencer and its capacity and on the sample volume, then individual samples can be individually labeled by “barcoding” and sequenced together to separate the samples again bioinformatically. B. The 638 differentially methylated regions were then hybridized to “Capture Probes”, in this case using the “SeqCap Epi Enrichment Kit”, C. enriched using “Capture Beads” and lastly D. amplified in a PCR reaction. E. The completed NGS libraries were then quantified, subjected to a QC and sequenced on the “MiSeq”.

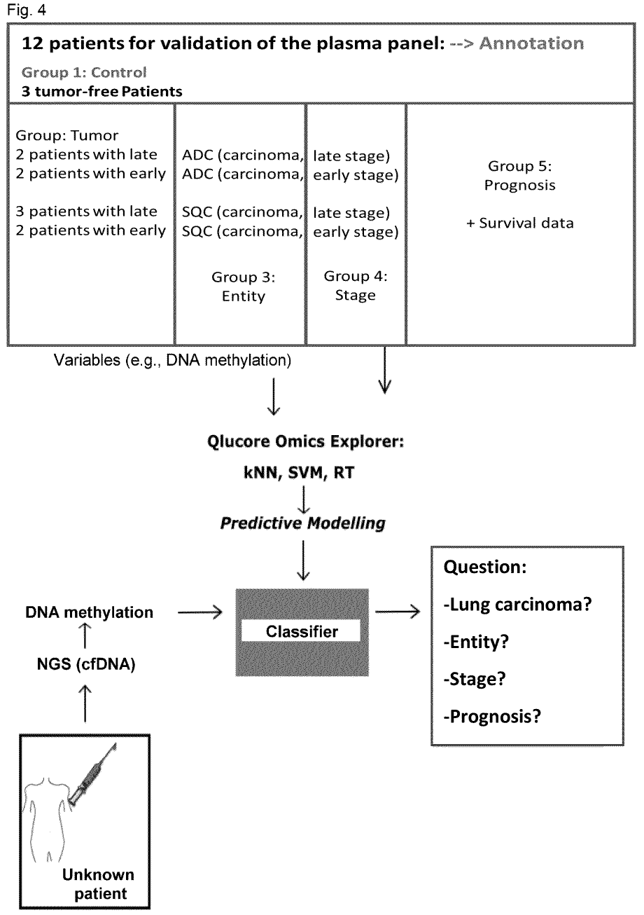

FIG. 4: The functional principle of a classifier. From the data of the validation cohort (12 patients), an annotation file is first generated, which is additionally loaded into “Qlucore Omics Explorer” software with the ascertained DNA methylation rates of the regions present in the plasma panel (see Table 1). The DNA methylation data (variables) and the annotation file are used by implemented algorithms (“k-Nearest Neighbors Algorithm” (kNN), “Support Vector Machines” (SVM) and “Random Trees” (RT)) to create an optimal model. This process is referred to as predictive modeling. After the optimal classifier has been generated, it is capable of analyzing the cfDNA methylation pattern of an unknown patient and thus of making a diagnosis (adenocarcinoma (ADC), squamous cell carcinoma (SQC)).

FIG. 5: Results of the differential methylation analysis with HM 450K. The hierarchical cluster analysis of 40 surgical preparations and the corresponding controls thereof identified A. 898 differentially methylated CpG loci in tumor samples (q< 1 × 10⁻²³, σ/σₘₐₓ> 0.4) (left half: three tumor samples on the far left and then benign tissue; right half: tumor tissue) and B. 1167 differentially methylated CpG loci in different lung carcinoma entities (FDR < 1 × 10⁻⁴) (light upper edge: adenocarcinoma; gray upper edge: squamous cell carcinoma; dark upper edge: adenosquamous carcinoma. Results: dark: less methylation; light: much methylation).

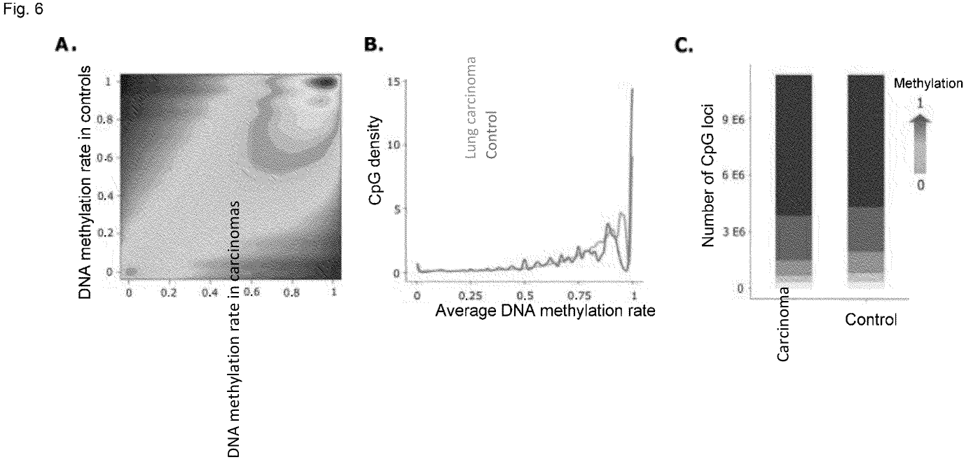

FIG. 6: The DNA methylation rates ascertained using the “BAT_calling” and “BAT_filter_vcf” modules were loaded into the “BAT_summarize” module of the “Bisulfite Analysis Toolkit”. A. The scatter plot clearly shows that the lung carcinoma group can be distinguished from the control group (tumor-free patient cohort) on the basis of the DNA methylation pattern. B. The average and C. the staggered plots of the DNA methylation rates per group illustrate the genome-wide hypermethylation of the lung carcinoma group in comparison with the control group.

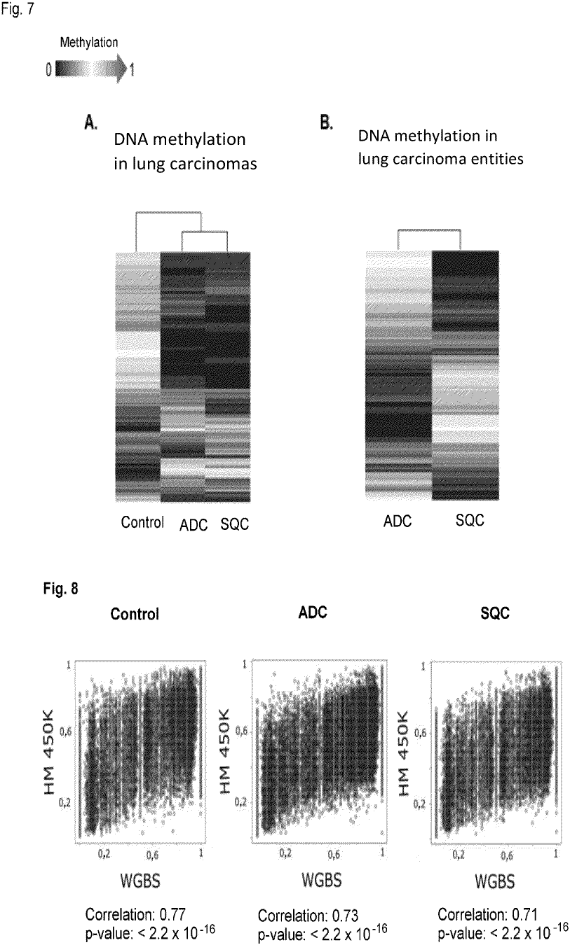

FIG. 7: The ascertained cfDNA methylation patterns were normalized and subjected to a hierarchical cluster analysis. In this case, of the differentially methylated CpG loci identified, A. 18 000 were specific for lung cancer and B. 44 000 were specific for the particular entity (adenocarcinoma (ADC), squamous cell carcinoma (SQC)).

FIG. 8: “Pearson” correlation analysis of the DNA methylation values detected using the two methods (HM 450K and WGBS) (adenocarcinoma (ADC), squamous cell carcinoma (SQC)).

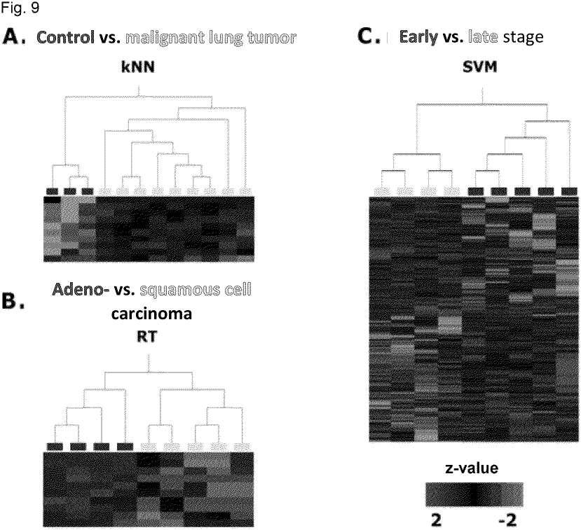

FIG. 9: The ascertained cfDNA methylation rates were loaded into “Qlucore Omics Explorer” software and analyzed using the following classification algorithms: “k-Nearest Neighbors Algorithm” (kNN), “Support Vector Machines” (SVM) and “Random Trees” (RT). A high z-value means a strong methylation. A. The kNN algorithm was able to distinguish healthy patients (control) from those suffering from a malignant lung carcinoma by analyzing 10 differentially methylated positions (markers). Both the early (I, II) and the late (III, IV) stages of lung carcinoma were classified with 100% accuracy (light bars on the top side of the figure: malignant lung tumor; dark bars (3 columns on the left): control). In the case of 9 of the 10 positions, there is a stronger methylation in the tumor tissue, in the case of one, a weaker methylation. B. The RT algorithm analyzed 10 positions to ascertain the entity of the tumor with 100% accuracy (light bars on the top side of the figure (6 columns on the right): squamous cell carcinoma; dark bars (4 columns on the left): adenocarcinoma). For all the markers shown, there is a stronger methylation in the case of adenocarcinoma than in the case of squamous cell carcinoma. C. The late tumor stages (III, IV) could be identified with 80% accuracy using the SVM algorithm; for this 523 positions were analyzed (light bars on the top side of the figure (4 columns on the left): early stage (I, II); dark bars on the top side of the figure (5 columns on the right): late stage (III, IV)). Thereby, the evaluated positions are partly in the early, partly in the late stages more methylated.

### EXAMPLES

### 1.1 Methods: Development of the Plasma Panel

To enable noninvasive lung cancer diagnostics, in the context of the invention, a suitable panel, i.e., a set of methylation markers, was developed for DNA methylation analysis in blood plasma. The set of methylation markers is therefore also referred to as the plasma panel. The development of the plasma panel was carried out in three independent approaches. In the first approach, it was checked whether DNA methylation is generally suitable as biomarker for lung cancer diagnostics (see section 1.1.1). For this purpose, 40 lung carcinomas and the corresponding controls thereof were analyzed using the “Illumina Infinium Human Methylation450K BeadChip” (HM 450K). The method identified distinct, tumor-specific DNA methylation signatures. Next, as described in section 1.1.1, the regions having the strongest differences in DNA methylation were ascertained and incorporated into the panel.

In the second approach, it was examined whether tumor-specific DNA methylation signatures can also be detected in the blood plasma of the patients affected (see section 1.1.2). For this, circulating cell-free DNA was extracted from the plasma of adenocarcinoma (n=5) and squamous cell carcinoma patients (n=4) and subsequently combined into 3 pools. Plasma of a tumor-free patient cohort (n=19) served as control. Detailed information about the patients is compiled in section 1.1.2. As a result of pooling, individual DNA methylation patterns were largely eliminated, and the general tumor- or lung-specific signatures were, by contrast, emphasized. Then, the cfDNA pools were subjected to whole-genome bisulfite sequencing (WGBS; see section 1.1.2.4). The method detected several thousand aberrantly methylated CpG loci which were not only tumor-specific, but also entity-specific. Of these, the most suitable regions were selected for differentiation for the plasma panel (see section 1.1.2.5.5). Since diagnosis according to the invention is preferably to be performed on the basis of liquid biopsies, the methylation markers identified here are of particular significance.

In the third approach, the plasma panel was supplemented by 59 tumor-specific and prognostically relevant CpG loci from further studies (see section 1.1.3).

### 1.1.1 Detection of Aberrant DNA Methylation in Primary Tumor Tissue

The HM 450K data set contained information about the methylation status of 40 lung carcinomas (adenocarcinomas and squamous cell carcinomas) and their corresponding controls. The data set was evaluated using the “Qlucore Omics Explorer” software (version 3.2, “Qlucore”, Sweden) and yielded:
1.) 897 CpG loci (t-test: FDR < 1 × 10⁻²³, σ/σₘₐₓ > 0.4) which were differentially methylated between the tumor tissue and healthy lung tissue.
2.) 1167 CpG loci (t-test: FDR < 1 × 10⁻⁴) which differentiated between the adenocarcinoma tissue and squamous cell carcinoma tissue.

To ascertain the CpG loci having the strongest differences in DNA methylation, the two lists were first filtered according to differential methylation greater than 35% (avg. beta > 0.35) and annotated against the “HG19” reference genome using “Bedtools” (version 2.2.6, “The University of Utah”, USA). All CpG loci which were located within common SNPs (≥1% of the population) and were non-protein-coding were discarded. The remaining loci were incorporated into the final plasma panel (Table 1).

### 1.1.2 Detection of Aberrant DNA Methylation in Blood Plasma

According to the invention, circulating cell-free DNA is used for noninvasive diagnostics of solid tumors. If a patient is suffering from a malignant tumor disease, the total amount of circulating DNA also contains the tumor DNA, which contains all therapeutically and prognostically relevant information about the genetic and epigenetic characteristics of the tumor. Therefore, cfDNA must be isolated from blood or blood plasma. Since cfDNA can be extracted from blood plasma only in a very low amounts, a method was chosen for this purpose that very specifically and efficiently enriches zfDNA without isolating further components of plasma.

For this, e.g., the “PME free-circulating DNA Extraction Kit” (“Analytik Jena”, Germany; see section 1.1.2.1) can be used. It contains a polymer which only complexes short-stranded dsDNA fragments highly specifically. The polymer-cfDNA complex is subsequently precipitated and purified. After purification, the complex compound can be disassociated. The released DNA is purified from the polymer and concentrated in further steps, e.g. by binding to a silica column. Other methods based, e.g., on the same or similar principles of action can be used, too. The resultant product is very clean and can also be used for sensitive NGS-based analysis methods such as, e.g., WGBS.

### 1.1.2.1 Extraction of Circulating, Cell-Free DNA (cfDNA) From Blood Plasma

Blood plasma was prepared and shipped on dry ice. For this purpose, whole blood was centrifuged within 30 min of collection at 1500 g for 10 min. After centrifugation, the plasma supernatant was carefully pipetted off, aliquoted into “CryoPure” tubes (“Sarstedt AG&Co”, Germany) and immediately frozen at -80° C.

The frozen plasma samples were slowly thawed under lukewarm water and subsequently centrifuged at 4500 g for 10 min. The pellet was discarded, and the clear supernatant was transferred into a 10 mL tube and processed using the “PME free-circulating DNA Extraction Kit” according to the manufacturer’s instructions.

### 1.1.2.2 Quantification and Quality Control (QC) of Extracted cfDNA

The cfDNA was quantified fluorometrically using the “Qubit dsDNA High Sensitivity Assay Kit” (“Thermo Fisher Scientific”, USA). For this purpose, 1 µL of each sample was mixed with 198 µL of “Qubit dsDNA HS Buffer” and 1 µL of “Qubit dsDNA HS Reagent”, incubated for 2 min and subsequently measured in the “Qubit 2.0” fluorometer (“Thermo Fisher Scientific”, USA). The “Qubit dsDNA HS Reagent” was a dye which generates a very weak fluorescent signal under normal conditions. However, in the presence of double-stranded DNA (dsDNA), it intercalates into the dsDNA, alters its structure and generates a strong fluorescent signal. Neither single-stranded DNA (ssDNA) nor RNA is bound. Therefore, the signal intensity exclusively correlates with the amount of dsDNA present in the sample.

The quality of the extracted cfDNA was analyzed with the aid of the “Agilent 2100 High Sensitivity DNA Kit” (“Agilent”, USA). The method was capillary gel electrophoresis. First, the “Gel-Dye Mix” had to be prepared. For this 300 µL of the gel matrix were added to 15 µL of the dye concentrate, mixed and transferred to a “Spin Filter”. Centrifugation was carried out at 2240 g for 10 min. Next, the DNA chip was placed and equilibrated in the “Priming Station”. Regarding this, 9 µL of the “Gel-Dye Mix” were pipetted into the well intended for the equilibration process. The plunger of the “Priming Station” was adjusted to one milliliter. After the “Priming Station” was firmly closed, the plunger was depressed for one minute. Lastly, the remaining wells of the chip were loaded according to the manufacturer’s instructions. The chip was incubated for 1 min and directly measured afterwards. During the incubation time, a fluorescent dye present in the “Gel-Dye Mix” intercalated between the bases of the dsDNA. The dsDNA fragments were subsequently drawn through the microscopically small capillaries of the “Agilent 2100 Bionalyzer” (“Agilent”, USA) and, in the course of this, resolved and detected according to fragment size.

### 1.1.2.3 Bisulfite Conversion of cfDNA

For whole-genome analysis of the DNA methylation pattern, e.g., by the HM 450K or WGBS, DNA is subjected to PCR-based whole-genome amplification. DNA polymerases cannot distinguish between cytosines and 5-methylcytosines, so that, during the reaction, all 5-methylcytosines are replaced with cytosines. The newly synthesized strands are not remethylated.

In order to be able to distinguish cytosines from 5-methylcytosines, the sample is subjected to a treatment with sodium bisulfite prior to PCR. This process is referred to as bisulfite conversion, which involves conversion of all unmethylated cytosines into uracils. By contrast, the methylated cytosines remain unaltered under the chosen reaction conditions. The reaction of bisulfite conversion is described in NEB, N.E.B. Bisulfite Conversion (available under: http://www.neb-online.de/wp-content/uploads/2015/04/NEB_epigenetik_bisulfit3.jpg), and in Clark et al. (Clark et al. [1994] Nucl. Acids Res 22: 2990-2997).

The bisulfite conversion of cfDNA can, e.g., be carried out using the “EZ DNA Methylation-Gold™ Kit” (“Zymo Research”, USA). For this, 10 ng of the previously extracted cfDNA were dissolved in 20 µL of water, admixed with 130 µL of “CT” conversion reagent and processed in the thermal cycler under the following program: 10 min at 98° C., 2.5 h at 64° C., up to 20 h at 4° C. In the next step, the bisulfite-converted samples were desulfonated and purified. For this purpose, they were admixed with 600 µL of “M-Binding Buffer”, pipetted onto the “Zymo-Spin™ IC” columns and centrifuged at 10 000 g for 30 s. Then, 100 µL of “M-Wash Buffer” were added to the columns. The columns were centrifuged at 10 000 g for 30 s and treated with 200 µl of “M-Desulphonation Buffer” for 20 min. After subsequent centrifugation at 10 000 g for 30 s, the “Zymo-SpinTM IC” columns were washed with 200 µL of “M-Wash Buffer” and centrifuged at 10 000 g for 30 s to remove remaining liquids, and the DNA was eluted at 10 000 g for 30 s with 15 µL of “Elution Buffer”.

### 1.1.2.4 Whole-Genome Bisulfite Sequencing (WGBS)

In order to be able to analyze the cfDNA methylation profile at the genome-wide level, the previously bisulfite-converted samples were subjected to WGBS. WGBS is an NGS-based method (next-generation sequencing). Nowadays, there are numerous technologies which make NGS possible. The NGS technology which is the most common and is also used here is offered by “Illumina” (USA). The underlying sequencing reaction is fluorescence-based and is done on a glass support, also called flowcell. To immobilize the DNA fragments on the flowcell, specific “Illumina” adapters (short oligonucleotides) are first ligated. The sample is then subjected to a denaturation reaction. Since not only the adapter binding sites but also primers are present on the flowcell, the ssDNA fragment to be sequenced “folds over”. During the subsequent PCR reaction, the DNA strands are amplified. This process is referred to as bridge amplification. It yields, through the progressive amplification at delimited positions, so-called sequencing clusters, which subsequently dissociate. Cluster formation is followed by the actual sequencing reaction, during which there is incorporation of DNA bases which generate fluorescent signals of different wavelengths depending on the base incorporated. After every completed incorporation cycle, said fluorescent signals are detected and thus provide the information about the base sequence within a read.

Different “Illumina” platforms can be used depending on the desired throughput. For the sequencing of specific regions, so-called panels, such as the panel or set of methylation markers identified according to the invention, the relatively rapid and relatively cost-effective “MiSeq” platform is generally sufficient. However, sequencing can, e.g., also be carried out on the “NextSeq 500” or “HiSeq” sequencing platforms or other suitable sequencing platforms.

### 1.1.2.4.1 Creation of WGBS Libraries

During bisulfite conversion, DNA is highly stressed by the reagents used and thus degraded to a high degree. This is why conventional WGBS protocols use very high amounts of DNA, at least 500 ng. Since cell-free, circulating DNA is, on the one hand, already very highly fragmented from the beginning and can, on the other hand, only be obtained in a very low amount, the production of WGBS libraries using conventional kits is difficult at present.

Therefore, the “Accel-NGS® Methyl-Seq DNA Library Kit” (“Swift Biosciences”, USA) was established for the following experiments. The kit was specifically developed for WGBS of cfDNA. Even with zfDNA amounts of less than 10 ng, complex WGBS libraries can be generated. The central role is played by the enzyme “Adaptase”, which adds a 10 nt long overhang at the 3′ end of the bisulfite-converted ssDNA. Said overhang allows better ligation of the sequencing adapters and thus more efficient library production. Therefore, according to the invention, a method for the preparation of the WBGS libraries is preferably used, which inserts a 10 nt long overhand at the 3′ end of the bisulfit converted ssDNA by means of the enzyme adaptase.

Library production was carried out in four steps using the “Accel-NGS® Methyl-Seq DNA Library Kit” (“Swift Biosciences”, USA): treatment with the enzyme “Adaptase”, extension, ligation, PCR. For the treatment with the enzyme “Adaptase”, 10 ng of bisulfite-converted cfDNA were taken up in 15 µL of water and denatured at 95° C. for 2 min. Then, 25 µL of the “Adaptase Reaction Mix” were added to the sample, carefully mixed and processed in the thermal cycler (program 1: 37° C. for 15 min; 95° C. for 2 min; 4° C.; for all programs, the lid of the thermal cycler was preheated). Next, extension was carried out. For this purpose, the sample was admixed with 44 µL of “Extension Reaction Mix”, carefully mixed and incubated in the thermal cycler (program 2: 98° C. for 1 min; 62° C. for 2 min; 65° C. for 5 min; 4° C.).

The product was purified. For this, e.g., “SPRI Beads” (“Beckman Coulter”, USA) can be used. This was followed by ligation, for which 15 µL of the product were admixed with 15 µL of “Ligation I Reaction Mix” and processed in the thermal cycler (program 3: 25° C. for 1 min; 4° C.). Also in this step, the finished product was purified using “SPRI Beads” (“Beckman Coulter”, USA). Lastly, PCR was carried out. For this, 5 µL of the respective index and 25 µL of the “Indexing PCR Reaction Mix” were added per sample. The finished PCR reaction was incubated in the thermal cycler (program 4: 98° C. for 30 s; PCR cycles: 98° C. for 10 s; 60° C. for 30 s; 68° C. for 1 min (7-9 cycles); 4° C.) and purified by means of the “SPRI Beads” (“Beckman Coulter”, USA) according to the manufacturer’s instructions.

The finished WGBS libraries were quantified and tested for their quality as described in section 1.1.2.2.

### Purification of “SPRI Beads”

The samples were transferred into 1.5 mL Eppendorf reaction tubes and admixed with “SPRI Beads” (“Beckman Coulter”, USA) in the prescribed ratio (Tab. A). Then, the samples were mixed and incubated at room temperature for 5 min. Since the beads were magnetic, the principle of magnetic separation could be used for pelleting. For this purpose, the reaction tubes were placed on a magnetic stand and then incubated at room temperature for 2 min. After incubation, the supernatant was removed, and the beads were washed with twice with 500 µL each of 80% ethanol (“Merck Millipore”, USA) and subsequently air-dried. Once the ethanol had evaporated, the samples were removed from the magnetic stand. The “SPRI Beads” were resuspended in the prescribed amount of “Low EDTA TE” buffer (Tab. A) and incubated at room temperature for 2 min. Lastly, the samples were re-placed on the magnetic stand. After ca. 2 min, complete separation of the supernatant and the “SPRI Beads” took place. The supernatant contained the purified product, was pipetted off and used for the next step.

TABLE A

Sample and reagent volumes for the purification steps with the “SPRI Beads”

| | | | |
|---|---|---|---|
| Step | Sample | “SPRI Beads” | “Low EDTA TE” buffer |
| Extension | 84 µL | 101 µL | 15 µL |
| Ligation | 30 µL | 36 µL | 20 µL |
| PCR | 50 µL | 40 µL | 20 µL |

### 1.1.2.4.2 Sequencing of WGBS Libraries

The sequencing of the WGBS libraries was done on the “NextSeq 500” platform (“Illumina”, USA) in the “TATAA-Biocenter” (Gothenburg, Sweden). This involved carrying out four 76 pair end (PE) runs in high-throughput mode.

### 1.1.2.5 Bioinformatic Evaluation of WGBS Results

The WGBS libraries could not be prepared using conventional protocols due to the high fragmentation and low amounts of zfDNA. The cfDNA libraries produced using the “Accel-NGS® Methyl-Seq DNA Library Kit” (“Swift Biosciences”, USA) therefore exhibited a different complexity and fragment distribution compared to conventional WGBS libraries. Therefore, a suitable bioinformatic evaluation pipeline also had to be established to be able to optimally analyze the data.

In general, multiple steps have to be established to be able to evaluate WGBS data (FIG. 1). First, the quality of the raw data is checked. For this, “FastQC” software (version 0.11.15, “Babraham Bioinformatics”, England) is most commonly used (see section 1.1.2.5.1). The software visualizes the quality of the sequencing, length distribution and composition of the reads. Furthermore, information about possible adapter contaminations as well as about number of kmers and PCR duplicates are provided. Kmers refer to sequences having a minimum length of two nucleotides that repeat again and again in the raw data.

If the quality control provides satisfactory results, trimming of the adapter sequences takes place. For the zfDNA libraries, the 10 nt long overhang generated by the “Adaptase” also had to be eliminated from the raw data (see section 1.1.2.5.2).

After trimming, the reads can be arranged against a reference genome of choice; this process is also referred to as alignment (see section 1.1.2.5.3). For alignment, there are many algorithms available. Depending on the nature of the WGBS Library, the appropriate one must be selected and optimized. For this purpose, mapping efficiency can be analyzed. This involves calculating what percentage of analyzed reads can be assigned to the reference genome. For conventional WGBS libraries, the “Bismark” algorithm is most commonly used (Krueger & Andrews [2011] Bioinformatics 27: 1571-1572). However, in the case of the cfDNA libraries described herein, “Bismark” (version 0.15.0, “Babraham Institute”, England) did not provide satisfactory results (mapping efficiency of approx. 70%). Therefore, further algorithms were tested.

The best results with a mapping efficiency of at least 98% were provided by the “Segemehl” algorithm (version 0.2.0, “Interdisciplinary Centre for Bioinformatics, Leipzig University”, Germany) (Otto et al. [2012] Bioinformatics 28: 1698-1704).

After alignment, the data are filtered according to CpG context and the desired coverage (at least fourfold), e.g., with the “Bisulfite Analysis Toolkit” (version 0.1, “Interdisciplinary Centre for Bioinformatics, Leipzig University”, Germany), and are only then used for peak calling (see section 1.1.2.5.3). Coverage, also called sequencing depth, specifies how frequently a position was read during sequencing. For example, an average coverage of 100-fold states that each sequenced base was read on average 100 times. Peak calling is the actual step in which the methylation status of the particular CpG is calculated. This involves looking at all reads which contain a certain CpG, calculating the ratio of cytosine to uracil, and outputting the result as a number between 0 and 1, wherein 0 corresponds to a methylation of 0% and 1 to a methylation of 100%.

Conventional libraries have an average coverage of 30 to 40-fold, which is also what the conventional methods for peak calling are designed to do. The zfDNA libraries had an average coverage of 8 to 10-fold due to their lower complexity.. Accordingly, filtering and peak calling had to be optimized, e.g. with the “Bisulfite Analysis Toolkit”..

Once the DNA methylation rates are established, further specific analyses can be done in a programming language of choice depending on the question asked. For the analyses described herein, “R” (version 3.2.0, “R Foundation for Statistical Computing”, Austria), “Perl” (version 5.26.0, “The Perl Foundation”, USA) and “Python” (version 3.3.6, “Python Software Foundation”, USA) were used (see section 1.1.2.5.3).

Since the analyses described herein required very high computing capacity, they were done on an “NEC HPC Linux Cluster”. The front-end processor was accessed via an SSH connection using “MobaXterm Personal Edition” software (“Mobatek”, France).

### 1.1.2.5.1 Quality Control of Raw Data

The raw data were provided in “FastQ” format. This is a text-based format which is used for storing of the reads as well as associated quality parameters. To check the quality of the sequencing, “FastQC” software was used.

### 1.1.2.5.2 Data Processing (Trimming)

The raw data were processed using “Cutadapt” software (version 1.9.1, “TU Dortmund”, Germany) (Martin EMBnet.journal 17). This involved carrying out two steps.
1.) Elimination of Overrepresented Sequences
During sequencing, the first 76 bases of each DNA fragment were read from both ends (76 PE sequencing). The libraries generated using the “Accel-NGS® Methyl-Seq DNA Library Kit” contained DNA fragments of differing length. This means that, if a DNA fragment was shorter than 152 bp, the “Illumina Adapters” or the flowcell were sequenced as well. This resulted in the presence of “NNNNNNNNNNN” sequences. Since in the further course of the data analysis the alignment of the associated and otherwise good quality reads would be prevented for this reason, the overrepresented sequences had to be removed. The command used for this purpose was:

cutadapt -q 20 -o 5 --minimum-length 30 -a GATCGGAAGAG -A AGATCGGAAGAG -o
<Name_Read_ 1>.clipped.fastq.gz -p <Name_Read_2>.clipped.fastq.gz
<Name_Read_ 1>.fastq.gz <Name_Read_2>.fastq.gz &><Name>.clipping.stats

2.) Removal of the Overhang Generated by “Adaptase”
During the production of the WGBS library, use was made of the enyzme “Adaptase”, which generated an overhang of low complexity at the 3′ end of the second read. This region, like the overrepresented sequences, would interfere in later alignment and therefore had to be removed. The command was:

cutadapt --minimum-length 25 -u 11 -o <Name_Read_2>.clipped.trimmed.fastq.gz -p
<Name_Read_1>.clipped.trimmed.fastq.gz <Name_Read_2>.clipped.fastq.gz
<Name_Read_ 1>.clipped.fastq.gz

### 1.1.2.5.3 Evaluation of Processed Data

Subsequent data analysis was carried out using the “Bisulfite Analysis Toolkit” [201]. The function of this modularly constructed Software is depicted in FIG. 2.

Alignment was carried out against the “HG19” reference genome. Several algorithms were tested, but surprisingly the “Segemehl” algorithm provided the best results (cf. section 1.1.2.5). The algorithm is based on searching for an optimal hit in the reference genome (Hoffmann et al. [2009] PLoS Comput. Biol. 5: e1000502). The maximum permitted number of inaccuracies per read (e.g., insertions, deletions, point mutations) was 10%. All hits which fell short of this threshold value were admitted to semiglobal alignment. Ultimately, only the reads with an accuracy of at least 90% were listed in a final file and used for further analyses.

The “BAM” format preferably used in this context is a compressed version of the “SAM” file, a text-based format which is generated by the algorithm for storing of results of the alignment. Mapping efficiency was statistically evaluated using, e.g., the “BAT_mapping_stat” module (Kretzmer et al. [2017] F1000Res. 6: 1490).

Lastly, all reads which belonged to a sample were merged into a “BAM” file using the “BAT_merging” module. Overlapping sequences were eliminated using the “ClipOverlap” (BamUtil version 1.0.13) module. The commands were:

perl BAT_mapping.pl -g hg19.fa -i hg19 -p <Name_Read_1>.clipped.trimmed.fastq.gz -q
<Name_Read_2>.clipped.trimmed.fastq.gz -t 16 -tmp <Folder> --segemehl segemehl.x -o
<Folder>/<Name>

perl BAT_mapping_stat.pl --bam <Name>.bam --fastq <Name>.clipped.trimmed.fastq.gz -b >
<Name>.stat

perl BAT_merging.pl -o <Name>.bam --bam <fiel_1>.bam,<file_2>.bam, ..., <file_n>.bam
bamUtil_1.0.13/bamUtil/bin/bam ClipOverlap --in <Name>.bam --out <Name>.nooverlap.bam

In the next step, DNA methylation was detected with the aid of “BAT_calling”. The module generates a “VCF” file. This is a text file which only contains information about the detected DNA methylation rates, coverage, number of covered nucleotides and the sequence context. In the further course of the analyses, this file was filtered for CpG context and coverage of at least eightfold. In this context, figures were generated and further “VCF” files as well as “BedGraph” files were generated. Next, the “BAT_summarize” module was used, which ascertained the mean values of detected DNA methylation rates of two groups. The calculated DNA methylation rates and the genomic coordinates of the cytosines were written into a text-based “BedGraph” file, which was used later on for the identification of differentially methylated regions.

The visualization of DNA methylation per group was carried out using the “BAT_overview” module [201]. The commands were:

BAT_calling.pl -d hg19.fa -q <Name>.nooverlap.bam --haarz segemehl_0_2_0/segemehl/
haarz.x -o <Folder>

BAT_filter_vcf.pl --vcf <Name>.nooverlap.vcf.gz --out <Name>_CG_cov_final --context CG --
MDP_min 8 --MDP_max 50

BAT_summarize.pl --in1 Adeno_CG_cov.bedgraph,PEKA_CG_cov.bedgraph --in2
Control_CG_cov.bedgraph -l cancer,control --h1 Adeno,PEKA --h2 Control --out pilot --cs
hg19.chrom.sizes --bgbw bedGraphToBigWig

Rscript BAT_overview.R -i pilot_cancer_control.txt -o pilot_overview.pdf -p cancer -q control

### 1.1.2.5.4 Correlation Analyses

In the context of this work, data from two methods for genome-wide examination of DNA methylation patterns were used: WGBS and methylation array (HM 450K).

“Bedtools” software was used for the correlation analyses. The “Bedtools Intersect” module reads both the WGBS results and the HM 450K results, checks them for overlap and writes the overlapping CpG loci into a new “BED” file. The “BED” format is a text file. Each line of the file contains genomic coordinates of a CpG. The columns are separated by a tab character. The “BED” file was subsequently directly loaded into “R” and subjected to “Pearson” correlation analysis (p-value < 0.01). The results were likewise visualized in R.

### 1.1.2.5.5 Selection of CpG Loci for the Plasma Panel

The WGBS data were evaluated as described. The “BedGraph” file generated using the “BAT_summarize” module contained three groups (control, adenocarcinoma, squamous cell carcinoma) having, in each case, 11 289 424 positions per group. The “BedGraph” file was divided into two lists. The first list contained 29 877 loci which showed differences in DNA methylation between the tumor and control groups. The second list contained 76,374 CpG loci differentially methylated in adenocarcinoma and squamous cell carcinoma groups, respectively. Differentially methylated referred to the regions which had a difference in DNA methylation of at least 15%.

Next, the two lists were sorted according to chromosomes and annotated with the “HG19” reference genome. The CpG loci which were located on chromosomes X, Y and M (mitochondrial chromosome) and within common SNPs (≥1% of the population) and were not protein-coding were discarded.

The remaining CpG loci had to meet one of the three criteria in order to be incorporated into the plasma panel:
1.) differentially methylated CpG was detected by both methods (WGBS and HM 450K),
2.) differentially methylated CpG lies within a cluster consisting of at least two further differentially methylated CpG loci; all CpG loci of the cluster are either hypo- or hypermethylated; the distance between the CpG loci is 2 to 20 nucleotides,
3.) it is a CpG with the highest differential DNA methylation rate (>0.8).

The DNA regions which met one of these three criteria were incorporated into the plasma panel (see Tab. 1). All calls used are described in detail below.

### 1.1.3 Further Components of the Plasma Panel (In Silico Data Analyses)

### 1.1.3.1 The Prognostic Study

In addition to diagnostically or therapeutically relevant information (e.g., stage and tumor entity), the panel should also contain prognostic information. Therefore, it was extended by 33 CpG loci, which were collected in the context of a clinical study. The title of the study was: “Comprehensive characterization of non-small cell lung cancer (NSCLC) by integrated clinical and molecular analysis”.

The HM 450K data set made available contained information about the DNA methylation status of a total of 41 lung carcinomas. The patients were classified according to survival time. In this context, 28 patients were included in the prognostically favorable group (survival longer than 15 months) and 13 in the unfavorable group (survival shorter than 13 months). The 33 CpG loci incorporated into the panel were able to separate both groups from one another on the basis of the DNA methylation pattern and thus contained information relevant for prognosis.

### 1.1.3.2 The Bivalent Chromatin Study

In addition to the WGBS and HM 450K results, 26 differentially methylated regions from the study on bivalent chromatin in tumors were incorporated into the plasma panel.

Bivalent promoters carry both activating and repressing histone modifications, which play an important role especially during cell differentiation processes. They are commonly incorrectly regulated in tumor cells. During the study, WGBS and HM 450K data sets of various tumor samples and cell lines (n=7000) were analyzed.

### 1.2 Methods: Validation of the Plasma Panel / Examination of Patient Samples

The set of methylation markers according to the invention, the plasma panel, contained 630 differentially methylated regions (Tab. 1). It was synthesized by the company “Roche” (Switzerland) and shipped on dry ice. This was a custom synthesized, non-commercially available “SeqCap Epi Enrichment Kit” ( Roche, Switzerland). According to the manufacturer, the panel was suitable for the analysis of both tissue samples and circulating, cell-free DNA.

It was validated in the context of a pilot study. For this purpose, blood plasma from 12 patients was provided by the DZL. Of these, three patients were healthy or tumor-free at the time of examination (control group) and nine were suffering from non-small cell lung carcinomas of different stages (tumor group).

Validation was carried out in multiple steps. First, the validation material, the circulating, cell-free DNA, was prepared. Extraction from plasma, quantification, quality control (QC) and bisulfite conversion were carried out as already described in sections 1.1.2.1-1.1.2.3.

Each 10 ng of converted zfDNA was then used for library preparation. Library preparation was done in two steps. In the first step, as described in section 1.1.2.4, a WGBS Library was prepared from each sample, which contained information about the entire zfDNA methylome of the corresponding patient. However, since only the 638 differentially methylated regions were to be sequenced and analyzed in the further course, they were extracted from the entire methylome and enriched in the second step. This was done using the “SeqCap Epi Enrichment Kit”, of which the plasma panel synthesized by “Roche” was a component (see section 1.2.1).

The finished library was subjected to a QC and was quantified (see section 1.1.2.2) and subsequently sequenced on the “MiSeq” (“Illumina”, USA) (see section 1.2.2). The sequencing data were stored in “FastQ” format and had to be subsequently analyzed (see section 1.2.3). For this purpose, the bioinformatic pipeline from section 1.1.2.5 was adapted, since this time only the 638 specific regions of the plasma panel were to be analyzed rather than the entire methylome.

The results were lastly used to develop a classifier, which subsequently interpreted the DNA methylation patterns and provided diagnostically as well as clinically relevant information about the health status of a patient (see section 1.2.3.3).

The same principle can be used to analyze samples from a patient who is to be diagnosed with lung tumors. Here, the samples are, however, not pooled for analysis.

### 2.2.1 Enrichment of Differentially Methylated Regions

The “SeqCap Epi Enrichment Kit” was used to extract and enrich 630 differentially methylated regions from the whole cfDNA methylome. One of the components of the kit was the designed plasma panel (see Tab. 1). The oligonucleotides contained therein, also called “Capture Probes”, hybridized to the differentially methylated regions and could be enriched and amplified in the further course (FIG. 3).

### Hybridization Reaction

The 12 WGBS libraries produced were pooled equimolarly within the different groups and were first prepared for a hybridization reaction. In the case of diagnostic samples, either individual samples are hybridized or pools of samples, each provided with a “Barcode”, are used. For this purpose, 1 µg of the WGBS library pool with 10 µL of “Bisulfite Capture Enhancer”, 1 µL of “SeqCap HE Universal Oligo” and 1 µL of “SeqCap HE Index Oligo” were pipetted into a 1.5 mL reaction vessel having a small hole in the lid. The sample was evaporated in a vacuum concentrator until a clear white pellet could be seen. The “SeqCap HE Universal” and “SeqCap HE Index” oligonucleotides were added in excess (1 µL corresponded to 1000 pmol) and served to bind the exposed WGBS universal and index adapters. Thus, the WGBS adapters should be prevented from interfering with the subsequent hybridization reaction.

For the actual hybridization reaction, 7.5 µL of two times “Hybridisation Buffer” and 3 µL of “Hybridisation Component A” were directly added to the pellet, mixed for 10 s, briefly centrifuged and incubated at 95° C. for 10 min. Then, the sample was transferred into a 0.2 µL reaction vessel, admixed with 4.5 µL of “Capture Probes”, mixed well and incubated in a thermal cycler at 47° C. for 72 h. The lid of the thermal cycler was preheated to 57° C. The “Capture Probes” were specifically synthesized for this project. They contained 638 different oligonucleotides which were complementary to the examined differentially methylated regions (see Tab. 1) and specifically bound them in the course of the hybridization reaction.

### Enrichment and Washing of Hybridized “Capture Probes”

In the next step, the bound “Capture Probes” were enriched and washed multiple times. For this purpose, multiple wash buffers as well as the “Capture Beads” were prepared according to the manufacturer’s instructions.

The hybridized sample was admixed with 100 µL of “Capture Beads”, briefly mixed and incubated in the thermal cycler at 47° C. for 45 min. The lid of the thermal cycler was preheated to 57° C. To prevent the beads from settling, the samples were briefly removed from the thermal cycler every 15 min and mixed. The “Capture Beads” used herein were streptavidin beads, which interacted with the biotinylated “Capture Probes”.

After incubation, the samples were removed from the thermal cycler and the “Capture Beads” were subjected to multiple wash steps. Separation of the beads from the buffer was performed each time at room temperature using the “DynaMagTM-PCR” magnet (“Thermo Fisher Scientific”, USA).

In the first part of the wash protocol, only buffers previously preheated to 47° C. were used. In this case, the sample was admixed with 100 µL of simple “Wash Buffer I”, briefly mixed, and pelleted with the aid of a magnet. The supernatant was discarded and the beads were dissolved in 200 µL of simple “Stringent Wash Buffer”, incubated in a thermal cycler at 47° C. for 5 min, and again pelleted with the aid of a magnet. The supernatant was again discarded and the beads were washed two further times with 200 µL of simple “Stringent Wash Buffer”.

The second part of the wash protocol took place completely at room temperature; accordingly, the buffers used for this had to be preheated to room temperature. First, the “Capture Beads” previously washed at 47° C. were dissolved in 200 µl of simple “Wash Buffer I”, mixed for 2 min, and pelleted with the aid of a magnet. The supernatant was discarded, the beads were admixed with 200 mL of simple “Wash Buffer II”, mixed for 1 min, and again pelleted with the aid of a magnet. Here too, the supernatant was discarded, the beads were dissolved in 200 mL of “Wash Buffer III”, briefly mixed, and lastly separated from the supernatant on the magnet.

For the subsequent elution, 50 µL of dH₂O were directly added to the beads, the beads were incubated at room temperature for 2 min and pelleted with the aid of a magnet. The supernatant was carefully pipetted from the reaction vessel and was used for all further steps.

### Amplification of the Enriched Differentially Methylated Regions

After washing, the enriched differentially methylated regions were amplified. For this purpose, 25 µL of two times “KAPA HiFi HotStart Ready Mix” (“Roche”, Switzerland) and 5 µL of “Post LM PCR Oligonucleotides” (“Roche”, Switzerland) were added, e.g., to 20 µL of eluate, mixed well and amplified in the thermal cycler with preheated lid using the following PCR program:
Step 1: 98° C. for 45 s
Step 2: 98° C. for 15 s
Step 3: 60° C. for 30 s
Step 4: 72° C. for 30 s
Step 5: Repetition of steps 1-4 for 15 more times
Step 6: 72° C. for 60 s
Step 7: Pause at 4° C.

### Purification of Enriched and Amplified Differentially Methylated Regions

The amplified regions were subsequently purified, e.g., using the “AmpureXP” beads (“Beckman Coulter”, USA). For this purpose, the beads were first preheated to room temperature. The sample was transferred into a 1.5 mL reaction vessel. 50 µL of dH₂O and 180 µL of “AmpureXP” beads were added to 50 µL of sample. The sample was briefly mixed, incubated at room temperature for 15 min, briefly centrifuged, and placed on the “DynaMag™-2” magnet (“Thermo Fisher Scientific”, USA). The supernatant was discarded and the beads were washed two times with each 200 µL of freshly prepared 80% ethanol. Then, the beads were dried at room temperature for 15 min. To elute the libraries, 52 µL of dH₂O were pipetted onto the dry beads. The beads were mixed well, incubated at room temperature for 2 min, and again placed on the “DynaMag™-2”. The supernatant was carefully pipetted off and was used for quantification, QC (see section 1.1.2.2) and sequencing on the “MiSeq”.

### 1.2.2 Sequencing of the Plasma Panel

Sequencing of the NGS library of enriched, differentially methylated regions was carried out on the “MiSeq”.

For this purpose, the library produced was first diluted to 4 nM and denatured. Then, 5 µl of the 4 nM library were transferred into a 1.5 mL reaction vessel, admixed with 5 µL of 0.2 M NaOH, briefly mixed, centrifuged at 280 g for 1 min, and incubated at room temperature for 5 min. The denatured library was then admixed with 990 µL of “Buffer HT1” (“Illumina”, USA) and again mixed well. This yielded a 20 pM library which was subsequently diluted to 4 pM using “Buffer HT1” and admixed with 10% “PhiX” (“Illumina”, USA).

Lastly, a “MiSeq 150 V3” cassette (“Illumina”, USA) was loaded with the finished sample and sequenced in a 76 PE run.

### 1.2.3 Bioinformatic Evaluation of the Sequencing Data

### 1.2.3.1 Quality Control and Processing of Raw Data

As described in sections 1.1.2.5.1 and 1.1.2.5.2, the data were subjected to a “FastQC” analysis and subsequently processed.

### 1.2.3.2 Evaluation of Processed Data

As described in section 1.1.2.5.3, the processed data were aligned against the “HG19” reference genome using the “Segemehl” algorithm. PCR duplicates were removed using “Samtools” (version 1.3.1, “Wellcome Trust Sanger Institute”, England, “Broad Institute of MIT and Harvard”, USA). The command was:

samtools rmdup -S <Name>.bam <Name>_wo_dup.bam

The DNA methylation rates within the sequenced regions were calculated using the “BAT_calling” module and filtered using the “BAT_filter_vcf” module according to the CpG context and a coverage of at least eightfold (see section 1.1.2.5.3). Lastly, the data were annotated against the regions of the plasma panel. The calls were:

for i in *vcf.gz; do o=‘echo $i | sed ‘s/.vcf.gz/_CG.vcf.gz/’; echo $i $o; perl BAT_filter_vcf --vcf
$i --out $o --context CG; done
for i in *_CG.vcf.gz; do
o=‘echo $i | sed ‘s/_CG.vcf.gz/_CG.cov.region.vcf.gz/’
echo $i $o
zcat $i | grep “#” >tmp.vcf; bedtools intersect -u -b OID44445_hg19_07mar2017_primary_
targets.bed -a $i >>tmp. vcf
gzip tmp.vcf
perl BAT_filter_vcf --vcf tmp.vcf.gz --out $o --context CG --MDP_min 8 --MDP_max 200
rm tmp.vcf.gz
done
bedtools unionbedg -filler NA -header -names <sample_1> ... <sample_n> -i
<name_sample_1>_wo_dup_CG.cov.region.bedgraph ...
<name_sample_n>_wo_dup_CG.cov.region.
bedgraph > <name>.bed

### 1.2.3.3 Creation of a Classifier

The plasma panel was then used to analyze the DNA methylation pattern of a patient. From this, it was to be concluded whether a patient has a malignant lung tumor. If this is the case, information about the entity of the tumor and the prognosis of the patient affected was to be derived from the DNA methylation profile. This can be done on the basis of the correlation between the methylation patterns which are present in the patient and the methylation markers which are important according to the invention.

For this purpose, a classifier can be created which is capable of rapidly and reliably interpreting the results of the pipeline described in sections 1.2.3.1 and 1.2.3.2. A classifier, also called predictive modeling, is an example of supervised learning. It is the goal of a classifier, after receiving variables (e.g., DNA methylation patterns) and an annotation, to first create a model which is later capable of correctly classifying the variables of independent samples (FIG. 4).

The software “Qlucore Omics Explorer”, e.g., offers several possibilities of creating, using DNA methylation data, an optimal classifier for the particular question. For this, a selection from three algorithms can be made: “k-Nearest Neighbors Algorithm” (kNN), “Support Vector Machines” (SVM) and “Random Trees” (RT). For kNN, a class assignment is made based on the consideration of k nearest neighbors. SVM describes each object by a vector in a vector space. Within the vector space, a hyperplane is placed such that it acts as a separation plane between the groups and divides them into two classes. RT consists of multiple uncorrelated decision trees which were generated during the learning process. Each tree makes a decision, the class having the most votes ultimately decides on the final classification.

In general, it is difficult to predict in advance which algorithm will provide the optimal results for a new problem. Therefore, all three available algorithms were tested to find the best one for the particular category.

### 2. RESULTS

### 2.1 Results: “Development of the Plasma Panel”

### 2.1.1 Detection of Tumor– and Entity–Specific DNA Methylation in Primary Tumor Tissue

40 surgical preparations and corresponding controls were examined for their genome-wide DNA methylation using the “Illumina Infinium HumanMethylation450K BeadChip”.

In comparison with healthy lung tissue, 898 aberrantly methylated CpG loci were identified in malignant tumor tissue (q< 1×10⁻²³, σ/σₘₐₓ> 0.4; FIG. 5A). Adeno- and squamous cell carcinoma are the two most common entities of non-small cell lung carcinoma. One analysis yielded 1167 differentially methylated CpG loci among the tumor entities (FDR < 1 × 10⁻⁴; FIG. 5B).

In the following, those CpG loci were selected, which allowed reliable classification of lung tumors on the basis of malignancy and entity. For this purpose, the bioinformatic analyses described in section 1.1.1 were carried out, which yielded 287 CpG loci. Said loci were incorporated into a set of methylation markers preferred according to the invention, the plasma panel (Tab. 1).

### 2.1.2 Detection of Tumor– and Entity–Specific DNA Methylation in Blood Plasma

As described in section 1.1.2.2, each individual cell-free, circulating DNA sample was quantified and subjected to a strict quality control after extraction. The total amount of extracted DNA was 10 to 30 ng per sample, of which 1 ng was analyzed using the “Agilent 2100 Bioanalyzer”. The samples showed a clear peak at ca. 167 bp. The peaks at 35 bp and 10 380 bp corresponded to the bottom or top markers, respectively (not shown).

After bisulfite conversion, the cfDNA samples were used to produce WGBS libraries. The completed libraries were, in turn, quantified and subsequently subjected to a quality control using the “Agilent 2100 Bioanalyzer”. All samples showed a clear peak at ca. 300 bp and therefore met the requirements for sequencing.

The WGBS libraries produced were sent on dry ice to the “TATAA Biocenter”, where they were pooled and, depending on the sample sequenced with an average coverage of 8 to 10-fold on a “NextSeq 500” platform. The raw data were provided in “FastQ” format.

The quality of the raw data was checked using “FastQC” software. Since the samples were sequenced 76 PE, the read length was, as expected, 76 bp. Within a read, the content of adapters and of nonidentifiable signals was 0%. The accuracy of sequencing was specified in “Phred” values. Each “Phred” value describes how accurately nucleotide reads were made during the course of sequencing. The raw data had a “Phred” score of over 30, which corresponded to an accuracy of more than 99.9%. Furthermore, only a very small amount of kmers could be detected. Kmers refer to sequences having a minimum length of two nucleotides that repeat again and again in the raw data. The number of PCR duplicates was virtually 0%. The amount of PCR duplicates is ascertained by calculating the percentage of deduplicated sequences and comparing it with the number of all sequences. A small amount of kmers and PCR duplicates indicates good library and sequencing quality.

Furthermore, a WGBS-typical base composition was analyzed. During bisulfite conversion, most unmethylated cytosines were replaced by thymines. Therefore, the thymine content of the raw data was ca. 50% and the cytosine content was virtually 0%. The adenine and guanine compositions were not influenced during bisulfite conversion and were 25% each.

Subsequently, the WGBS raw data were processed using “Cutadapt” software (see section 1.1.2.5.2). The processing removed both overrepresented sequences and the 10 nt long overhang at the start of read 2.

The processed sequencing data were then loaded into the “Bisulfite Analysis Toolkit” and aligned against the “HG19” reference genome using the “Segemehl” algorithm implemented there. The efficiency of alignment is specified as mapping efficiency. This determines how much percent of reads can be assigned to the reference genome.. In this case, the mapping efficiency of the “Segemehl” algorithm was 98% to 99% and was therefore suitable for all further analyses.

Next, the alignments of the control, adenocarcinoma and squamous cell carcinoma groups were loaded into the “BAT_calling” module. The module ascertained DNA methylation rates of respective cytosines. The cytosines which lay within a CpG region and had a coverage of at least eightfold were then identified using the “BAT_filtering” module and used for all further analyses.

More than 4 million CpG loci per group met the criteria and were analyzed later on using the “BAT_overview” module. The results clearly showed that both the lung carcinoma group and the control group can be distinguished from one another on the basis of the DNA methylation patterns (FIG. 6A). Furthermore, genome-wide hypermethylation of the lung carcinoma groups compared to the control group is visible (FIG. 6A).

To detect the differentially methylated regions specific for the respective group, filtering was carried out according to a difference in DNA methylation of at least 15%. In this context, the number of differentially methylated CpG loci in the plasma of lung carcinoma patients was 18 000 (FIG. 7A). Furthermore, 44 000 CpG loci were identified which were differentially methylated depending on the entity in adeno- and squamous cell carcinoma patients (FIG. 7B). As described in section 1.1.2.5.5, said loci were subjected to further analyses and used to create the plasma panel. The completed set of methylation markers, i.e., the completed plasma panel, contained 630 differentially methylated regions (Tab. 1). Oligonucleotides which hybridize to these differentially methylated regions were synthesized as “Capture Probes” and thus represent means for diagnosing lung tumors.

### 2.1.3 Correlation Analyses of the Used Methods for Genome-Wide Detection of DNA Methylation Patterns

To compare the detected DNA methylation patterns in the surgical preparations with those in the blood plasma of the lung carcinoma patients, a “Pearson” correlation analysis was carried out using “R” and “Bedtools” (see section 1.1.2.5.4), which, depending on the sample, yielded a concordance of 71% to 77% (p-value < 2.2 × 10⁻¹⁶, FIG. 8).

This shows that results on the basis of surgical preparations or solid biopsies cannot be readily applied to liquid biopsies, so that the present validation with liquid biopsies is crucial for the validity of the diagnostic procedure.

### 2.2 Results Relating to “Validation of the Plasma Panel”

### 2.2.1 Creation of NGS Libraries

First, as described in section 1.1.2.2, the extracted cfDNA samples were quantified and subjected to a quality control. For this purpose, 1 ng of each sample was examined using the “Agilent 2100 Bioanalyzer”. All cfDNA samples used showed a clear peak at ca. 167 bp. Subsequently, the samples were bisulfite-converted and used to produce NGS libraries. As described in section 1.2.1, production of the libraries was performed in two steps.

In the first step, WGBS libraries which comprised information about the whole cfDNA methylome were produced. All 12 WGBS libraries produced showed a clear, large peak at ca. 300 bp. The larger 300 to 1,000 bp peaks were the so-called daisy chains, i.e., ssDNA fragments hybridized to each other. According to the manufacturer’s instructions, they neither influence the subsequent hybridization reaction nor the actual sequencing and therefore do not have to be eliminated.

In the second step, the WGBS libraries produced were quantified, equimolarly pooled, and processed using the “SeqCap Epi Enrichment Kit”. The kit used herein contained the so-called “Capture Probes” which were specifically synthesized for this purpose. The “Capture Probes” specifically hybridize to the 638 regions of the plasma panel (see Tab. 1). After hybridization, the “Capture Probes” together with the bound differentially methylated regions were enriched, washed and amplified. The amplified library was then quantified and subjected to a quality control (e.g., “Agilent 2100 High Sensitivity DNA Kit”). The finished library had a high peak at ca. 300 bp and therefore met the sequencing requirements of the “MiSeq”.

### 2.2.2 Sequencing and Data Analysis

First, sequencing was optimized on the “MiSeq”. Sequencing was done in a 76 PE mode. Thus, the first 76 bp of the sequenced DNA fragments were read from both ends. To achieve the optimal cluster density, the library was diluted to 4 pM. The libraries described herein were unbalanced. Unbalanced refers to libraries, whose AT or GC concentration is less than 40% or more than 60%. Because of their composition, such libraries usually have an unsatisfactory sequencing quality. To prevent this, the library can be admixed with “PhiX Control V3”. The concentration of “PhiX” must be individually adapted depending on the library. The optimal concentration of “PhiX Control V3” was 10% in the present case.

After sequencing, the data were stored in “FastQ” format. The quality of the raw data was checked using “FastQC” software.

Because of 76 PE sequencing, the read length was 76 bp. The content of adapters and nonidentifiable signals within a read was 0%. The raw data had a “Phred” score of over 30, which corresponded to a sequencing accuracy of more than 99.9%. The base composition (thymine content at ca. 50%, cytosine content at virtually 0%, adenine and guanine content at 25%) indicated successful bisulfite conversion. The first 10 nt of the second read was an overhang generated by the enzyme “Adaptase”. The deviation of the experimentally ascertained GC content from the theoretically calculated one was also because of the bisulfite conversion.

The number of PCR duplicates was ca. 15%. The number of deduplicated sequences deviated greatly from the total amount. However, this is not unusual for a panel. In contrast to a genome-wide sequencing, in a panel only a small region of the genome is sequenced. This leads to a very low complexity of the library and, accordingly, to the formation of PCR duplicates. The number of kmers is very low and does not interfere with further evaluation.

In summary, it can be stated that the panel sequencing data had a very good quality. To process the data, two steps were carried out. First, the 10 nt long overhang at the start of read 2 and adapters were removed using “Cutadapt” software. Then, the PCR duplicates were completely eliminated using “Samtools” software.

The processed sequencing data were then loaded into the “Bisulfite Analysis Toolkit”. Alignment was carried out using “Segemehl” against the “HG19” reference genome. The mapping efficiency was at least 90%. This means that at least 90% of the raw data could be assigned to the reference genome. The average coverage, i.e., the sequencing depth, was 10- to 30-fold depending on the sample.

In the next step, DNA methylation was to be detected. For this purpose, the 12 alignments were loaded into the “BAT_calling” module. The positions ascertained were then first annotated against the “HG19” reference genome using “Bedtools”. Then, the methylated positions were filtered according to a coverage of at least eightfold using the “BAT_filtering” module. Furthermore, the module for creating a classifier was used to select only those positions that were, on the one hand, located in a CpG region and, on the other hand, were listed in the plasma panel (Tab. 1).

### 2.2.3 Creation of a Classifier

The ascertained cfDNA methylation rates were used to create a classifier. As described in section 1.2.3.3, “Qlucore Omics Explorer” software was used for this purpose, which contained the following classification algorithms: “k-Nearest Neighbors Algorithm” (kNN), “Support Vector Machines” (SVM) and “Random Trees” (RT).

The plasma panel was designed such that it should be optimally capable of providing information regarding the malignancy, the entity and the stage of a tumor. These questions could be answered reliably by the choice of a suitable classifier. Furthermore, it should also be possible to obtain information relating to prognosis.

To assess a classifier, two parameters were considered: accuracy and complexity. The accuracy of a classifier was specified in values between 0 and 1, wherein 0 corresponded to an accuracy of 0% and 1 to an accuracy of 100%. Complexity indicated how many differentially methylated positions or markers had to be analyzed so that the classifier achieved this accuracy. The fewer markers that needed to be evaluated, the more appropriate the classifier was for the clinic. This is because the error rate, time and costs of the method increase with the number of positions to be analyzed.

The first question was whether a patient was suffering in general from a malignant lung tumor. For this purpose, both the kNN algorithm and the RT algorithm provided an accuracy of 100%. For classification, the RT algorithm required 237 differentially methylated positions present in the panel. The kNN, on the other hand, only 10 positions, which qualified it as optimal for this problem (FIG. 9A).. Stronger methylation is found in tumor tissue at 9 of the 10 positions, a weaker methylation at one.

The question regarding entity could be answered by all three algorithms with an accuracy of 100%. For the calculations, kNN required 22 positions, SVM 22 positions and RT 10 positions. Therefore, the RT algorithm was best-suited for this question (FIG. 9B), but also the other algorithms can be used. For all the markers evaluated, there is a stronger methylation in the case of adenocarcinoma than in the case of squamous cell carcinoma.

For the last question of tumor stage, it was most difficult to choose a suitable classifier. Using 523 positions, the SVM algorithm managed to distinguish the late tumor stages with 80% accuracy (FIG. 9C). Thereby, the evaluated positions are partly more methylated in the early, partly in the late stages..

All positions and classification parameters are described in detail in the annex (see Tab. 2-4). The described results therefore render it possible to carry out a diagnosis of lung cancer from a liquid biopsy of a patient by means of sequencing of purified, bisulfite-converted DNA enriched via oligonucleotides which hybridize to the methylation markers. In this case, the sequencing data are preferably aligned against a reference genome using the Segemehl algorithm and then evaluated on the basis of the correlation of the methylation, optionally on the basis of the classification as described above.

### 3.1 Further Information on Development and Validation of the Plasma Panel Selection of CpG Loci for the Plasma Panel

### A. Filtering According to Chromosome

Chromosomes M, X and Y were discarded; the commands were:

grep -v “chrM” <Name>.bedgraph | grep -v “chrX” | grep -v “chrY” >
<Name>.ohneMXY.bedgraph
cut -f1 <Name>ohneMXY.bedgraph | sort | uniq

### B. Annotation With the “HG19” Reference Genome

less gencode.v19.only.genes.bed | perl -ane ‘if($F[5] eq “+”){$F[1]=$F[1]-1500}else{$F[2]
=$F[2]+ 1500}; print “$F[0]\t$F[1]\t$F[2]\t$F[3]\t$F[4]\t$F[5]\n’” >
gencode. v19.only.genes.TSS_1500nt.bed
bedtools intersect -wa -wb -a <Name>ohneMXY.bedgraph -b gencode.v19.only.genes.TSS_
1500nt.bed

### C. Selection of the CpG Loci Detected by WGBS and HM 450K

bedtools intersect -wa -wb -a <WGBS_data>.bedgraph -b <450K_BeadChip_data>.bed | perl -
ane ‘if(($F[3]>0 && $F[7]>0) || ($F[3]<0 && $F[7]<0)){print $_}’ >
overlap_WGBS_450K_BeadChip.bed
bedtools intersect -wa -wb -a overlap_WGBS_450K_BeadChip.bed -b
gencode.v19.only.genes. TSS_1500nt.bed | cut -f1-4,8,9,13 >
overlap_WGBS_450K_BeadChip_gencode.v19.bed

### D. Selection of Differentially Methylated CpG Clusters

For this, CpG loci which lay within a cluster consisting of at least two further differentially methylated CpG loci were selected. All CpG loci of the cluster were either hypomethylated or hypermethylated. The distance between the CpG loci was 2 to 20 nt.

less <Name>ohneMXY.bedgraph | sort -k10, 10 | bedtools groupby-g 7,8,9,10,11,12 -c 1,2,3,1
-o collapse,collapse,collapse,count | perl -ane ‘if($F[-1]>=3){print $_}’ | perl -ane
‘@chr=split(/,/,$F[6]); @start=split(/,/,$F[7]); @end=split(/,/,$F[8]); for($i=0; $i<$F[-1]; $i++){print
“$chr[$i]\t$start[$i]\t$end[$i]\ t$F[0]\t$F[1]\t$F[2]\t$F[3]\t$F[4]\t$F[5]\n”}’ > < Name>ohneMXY
_mind3CpG_annotation.bedgraph
perl CpG_cluster_Swetlana --min 2 --max 20 --in
<Name>ohneMXY_mind3CpG_annotation.bedgraph | grep protein >
<Name>ohneMXY_mind3CpG_3diffCpG.bedgraph
less <Name>ohneMXY_mind3CpG_3diffCpG.bedgraph | bedtools groupby -g 7,11 -c 3,1,2,3
-o collapse,distinct,min,max | perl -ane ‘print “$F[3]\t$F[4]\t$F[5]\t$F[2];$F[0]\n”’ >
<Name>ohneMXY_mind3CpG_3diffCpG_sortiert.bedgraph
bedtools intersect -wa -wb -a <Name>ohneMXY_mind3CpG_3diffCpG _sortiert.bedgraph -b
<Diff_fiel>.bedgraph | bedtools groupby -g 1,2,3,4 -c 8 -o mean | perl -ane ‘$a=abs($F[4]);
chomp $_; print “$_\t$a\n”’ | sort -k6,6n | tail -150 >
<Name>ohneMXY_mind3CpG_3diffCpG_sortiert_beste_150_regionen.bedgraph

### E. Selection of Positions Having the Highest Differential DNA Methylation

bedtools intersect -v -a <Name>ohneMXY.bedgraph -b
<Name>ohneMXY_mind3CpG_3diffCpG_sortiert_beste_150_regionen.bedgraph | bedtools
intersect -wa -wb -a stdin -b gencode. v19.only .genes. TSS_1500nt_ohnechrM.bed | grep
protein | perl -ane 𔃶$a=abs($F[5]); chomp $_; print “$_\t$a\n”’ | sort -V -k13,13n | cut -
f1,2,3,10,13 | tail -100 > <Name>ohneMXY_die_besten_einzel_ cpg.bedgraph

Table 1: Set of methylation markers (plasma panel; 630 differentially methylated regions). The column “Tumor” indicates whether an increased (hypermethylated) or reduced (hypomethylated) methylation was identified in tumor tissue. A. 350 regions which detect a malignant lung tumor. B. 247 regions which distinguish the most common lung carcinoma entities (adenocarcinoma and squamous cell carcinoma) from one another. C. 33 prognostically relevant CpG loci. Method: cfDNA (WBGS): cfDNA or surgical preparations (HM 450 K): surgical; the bivalent chromatin study: bChrSt.

| A. Lung carcinoma or lung tissue? | | | | Tumor |
|---|---|---|---|---|
| Chromosome | Start | End | Method | |
| chr1 | 57955028 | 57955174 | cfDNA, surgical | hypomethylated |
| chr1 | 193191311 | 193191476 | cfDNA, surgical | hypermethylated |
| chr10 | 85985699 | 85985859 | cfDNA, surgical | hypomethylated |
| chr10 | 110084584 | 110084739 | cfDNA, surgical | hypomethylated |
| chr10 | 130860130 | 130860266 | cfDNA, surgical | hypomethylated |
| chr11 | 57798784 | 57798925 | cfDNA, surgical | hypomethylated |
| chr11 | 57948628 | 57948769 | cfDNA, surgical | hypomethylated |
| chr11 | 58034333 | 58034464 | cfDNA, surgical | hypomethylated |
| chr11 | 59634150 | 59634282 | cfDNA, surgical | hypomethylated |
| chr11 | 59824464 | 59824610 | cfDNA, surgical | hypomethylated |
| chr11 | 131547241 | 131547390 | cfDNA, surgical | hypomethylated |
| chr12 | 7818556 | 7818707 | cfDNA, surgical | hypomethylated |
| chr12 | 111016497 | 111016626 | cfDNA, surgical | hypomethylated |
| chr12 | 128899218 | 128899363 | cfDNA, surgical | hypomethylated |
| chr14 | 58064847 | 58064987 | cfDNA, surgical | hypomethylated |
| chr14 | 88621354 | 88621491 | cfDNA, surgical | hypomethylated |
| chr15 | 63349114 | 63349271 | cfDNA, surgical | hypermethylated |
| chr15 | 87516105 | 87516269 | cfDNA, surgical | hypomethylated |
| chr16 | 20055126 | 20055299 | cfDNA, surgical | hypomethylated |
| chr16 | 34255462 | 34255596 | cfDNA, surgical | hypomethylated |
| chr17 | 46799562 | 46799708 | cfDNA, surgical | hypermethylated |
| chr2 | 2019860 | 2020000 | cfDNA, surgical | hypomethylated |
| chr2 | 66671403 | 66671543 | cfDNA, surgical | hypermethylated |
| chr2 | 118569132 | 118569281 | cfDNA, surgical | hypomethylated |
| chr2 | 155089787 | 155089940 | cfDNA, surgical | hypomethylated |
| chr20 | 29960903 | 29961067 | cfDNA, surgical | hypomethylated |
| chr21 | 31987899 | 31988061 | cfDNA, surgical | hypomethylated |
| chr3 | 159175958 | 159176133 | cfDNA, surgical | hypomethylated |
| chr4 | 77703312 | 77703460 | cfDNA, surgical | hypomethylated |
| chr5 | 5033914 | 5034062 | cfDNA, surgical | hypomethylated |
| chr5 | 5568513 | 5568662 | cfDNA, surgical | hypomethylated |
| chr5 | 141130550 | 141130698 | cfDNA, surgical | hypomethylated |
| chr6 | 5132810 | 5132954 | cfDNA, surgical | hypermethylated |
| chr6 | 20877268 | 20877408 | cfDNA, surgical | hypomethylated |
| chr6 | 27648240 | 27648385 | cfDNA, surgical | hypermethylated |
| chr6 | 55956239 | 55956395 | cfDNA, surgical | hypomethylated |
| chr7 | 149112327 | 149112464 | cfDNA, surgical | hypermethylated |
| chr8 | 54798658 | 54798811 | cfDNA, surgical | hypomethylated |
| chr1 | 2198804 | 2198961 | surgical | hypermethylated |
| chr1 | 6515521 | 6515702 | surgical | hypermethylated |
| chr1 | 6520115 | 6520257 | surgical | hypermethylated |
| chr1 | 19764609 | 19764757 | surgical | hypermethylated |
| chr1 | 34642324 | 34642455 | surgical | hypermethylated |
| chr1 | 47694840 | 47694995 | surgical | hypermethylated |
| chr1 | 50883315 | 50883461 | surgical | hypermethylated |
| chr1 | 50886707 | 50886857 | surgical | hypermethylated |
| chr1 | 50886870 | 50887021 | surgical | hypermethylated |
| chr1 | 79472375 | 79472516 | surgical | hypermethylated |
| chr1 | 110610821 | 110610964 | surgical | hypermethylated |
| chr1 | 110611386 | 110611542 | surgical | hypermethylated |
| chr1 | 110611971 | 110612108 | surgical | hypermethylated |
| chr1 | 119522559 | 119522707 | surgical | hypermethylated |
| chr1 | 150595130 | 150595282 | surgical | hypermethylated |
| chr1 | 153896523 | 153896648 | surgical | hypomethylated |
| chr1 | 155162673 | 155162808 | surgical | hypomethylated |
| chr1 | 158324396 | 158324540 | surgical | hypomethylated |
| chr1 | 158549201 | 158549351 | surgical | hypomethylated |
| chr1 | 158575697 | 158575854 | surgical | hypomethylated |
| chr1 | 158736216 | 158736378 | surgical | hypomethylated |
| chr1 | 159284004 | 159284160 | surgical | hypomethylated |
| chr1 | 159284209 | 159284363 | surgical | hypomethylated |
| chr1 | 159682419 | 159682564 | surgical | hypomethylated |
| chr1 | 160782978 | 160783141 | surgical | hypomethylated |
| chr1 | 161008634 | 161008907 | surgical | hypomethylated |
| chr1 | 166039366 | 166039510 | surgical | hypomethylated |
| chr1 | 175050401 | 175050549 | surgical | hypomethylated |
| chr1 | 182025968 | 182026117 | surgical | hypermethylated |
| chr1 | 223948836 | 223948969 | surgical | hypermethylated |
| chr1 | 248903024 | 248903175 | surgical | hypomethylated |
| chr10 | 15688934 | 15689073 | surgical | hypomethylated |
| chr10 | 34405682 | 34405834 | surgical | hypermethylated |
| chr10 | 44285786 | 44285947 | surgical | hypomethylated |
| chr10 | 98129672 | 98129823 | surgical | hypomethylated |
| chr10 | 98129826 | 98129981 | surgical | hypomethylated |
| chr10 | 102894966 | 102895098 | surgical | hypermethylated |
| chr10 | 104000754 | 104000901 | surgical | hypermethylated |
| chr10 | 118892505 | 118892640 | surgical | hypermethylated |
| chr10 | 118893055 | 118893205 | surgical | hypermethylated |
| chr10 | 121075240 | 121075380 | surgical | hypomethylated |
| chr10 | 134598276 | 134598414 | surgical | hypermethylated |
| chr11 | 627096 | 627254 | surgical | hypermethylated |
| chr11 | 31826508 | 31826642 | surgical | hypermethylated |
| chr11 | 40136733 | 40136880 | surgical | hypomethylated |
| chr11 | 40312591 | 40312717 | surgical | hypomethylated |
| chr11 | 57005866 | 57005971 | surgical | hypomethylated |
| chr11 | 57006196 | 57006350 | surgical | hypomethylated |
| chr11 | 59270333 | 59270463 | surgical | hypomethylated |
| chr11 | 68166958 | 68167099 | surgical | hypermethylated |
| chr11 | 69061832 | 69061978 | surgical | hypomethylated |
| chr11 | 75831643 | 75831777 | surgical | hypermethylated |
| chr11 | 86085859 | 86085993 | surgical | hypermethylated |
| chr11 | 123885618 | 123885776 | surgical | hypomethylated |
| chr11 | 133005846 | 133005990 | surgical | hypomethylated |
| chr12 | 5918113 | 5918249 | surgical | hypomethylated |
| chr12 | 21590167 | 21590318 | surgical | hypomethylated |
| chr12 | 50665695 | 50665835 | surgical | hypermethylated |
| chr12 | 54423481 | 54423625 | surgical | hypermethylated |
| chr12 | 54448654 | 54448816 | surgical | hypermethylated |
| chr12 | 54448836 | 54448981 | surgical | hypermethylated |
| chr12 | 56329564 | 56329709 | surgical | hypermethylated |
| chr12 | 62584958 | 62585102 | surgical | hypermethylated |
| chr12 | 75601386 | 75601538 | surgical | hypermethylated |
| chr12 | 114847503 | 114847664 | surgical | hypermethylated |
| chr12 | 126142819 | 126142966 | surgical | hypomethylated |
| chr12 | 129595318 | 129595466 | surgical | hypomethylated |
| chr13 | 41593317 | 41593485 | surgical | hypomethylated |
| chr13 | 42188553 | 42188701 | surgical | hypermethylated |
| chr13 | 58207783 | 58207923 | surgical | hypermethylated |
| chr14 | 21623728 | 21623873 | surgical | hypomethylated |
| chr14 | 37128511 | 37128658 | surgical | hypermethylated |
| chr14 | 55907221 | 55907370 | surgical | hypermethylated |
| chr14 | 57274684 | 57274828 | surgical | hypermethylated |
| chr14 | 57275089 | 57275229 | surgical | hypermethylated |
| chr14 | 57275889 | 57276137 | surgical | hypermethylated |
| chr14 | 57276179 | 57276336 | surgical | hypermethylated |
| chr14 | 57276449 | 57276590 | surgical | hypermethylated |
| chr14 | 57277149 | 57277295 | surgical | hypermethylated |
| chr14 | 57278109 | 57278251 | surgical | hypermethylated |
| chr14 | 57284449 | 57284596 | surgical | hypermethylated |
| chr14 | 60977778 | 60977928 | surgical | hypermethylated |
| chr14 | 60978086 | 60978221 | surgical | hypermethylated |
| chr14 | 77769608 | 77769754 | surgical | hypomethylated |
| chr15 | 42749674 | 42749956 | surgical | hypermethylated |
| chr15 | 45409243 | 45409393 | surgical | hypermethylated |
| chr15 | 72520560 | 72520691 | surgical | hypomethylated |
| chr15 | 86233150 | 86233290 | surgical | hypermethylated |
| chr15 | 89920745 | 89920964 | surgical | hypermethylated |
| chr15 | 89922266 | 89922403 | surgical | hypermethylated |
| chr16 | 23850031 | 23850175 | surgical | hypomethylated |
| chr16 | 29086204 | 29086356 | surgical | hypermethylated |
| chr16 | 31580915 | 31581053 | surgical | hypermethylated |
| chr16 | 48592619 | 48592755 | surgical | hypermethylated |
| chr16 | 59789141 | 59789301 | surgical | hypomethylated |
| chr16 | 59790110 | 59790246 | surgical | hypomethylated |
| chr16 | 66613021 | 66613174 | surgical | hypermethylated |
| chr16 | 66613201 | 66613354 | surgical | hypermethylated |
| chr16 | 76342543 | 76342697 | surgical | hypomethylated |
| chr17 | 750165 | 750314 | surgical | hypermethylated |
| chr17 | 31689711 | 31689863 | surgical | hypomethylated |
| chr17 | 32613223 | 32613361 | surgical | hypomethylated |
| chr17 | 35299524 | 35299661 | surgical | hypermethylated |
| chr17 | 55951984 | 55952129 | surgical | hypermethylated |
| chr17 | 59532229 | 59532369 | surgical | hypermethylated |
| chr17 | 67536233 | 67536383 | surgical | hypomethylated |
| chr17 | 72619477 | 72619639 | surgical | hypomethylated |
| chr18 | 20714264 | 20714392 | surgical | hypomethylated |
| chr18 | 21596836 | 21596981 | surgical | hypomethylated |
| chr18 | 61143869 | 61144219 | surgical | hypomethylated |
| chr18 | 61144261 | 61144399 | surgical | hypomethylated |
| chr19 | 9609321 | 9609462 | surgical | hypermethylated |
| chr19 | 18761488 | 18761632 | surgical | hypermethylated |
| chr19 | 19625186 | 19625348 | surgical | hypermethylated |
| chr19 | 42600201 | 42600339 | surgical | hypermethylated |
| chr19 | 48285227 | 48285396 | surgical | hypermethylated |
| chr19 | 53038895 | 53039056 | surgical | hypermethylated |
| chr2 | 2336353 | 2336494 | surgical | hypomethylated |
| chr2 | 3642551 | 3642688 | surgical | hypermethylated |
| chr2 | 43496147 | 43496286 | surgical | hypermethylated |
| chr2 | 45171739 | 45171891 | surgical | hypermethylated |
| chr2 | 45232352 | 45232491 | surgical | hypermethylated |
| chr2 | 63280990 | 63281212 | surgical | hypermethylated |
| chr2 | 63281305 | 63281462 | surgical | hypermethylated |
| chr2 | 63282625 | 63282788 | surgical | hypermethylated |
| chr2 | 63282935 | 63283081 | surgical | hypermethylated |
| chr2 | 63283888 | 63284202 | surgical | hypermethylated |
| chr2 | 73021274 | 73021424 | surgical | hypermethylated |
| chr2 | 100516804 | 100516939 | surgical | hypomethylated |
| chr2 | 105069122 | 105069275 | surgical | hypomethylated |
| chr2 | 105086941 | 105087083 | surgical | hypomethylated |
| chr2 | 124920570 | 124920719 | surgical | hypomethylated |
| chr2 | 127453687 | 127453859 | surgical | hypomethylated |
| chr2 | 162280362 | 162280605 | surgical | hypermethylated |
| chr2 | 176964058 | 176964200 | surgical | hypermethylated |
| chr2 | 176964383 | 176964599 | surgical | hypermethylated |
| chr2 | 176964651 | 176964790 | surgical | hypermethylated |
| chr2 | 176980760 | 176980908 | surgical | hypermethylated |
| chr2 | 176980985 | 176981133 | surgical | hypermethylated |
| chr2 | 176982263 | 176982420 | surgical | hypermethylated |
| chr2 | 176986185 | 176986321 | surgical | hypermethylated |
| chr2 | 176988868 | 176989016 | surgical | hypermethylated |
| chr2 | 176989280 | 176989410 | surgical | hypermethylated |
| chr2 | 177014478 | 177014625 | surgical | hypermethylated |
| chr2 | 177014869 | 177015014 | surgical | hypermethylated |
| chr2 | 177027372 | 177027510 | surgical | hypermethylated |
| chr2 | 177029509 | 177029683 | surgical | hypermethylated |
| chr2 | 192113945 | 192114079 | surgical | hypermethylated |
| chr2 | 200326645 | 200326782 | surgical | hypermethylated |
| chr2 | 208989171 | 208989315 | surgical | hypermethylated |
| chr2 | 223161815 | 223161963 | surgical | hypermethylated |
| chr2 | 223162956 | 223163101 | surgical | hypermethylated |
| chr2 | 223163250 | 223163396 | surgical | hypermethylated |
| chr20 | 5282874 | 5283037 | surgical | hypomethylated |
| chr20 | 29979773 | 29979904 | surgical | hypomethylated |
| chr20 | 60119429 | 60119586 | surgical | hypomethylated |
| chr21 | 38076799 | 38076947 | surgical | hypermethylated |
| chr21 | 38076967 | 38077102 | surgical | hypermethylated |
| chr21 | 38077182 | 38077314 | surgical | hypermethylated |
| chr21 | 38082537 | 38082677 | surgical | hypermethylated |
| chr3 | 128202420 | 128202557 | surgical | hypermethylated |
| chr3 | 147106484 | 147106639 | surgical | hypermethylated |
| chr3 | 147108444 | 147108594 | surgical | hypermethylated |
| chr3 | 147108764 | 147108915 | surgical | hypermethylated |
| chr3 | 147109715 | 147109852 | surgical | hypermethylated |
| chr3 | 147113649 | 147113806 | surgical | hypermethylated |
| chr3 | 147113839 | 147113992 | surgical | hypermethylated |
| chr3 | 147127584 | 147127733 | surgical | hypermethylated |
| chr3 | 147128049 | 147128198 | surgical | hypermethylated |
| chr3 | 147131253 | 147131426 | surgical | hypermethylated |
| chr3 | 160167891 | 160168052 | surgical | hypermethylated |
| chr3 | 178907589 | 178907716 | surgical | hypermethylated |
| chr3 | 181421475 | 181421609 | surgical | hypermethylated |
| chr3 | 181421626 | 181421778 | surgical | hypermethylated |
| chr4 | 16639102 | 16639249 | surgical | hypomethylated |
| chr4 | 16773604 | 16773747 | surgical | hypomethylated |
| chr4 | 16795659 | 16795823 | surgical | hypomethylated |
| chr4 | 16862004 | 16862148 | surgical | hypomethylated |
| chr4 | 38871251 | 38871401 | surgical | hypermethylated |
| chr4 | 40336180 | 40336325 | surgical | hypomethylated |
| chr4 | 81189629 | 81189764 | surgical | hypermethylated |
| chr4 | 84469489 | 84469634 | surgical | hypomethylated |
| chr4 | 111550587 | 111550727 | surgical | hypermethylated |
| chr4 | 111550752 | 111550898 | surgical | hypermethylated |
| chr4 | 151504646 | 151504792 | surgical | hypermethylated |
| chr5 | 1879607 | 1879772 | surgical | hypermethylated |
| chr5 | 5146264 | 5146403 | surgical | hypomethylated |
| chr5 | 9782073 | 9782214 | surgical | hypomethylated |
| chr5 | 33737859 | 33738007 | surgical | hypomethylated |
| chr5 | 140174811 | 140174969 | surgical | hypermethylated |
| chr5 | 140810843 | 140810977 | surgical | hypermethylated |
| chr5 | 140811566 | 140811712 | surgical | hypermethylated |
| chr6 | 28227021 | 28227193 | surgical | hypermethylated |
| chr6 | 30130783 | 30131058 | surgical | hypomethylated |
| chr6 | 33141218 | 33141345 | surgical | hypomethylated |
| chr6 | 34984865 | 34985013 | surgical | hypermethylated |
| chr6 | 36253031 | 36253158 | surgical | hypermethylated |
| chr6 | 50791127 | 50791269 | surgical | hypermethylated |
| chr6 | 50813472 | 50813737 | surgical | hypermethylated |
| chr6 | 100905379 | 100905516 | surgical | hypermethylated |
| chr6 | 100912869 | 100913017 | surgical | hypermethylated |
| chr6 | 101846889 | 101847032 | surgical | hypermethylated |
| chr6 | 138866798 | 138866965 | surgical | hypermethylated |
| chr7 | 811109 | 811265 | surgical | hypermethylated |
| chr7 | 1596186 | 1596331 | surgical | hypomethylated |
| chr7 | 2609786 | 2609933 | surgical | hypermethylated |
| chr7 | 3988693 | 3988828 | surgical | hypermethylated |
| chr7 | 4786820 | 4787032 | surgical | hypermethylated |
| chr7 | 7759144 | 7759281 | surgical | hypomethylated |
| chr7 | 27142023 | 27142169 | surgical | hypermethylated |
| chr7 | 27204708 | 27204859 | surgical | hypermethylated |
| chr7 | 27204903 | 27205058 | surgical | hypermethylated |
| chr7 | 54612258 | 54612404 | surgical | hypermethylated |
| chr7 | 65617286 | 65617424 | surgical | hypermethylated |
| chr7 | 96621248 | 96621396 | surgical | hypermethylated |
| chr7 | 96622543 | 96622774 | surgical | hypermethylated |
| chr7 | 154087897 | 154088047 | surgical | hypomethylated |
| chr7 | 154428954 | 154429110 | surgical | hypomethylated |
| chr8 | 12236159 | 12236321 | surgical | hypomethylated |
| chr8 | 24151806 | 24151954 | surgical | hypomethylated |
| chr8 | 70981967 | 70982102 | surgical | hypermethylated |
| chr8 | 128807993 | 128808124 | surgical | hypomethylated |
| chr8 | 133072190 | 133072337 | surgical | hypomethylated |
| chr9 | 37002618 | 37002762 | surgical | hypermethylated |
| chr1 | 6165201 | 6165361 | cfDNA | hypomethylated |
| chr1 | 17567892 | 17568189 | cfDNA | hypomethylated |
| chr1 | 15426262 | 15426418 | cfDNA | hypomethylated |
| chr1 | 15670403 | 15670539 | cfDNA | hypermethylated |
| chr10 | 96279972 | 96280055 | cfDNA | hypomethylated |
| chr10 | 97033594 | 97033733 | cfDNA | hypermethylated |
| chr11 | 134245966 | 134246129 | cfDNA | hypermethylated |
| chr12 | 8004422 | 8004573 | cfDNA | hypermethylated |
| chr12 | 97140774 | 97140905 | cfDNA | hypermethylated |
| chr12 | 111566555 | 111566698 | cfDNA | hypermethylated |
| chr12 | 117750775 | 117750937 | cfDNA | hypermethylated |
| chr13 | 36828740 | 36828902 | cfDNA | hypermethylated |
| chr14 | 93214072 | 93214242 | cfDNA | hypomethylated |
| chr15 | 56006471 | 56006552 | cfDNA | hypermethylated |
| chr15 | 101547384 | 101547527 | cfDNA | hypomethylated |
| chr16 | 4141795 | 4141956 | cfDNA | hypermethylated |
| chr18 | 21857621 | 21857750 | cfDNA | hypomethylated |
| chr18 | 29528340 | 29528468 | cfDNA | hypermethylated |
| chr18 | 46845901 | 46846043 | cfDNA | hypermethylated |
| chr19 | 874766 | 874934 | cfDNA | hypomethylated |
| chr19 | 6799968 | 6800095 | cfDNA | hypomethylated |
| chr2 | 1126410 | 1126557 | cfDNA | differentially |
| chr2 | 225642009 | 225642217 | cfDNA | differentially |
| chr2 | 236745514 | 236745688 | cfDNA | hypomethylated |
| chr2 | 240881986 | 240882138 | cfDNA | differentially |
| chr2 | 2179742 | 2179886 | cfDNA | hypermethylated |
| chr2 | 30747398 | 30747539 | cfDNA | hypermethylated |
| chr2 | 175998270 | 175998415 | cfDNA | hypermethylated |
| chr2 | 219647407 | 219647560 | cfDNA | hypomethylated |
| chr20 | 20243607 | 20243747 | cfDNA | hypermethylated |
| chr20 | 55079800 | 55079945 | cfDNA | hypermethylated |
| chr21 | 30502729 | 30502871 | cfDNA | hypermethylated |
| chr21 | 46587906 | 46588052 | cfDNA | hypomethylated |
| chr3 | 56445240 | 56445378 | cfDNA | hypermethylated |
| chr3 | 85143433 | 85143600 | cfDNA | hypermethylated |
| chr3 | 146123966 | 146124095 | cfDNA | hypomethylated |
| chr3 | 68947379 | 68947542 | cfDNA | hypermethylated |
| chr3 | 197767819 | 197767978 | cfDNA | hypermethylated |
| chr4 | 143487129 | 143487273 | cfDNA | hypermethylated |
| chr4 | 26398190 | 26398329 | cfDNA | hypermethylated |
| chr4 | 77647893 | 77648027 | cfDNA | hypermethylated |
| chr4 | 102497551 | 102497732 | cfDNA | hypomethylated |
| chr5 | 39187156 | 39187287 | cfDNA | hypermethylated |
| chr5 | 56145736 | 56145896 | cfDNA | hypermethylated |
| chr5 | 160171748 | 160171896 | cfDNA | hypermethylated |
| chr5 | 16793080 | 16793219 | cfDNA | hypermethylated |
| chr5 | 76869108 | 76869253 | cfDNA | hypermethylated |
| chr6 | 169050287 | 169050447 | cfDNA | hypermethylated |
| chr6 | 76773251 | 76773422 | cfDNA | hypomethylated |
| chr6 | 123869831 | 123869971 | cfDNA | hypomethylated |
| chr7 | 6268960 | 6269087 | cfDNA | hypermethylated |
| chr7 | 38508407 | 38508486 | cfDNA | hypermethylated |
| chr7 | 153743779 | 153743947 | cfDNA | hypomethylated |
| chr7 | 137230794 | 137230963 | cfDNA | hypomethylated |
| chr7 | 151300131 | 151300282 | cfDNA | hypermethylated |
| chr8 | 3672236 | 3672387 | cfDNA | hypermethylated |
| chr8 | 99510084 | 99510252 | cfDNA | hypermethylated |
| chr8 | 101170822 | 101170975 | cfDNA | hypomethylated |
| chr8 | 141127042 | 141127183 | cfDNA | hypomethylated |
| chr9 | 2050654 | 2050804 | cfDNA | hypermethylated |
| chr9 | 9227683 | 9227824 | cfDNA | hypermethylated |
| chr9 | 79060522 | 79060633 | cfDNA | hypermethylated |
| chr9 | 124334690 | 124334848 | cfDNA | hypomethylated |
| chr9 | 126166694 | 126166828 | cfDNA | hypermethylated |
| chr1 | 180202441 | 180202578 | bChrSt | hypermethylated |
| chr10 | 102984159 | 102984316 | bChrSt | hypermethylated |
| chr10 | 102986926 | 102987078 | bChrSt | hypomethylated |
| chr10 | 124905661 | 124905811 | bChrSt | hypermethylated |
| chr11 | 18416284 | 18416422 | bChrSt | hypomethylated |
| chr11 | 20178032 | 20178171 | bChrSt | hypermethylated |
| chr11 | 20181732 | 20181875 | bChrSt | hypermethylated |
| chr11 | 31821190 | 31821332 | bChrSt | hypermethylated |
| chr11 | 31831813 | 31831955 | bChrSt | hypermethylated |
| chr12 | 6644024 | 6644165 | bChrSt | hypomethylated |
| chr13 | 100621076 | 100621217 | bChrSt | hypomethylated |
| chr13 | 100624236 | 100624376 | bChrSt | hypomethylated |
| chr14 | 23790611 | 23790772 | bChrSt | hypermethylated |
| chr17 | 46674335 | 46674487 | bChrSt | hypermethylated |
| chr17 | 48048913 | 48049070 | bChrSt | hypermethylated |
| chr2 | 162283732 | 162283879 | bChrSt | hypermethylated |
| chr2 | 175199619 | 175199764 | bChrSt | hypermethylated |
| chr2 | 175200596 | 175200742 | bChrSt | hypermethylated |
| chr2 | 223163736 | 223163879 | bChrSt | hypermethylated |
| chr4 | 13525615 | 13525755 | bChrSt | hypomethylated |
| chr4 | 113432474 | 113432622 | bChrSt | hypermethylated |
| chr6 | 100051116 | 100051256 | bChrSt | hypomethylated |
| chr6 | 100054673 | 100054827 | bChrSt | hypomethylated |
| chr6 | 100060971 | 100061117 | bChrSt | hypomethylated |

TABLE: 1B

Entity: Adenocarcinoma or squamous cell carcinoma?

| | | | | Entity |
|---|---|---|---|---|
| Chromosome | Start | End | Method | Meth. entities |
| chr1 | 52158087 | 52158220 | cfDNA, surgical | SQC<ADC |
| chr1 | 61668739 | 61668922 | cfDNA, surgical | SQC<ADC |
| chr1 | 64578151 | 64578293 | cfDNA, surgical | SQC<ADC |
| chr1 | 77533495 | 77533671 | cfDNA, surgical | SQC<ADC |
| chr1 | 171868017 | 171868187 | cfDNA, surgical | SQC<ADC |
| chr1 | 214646125 | 214646279 | cfDNA, surgical | SQC<ADC |
| chr11 | 1328403 | 1328548 | cfDNA, surgical | SQC<ADC |
| chr11 | 4079459 | 4079623 | cfDNA, surgical | SQC<ADC |
| chr11 | 71188639 | 71188789 | cfDNA, surgical | SQC<ADC |
| chr11 | 104972062 | 104972193 | cfDNA, surgical | SQC<ADC |
| chr11 | 105010212 | 105010354 | cfDNA, surgical | SQC<ADC |
| chr12 | 52946925 | 52947067 | cfDNA, surgical | SQC>ADC |
| chr12 | 88538122 | 88538272 | cfDNA, surgical | SQC<ADC |
| chr12 | 109096126 | 109096269 | cfDNA, surgical | SQC<ADC |
| chr14 | 90083196 | 90083338 | cfDNA, surgical | SQC<ADC |
| chr16 | 58155114 | 58155256 | cfDNA, surgical | SQC<ADC |
| chr17 | 29667240 | 29667387 | cfDNA, surgical | SQC<ADC |
| chr2 | 9987364 | 9987518 | cfDNA, surgical | SQC<ADC |
| chr2 | 25501964 | 25502121 | cfDNA, surgical | SQC<ADC |
| chr2 | 172266609 | 172266746 | cfDNA, surgical | SQC<ADC |
| chr2 | 178178843 | 178179018 | cfDNA, surgical | SQC<ADC |
| chr2 | 179897218 | 179897356 | cfDNA, surgical | SQC<ADC |
| chr20 | 31446106 | 31446254 | cfDNA, surgical | SQC<ADC |
| chr3 | 4348279 | 4348416 | cfDNA, surgical | SQC<ADC |
| chr3 | 38567580 | 38567725 | cfDNA, surgical | SQC<ADC |
| chr3 | 111629808 | 111629952 | cfDNA, surgical | SQC<ADC |
| chr3 | 114074222 | 114074369 | cfDNA, surgical | SQC<ADC |
| chr3 | 122841556 | 122841705 | cfDNA, surgical | SQC<ADC |
| chr3 | 150948199 | 150948350 | cfDNA, surgical | SQC<ADC |
| chr3 | 164915101 | 164915268 | cfDNA, surgical | SQC<ADC |
| chr5 | 122506718 | 122506853 | cfDNA, surgical | SQC<ADC |
| chr6 | 63990944 | 63991095 | cfDNA, surgical | SQC<ADC |
| chr6 | 64572767 | 64572911 | cfDNA, surgical | SQC<ADC |
| chr7 | 20381014 | 20381160 | cfDNA, surgical | SQC<ADC |
| chr7 | 21813010 | 21813162 | cfDNA, surgical | SQC<ADC |
| chr7 | 98722395 | 98722537 | cfDNA, surgical | SQC>ADC |
| chr7 | 102574027 | 102574188 | cfDNA, surgical | SQC<ADC |
| chr7 | 102574397 | 102574549 | cfDNA, surgical | SQC<ADC |
| chr7 | 111825737 | 111825894 | cfDNA, surgical | SQC<ADC |
| chr7 | 116377388 | 116377530 | cfDNA, surgical | SQC>ADC |
| chr7 | 122056569 | 122056711 | cfDNA, surgical | SQC<ADC |
| chr8 | 38643499 | 38643633 | cfDNA, surgical | SQC<ADC |
| chr8 | 42772303 | 42772478 | cfDNA, surgical | SQC>ADC |
| chr8 | 145599209 | 145599355 | cfDNA, surgical | SQC<ADC |
| chr1 | 3607047 | 3607181 | surgical | SQC<ADC |
| chr1 | 220101648 | 220101795 | surgical | SQC<ADC |
| chr1 | 220101867 | 220102015 | surgical | SQC<ADC |
| chr1 | 236849398 | 236849548 | surgical | SQC<ADC |
| chr1 | 236849891 | 236850048 | surgical | SQC<ADC |
| chr10 | 11206799 | 11206938 | surgical | SQC<ADC |
| chr11 | 30606998 | 30607133 | surgical | SQC<ADC |
| chr11 | 64992997 | 64993132 | surgical | SQC>ADC |
| chr11 | 64993266 | 64993396 | surgical | SQC>ADC |
| chr11 | 65360248 | 65360394 | surgical | SQC<ADC |
| chr11 | 77160268 | 77160416 | surgical | SQC<ADC |
| chr11 | 82444721 | 82444866 | surgical | SQC<ADC |
| chr12 | 4381723 | 4381963 | surgical | SQC<ADC |
| chr12 | 33592568 | 33592710 | surgical | SQC<ADC |
| chr13 | 28674372 | 28674520 | surgical | SQC<ADC |
| chr15 | 69087740 | 69087878 | surgical | SQC<ADC |
| chr15 | 83316148 | 83316297 | surgical | SQC<ADC |
| chr16 | 1202369 | 1202544 | surgical | SQC<ADC |
| chr16 | 56224714 | 56224858 | surgical | SQC<ADC |
| chr16 | 81564475 | 81564626 | surgical | SQC<ADC |
| chr16 | 86600252 | 86600386 | surgical | SQC<ADC |
| chr17 | 693067 | 693222 | surgical | SQC<ADC |
| chr17 | 693313 | 693458 | surgical | SQC<ADC |
| chr17 | 66292297 | 66292442 | surgical | SQC<ADC |
| chr17 | 74696666 | 74696814 | surgical | SQC<ADC |
| chr17 | 75196873 | 75197007 | surgical | SQC<ADC |
| chr17 | 80794200 | 80794346 | surgical | SQC<ADC |
| chr18 | 2847458 | 2847590 | surgical | SQC<ADC |
| chr18 | 24131050 | 24131188 | surgical | SQC<ADC |
| chr18 | 24131310 | 24131449 | surgical | SQC<ADC |
| chr19 | 10572284 | 10572428 | surgical | SQC<ADC |
| chr2 | 30834597 | 30834737 | surgical | SQC>ADC |
| chr2 | 50574632 | 50574774 | surgical | SQC<ADC |
| chr2 | 54054275 | 54054427 | surgical | SQC<ADC |
| chr2 | 63276135 | 63276276 | surgical | SQC<ADC |
| chr2 | 236444206 | 236444348 | surgical | SQC<ADC |
| chr20 | 20349092 | 20349238 | surgical | SQC<ADC |
| chr20 | 47444494 | 47444648 | surgical | SQC<ADC |
| chr20 | 47444775 | 47445083 | surgical | SQC<ADC |
| chr21 | 26934497 | 26934635 | surgical | SQC<ADC |
| chr3 | 141102520 | 141102668 | surgical | SQC<ADC |
| chr3 | 172167531 | 172167678 | surgical | SQC<ADC |
| chr3 | 172394613 | 172394766 | surgical | SQC<ADC |
| chr3 | 186914643 | 186914790 | surgical | SQC<ADC |
| chr3 | 196435366 | 196435518 | surgical | SQC<ADC |
| chr4 | 57522417 | 57522559 | surgical | SQC<ADC |
| chr4 | 57522562 | 57522846 | surgical | SQC<ADC |
| chr5 | 912634 | 912893 | surgical | SQC<ADC |
| chr5 | 1883876 | 1884018 | surgical | SQC<ADC |
| chr5 | 16179056 | 16179202 | surgical | SQC<ADC |
| chr5 | 33936177 | 33936331 | surgical | SQC<ADC |
| chr5 | 36607322 | 36607477 | surgical | SQC<ADC |
| chr5 | 169064355 | 169064518 | surgical | SQC<ADC |
| chr7 | 653234 | 653373 | surgical | SQC<ADC |
| chr7 | 1491753 | 1492006 | surgical | SQC<ADC |
| chr7 | 2158351 | 2158498 | surgical | SQC<ADC |
| chr7 | 4228700 | 4228842 | surgical | SQC<ADC |
| chr7 | 19156542 | 19156690 | surgical | SQC<ADC |
| chr7 | 19157127 | 19157340 | surgical | SQC<ADC |
| chr7 | 45197376 | 45197524 | surgical | SQC<ADC |
| chr8 | 41754102 | 41754249 | surgical | SQC<ADC |
| chr8 | 123874964 | 123875109 | surgical | SQC<ADC |
| chr8 | 123875144 | 123875280 | surgical | SQC<ADC |
| chr1 | 3289010 | 3289139 | cfDNA | SQC<ADC |
| chr1 | 17567892 | 17568189 | cfDNA | SQC>ADC |
| chr1 | 23284417 | 23284507 | cfDNA | SQC>ADC |
| chr1 | 24277975 | 24278154 | cfDNA | SQC>ADC |
| chr1 | 47738990 | 47739142 | cfDNA | SQC<ADC |
| chr1 | 79467955 | 79468081 | cfDNA | SQC>ADC |
| chr1 | 108975333 | 108975476 | cfDNA | SQC<ADC |
| chr1 | 196682870 | 196683025 | cfDNA | SQC<ADC |
| chr1 | 217310510 | 217310654 | cfDNA | SQC>ADC |
| chr1 | 240656480 | 240656649 | cfDNA | SQC<ADC |
| chr1 | 240746545 | 240746706 | cfDNA | SQC<ADC |
| chr1 | 246241918 | 246242056 | cfDNA | SQC<ADC |
| chr10 | 12533631 | 12533768 | cfDNA | SQC>ADC |
| chr10 | 32647546 | 32647656 | cfDNA | SQC>ADC |
| chr10 | 32657588 | 32657719 | cfDNA | SQC>ADC |
| chr10 | 37511104 | 37511239 | cfDNA | SQC>ADC |
| chr10 | 62708104 | 62708269 | cfDNA | SQC>ADC |
| chr10 | 73207931 | 73208064 | cfDNA | SQC<ADC |
| chr10 | 108812804 | 108812940 | cfDNA | SQC<ADC |
| chr10 | 115658133 | 115658275 | cfDNA | SQC>ADC |
| chr10 | 123914649 | 123914808 | cfDNA | SQC>ADC |
| chr11 | 15025357 | 15025499 | cfDNA | SQC>ADC |
| chr11 | 19778770 | 19778909 | cfDNA | SQC<ADC |
| chr11 | 26355535 | 26355711 | cfDNA | SQC>ADC |
| chr11 | 26600784 | 26600925 | cfDNA | SQC>ADC |
| chr11 | 26626367 | 26626558 | cfDNA | SQC>ADC |
| chr11 | 41275397 | 41275536 | cfDNA | SQC>ADC |
| chr11 | 62158845 | 62158985 | cfDNA | SQC>ADC |
| chr11 | 70503001 | 70503139 | cfDNA | SQC>ADC |
| chr11 | 106592142 | 106592304 | cfDNA | SQC<ADC |
| chr11 | 120644150 | 120644282 | cfDNA | SQC<ADC |
| chr11 | 122678508 | 122678636 | cfDNA | SQC<ADC |
| chr11 | 128851150 | 128851286 | cfDNA | SQC>ADC |
| chr12 | 125571801 | 125571933 | cfDNA | SQC>ADC |
| chr13 | 48806444 | 48806588 | cfDNA | SQC>ADC |
| chr13 | 113527733 | 113527876 | cfDNA | SQC<ADC |
| chr14 | 35030336 | 35030470 | cfDNA | SQC>ADC |
| chr14 | 104486171 | 104486314 | cfDNA | SQC>ADC |
| chr15 | 22839905 | 22840043 | cfDNA | SQC<ADC |
| chr15 | 26964926 | 26965065 | cfDNA | SQC>ADC |
| chr15 | 29246303 | 29246447 | cfDNA | SQC>ADC |
| chr15 | 30180680 | 30180842 | cfDNA | SQC<ADC |
| chr15 | 32404970 | 32405130 | cfDNA | SQC<ADC |
| chr15 | 64244033 | 64244215 | cfDNA | SQC<ADC |
| chr15 | 68530927 | 68531091 | cfDNA | SQC>ADC |
| chr15 | 83579367 | 83579513 | cfDNA | SQC<ADC |
| chr15 | 88559865 | 88560003 | cfDNA | SQC>ADC |
| chr16 | 6257325 | 6257474 | cfDNA | SQC>ADC |
| chr16 | 15665564 | 15665721 | cfDNA | SQC>ADC |
| chr16 | 24321180 | 24321320 | cfDNA | SQC<ADC |
| chr16 | 75528556 | 75528698 | cfDNA | SQC>ADC |
| chr16 | 88013993 | 88014135 | cfDNA | SQC<ADC |
| chr16 | 89713952 | 89714124 | cfDNA | SQC>ADC |
| chr17 | 416719 | 416865 | cfDNA | SQC<ADC |
| chr17 | 19809670 | 19809830 | cfDNA | SQC<ADC |
| chr17 | 21086965 | 21087112 | cfDNA | SQC>ADC |
| chr17 | 33364961 | 33365040 | cfDNA | SQC<ADC |
| chr17 | 64330485 | 64330837 | cfDNA | SQC>ADC |
| chr17 | 75142732 | 75142885 | cfDNA | SQC<ADC |
| chr19 | 11890923 | 11891074 | cfDNA | SQC<ADC |
| chr19 | 49016450 | 49016584 | cfDNA | SQC>ADC |
| chr19 | 57922060 | 57922195 | cfDNA | SQC>ADC |
| chr2 | 1129413 | 1129596 | cfDNA | SQC>ADC |
| chr2 | 1334513 | 1334640 | cfDNA | SQC>ADC |
| chr2 | 23917010 | 23917136 | cfDNA | SQC>ADC |
| chr2 | 25124037 | 25124165 | cfDNA | SQC>ADC |
| chr2 | 46779214 | 46779381 | cfDNA | SQC>ADC |
| chr2 | 113534514 | 113534653 | cfDNA | SQC<ADC |
| chr2 | 120417931 | 120418073 | cfDNA | SQC>ADC |
| chr2 | 131798797 | 131798977 | cfDNA | SQC<ADC |
| chr2 | 198073787 | 198073950 | cfDNA | SQC>ADC |
| chr2 | 205889570 | 205889704 | cfDNA | SQC>ADC |
| chr2 | 207319476 | 207319691 | cfDNA | SQC<ADC |
| chr20 | 9706282 | 9706429 | cfDNA | SQC>ADC |
| chr20 | 33713618 | 33713757 | cfDNA | SQC<ADC |
| chr21 | 33340955 | 33341038 | cfDNA | SQC<ADC |
| chr22 | 21206849 | 21206995 | cfDNA | SQC>ADC |
| chr22 | 30292326 | 30292475 | cfDNA | SQC<ADC |
| chr22 | 35697444 | 35697606 | cfDNA | SQC<ADC |
| chr3 | 3755582 | 3755730 | cfDNA | SQC>ADC |
| chr3 | 14959981 | 14960128 | cfDNA | SQC<ADC |
| chr3 | 25581721 | 25581859 | cfDNA | SQC>ADC |
| chr3 | 75834579 | 75834736 | cfDNA | SQC<ADC |
| chr3 | 87031909 | 87032079 | cfDNA | SQC<ADC |
| chr3 | 122710736 | 122710872 | cfDNA | SQC>ADC |
| chr3 | 139727561 | 139727706 | cfDNA | SQC<ADC |
| chr3 | 145864433 | 145864574 | cfDNA | SQC>ADC |
| chr4 | 1665996 | 1666155 | cfDNA | SQC>ADC |
| chr4 | 22518120 | 22518271 | cfDNA | SQC<ADC |
| chr4 | 77306769 | 77306948 | cfDNA | SQC<ADC |
| chr4 | 82520036 | 82520212 | cfDNA | SQC<ADC |
| chr4 | 155413871 | 155414011 | cfDNA | SQC<ADC |
| chr4 | 156601279 | 156601436 | cfDNA | SQC>ADC |
| chr4 | 162457724 | 162457860 | cfDNA | SQC>ADC |
| chr4 | 176636441 | 176636580 | cfDNA | SQC>ADC |
| chr4 | 177654193 | 177654363 | cfDNA | SQC<ADC |
| chr5 | 14450118 | 14450272 | cfDNA | SQC<ADC |
| chr5 | 75935318 | 75935450 | cfDNA | SQC>ADC |
| chr5 | 140475728 | 140475872 | cfDNA | SQC>ADC |
| chr5 | 146345906 | 146346062 | cfDNA | SQC>ADC |
| chr5 | 156458027 | 156458167 | cfDNA | SQC<ADC |
| chr5 | 157169890 | 157170038 | cfDNA | SQC>ADC |
| chr6 | 20832000 | 20832349 | cfDNA | SQC>ADC |
| chr6 | 24420281 | 24420413 | cfDNA | SQC<ADC |
| chr6 | 36331071 | 36331215 | cfDNA | SQC<ADC |
| chr6 | 54074847 | 54075021 | cfDNA | SQC>ADC |
| chr6 | 71122323 | 71122483 | cfDNA | SQC>ADC |
| chr6 | 83604672 | 83604779 | cfDNA | SQC<ADC |
| chr6 | 90709859 | 90710016 | cfDNA | SQC>ADC |
| chr6 | 111744738 | 111744881 | cfDNA | SQC>ADC |
| chr6 | 148806765 | 148806922 | cfDNA | SQC<ADC |
| chr6 | 155574119 | 155574263 | cfDNA | SQC<ADC |
| chr6 | 158460178 | 158460323 | cfDNA | SQC>ADC |
| chr7 | 5549605 | 5549675 | cfDNA | SQC<ADC |
| chr7 | 40669616 | 40669796 | cfDNA | SQC>ADC |
| chr7 | 73799798 | 73799908 | cfDNA | SQC>ADC |
| chr7 | 78030021 | 78030155 | cfDNA | SQC<ADC |
| chr7 | 81399230 | 81399365 | cfDNA | SQC<ADC |
| chr7 | 134452355 | 134452524 | cfDNA | SQC>ADC |
| chr7 | 140335200 | 140335344 | cfDNA | SQC>ADC |
| chr7 | 146925646 | 146925824 | cfDNA | SQC>ADC |
| chr7 | 153976496 | 153976643 | cfDNA | SQC>ADC |
| chr7 | 157941162 | 157941344 | cfDNA | SQC<ADC |
| chr7 | 157980130 | 157980264 | cfDNA | SQC<ADC |
| chr7 | 157980485 | 157980624 | cfDNA | SQC<ADC |
| chr7 | 158314155 | 158314301 | cfDNA | SQC<ADC |
| chr8 | 6392188 | 6392336 | cfDNA | SQC<ADC |
| chr8 | 11724061 | 11724159 | cfDNA | SQC<ADC |
| chr8 | 17237496 | 17237639 | cfDNA | SQC<ADC |
| chr8 | 21803649 | 21803801 | cfDNA | SQC<ADC |
| chr8 | 52696850 | 52697008 | cfDNA | SQC<ADC |
| chr8 | 72183950 | 72184120 | cfDNA | SQC>ADC |
| chr8 | 81042553 | 81042694 | cfDNA | SQC>ADC |
| chr8 | 85101824 | 85101952 | cfDNA | SQC>ADC |
| chr8 | 110703169 | 110703320 | cfDNA | SQC<ADC |
| chr8 | 121727803 | 121727944 | cfDNA | SQC<ADC |
| chr8 | 133476418 | 133476558 | cfDNA | SQC<ADC |
| chr9 | 8813022 | 8813150 | cfDNA | SQC<ADC |
| chr9 | 90258110 | 90258253 | cfDNA | SQC<ADC |
| chr9 | 97061691 | 97061835 | cfDNA | SQC>ADC |

TABLE 1C

Prognosis: favorable or unfavorable?

| Chromosome | Position | Method | Methylation |
|---|---|---|---|
| 1 | 18063105 | Paired biopsies (HM 450K) | high methylation = poor prognosis |
| 1 | 26699448 | Paired biopsies (HM 450K) | high methylation = poor prognosis |
| 1 | 115677211 | Paired biopsies (HM 450K) | high methylation = poor prognosis |
| 1 | 226187852 | Paired biopsies (HM 450K) | high methylation = poor prognosis |
| 1 | 226187876 | Paired biopsies (HM 450K) | high methylation = poor prognosis |
| 1 | 226188006 | Paired biopsies (HM 450K) | high methylation = poor prognosis |
| 2 | 27362420 | Paired biopsies (HM 450K) | high methylation = poor prognosis |
| 2 | 241314588 | Paired biopsies (HM 450K) | high methylation = poor prognosis |
| 2 | 241344707 | Paired biopsies (HM 450K) | high methylation = poor prognosis |
| 3 | 13914731 | Paired biopsies (HM 450K) | high methylation = poor prognosis |
| 5 | 176167283 | Paired biopsies (HM 450K) | high methylation = poor prognosis |
| 6 | 29528774 | Paired biopsies (HM 450K) | high methylation = poor prognosis |
| 6 | 154869909 | Paired biopsies (HM 450K) | high methylation = poor prognosis |
| 7 | 42195875 | Paired biopsies (HM 450K) | high methylation = poor prognosis |
| 8 | 10452896 | Paired biopsies (HM 450K) | high methylation = poor prognosis |
| 8 | 11614472 | Paired biopsies (HM 450K) | high methylation = poor prognosis |
| 8 | 49382369 | Paired biopsies (HM 450K) | high methylation = poor prognosis |
| 8 | 49466210 | Paired biopsies (HM 450K) | high methylation = poor prognosis |
| 8 | 49494724 | Paired biopsies (HM 450K) | high methylation = poor prognosis |
| 8 | 49496369 | Paired biopsies (HM 450K) | high methylation = poor prognosis |
| 8 | 49496391 | Paired biopsies (HM 450K) | high methylation = poor prognosis |
| 8 | 49533444 | Paired biopsies (HM 450K) | high methylation = poor prognosis |
| 8 | 49547126 | Paired biopsies (HM 450K) | high methylation = poor prognosis |
| 8 | 49823433 | Paired biopsies (HM 450K) | high methylation = poor prognosis |
| 9 | 133792985 | Paired biopsies (HM 450K) | high methylation = poor prognosis |
| 10 | 54223605 | Paired biopsies (HM 450K) | high methylation = poor prognosis |
| 11 | 1673436 | Paired biopsies (HM 450K) | high methylation = poor prognosis |
| 14 | 99700232 | Paired biopsies (HM 450K) | high methylation = poor prognosis |
| 20 | 584773 | Paired biopsies (HM 450K) | high methylation = poor prognosis |
| 20 | 2508981 | Paired biopsies (HM 450K) | high methylation = poor prognosis |
| 20 | 4201164 | Paired biopsies (HM 450K) | high methylation = poor prognosis |
| 20 | 43028501 | Paired biopsies (HM 450K) | high methylation = poor prognosis |
| 20 | 46323481 | Paired biopsies (HM 450K) | high methylation = poor prognosis |

TABLE 2

The kNN algorithm used ten positions to be able to distinguish the lung carcinoma patients from the healthy subjects. The column “Tumor” indicates whether an increased (+) or reduced (-) methylation was identified in tumor tissue

| a) ID | Chromosome | Position | Tumor |
|---|---|---|---|
| 596 | chr11 | 57006229 | + |
| 1717 | chr15 | 28262724 | + |
| 2636 | chr18 | 61144199 | - |
| 2805 | chr19 | 46823441 | |
| 4674 | chr2 | 176964685 | + |
| 4999 | chr2 | 225642035 | + |
| 5071 | chr3 | 14960020 | + |
| 5576 | chr4 | 13525705 | + |
| 6105 | chr5 | 140475760 | + |
| 6434 | chr6 | 46386723 | + |

| | | |
|---|---|---|
| b) Group | Accuracy | Number of samples |
| Malignant tumor | 1 | 9 |
| Control | 1 | 3 |
| Mean value | 1 | 12 |

Further parameters

| | |
|---|---|
| K | 5 |
| Ranking | Comparison of two group |
| Normalization | Mean value = 0, variance = 1 |
| Missing value | Mean value |

TABLE 3

The RT algorithm analyzed ten positions to ascertain the entity of a tumor. All positions were hypermethylated in the case of adenocarcinoma compared to squamous cell carcinoma

| a) ID | Chromosome | Position |
|---|---|---|
| 650 | chr11 | 64993331 |
| 2995 | chr1 | 17568007 |
| 4233 | chr2 | 50574690 |
| 4241 | chr2 | 50574708 |
| 4428 | chr2 | 111874494 |
| 4447 | chr2 | 121276804 |
| 5537 | chr4 | 1666074 |
| 5538 | chr4 | 1666075 |
| 6524 | chr6 | 83604790 |
| 7164 | chr7 | 69971740 |
| b) Group | Accuracy | Number of samples |
| Adenocarcinoma | 1 | 4 |
| Squamous cell carcinoma | 1 | 5 |
| Mean value | 1 | 6 |
| Further parameters | | |
| Max. depth | 25 | |
| Min. proportion of ranbom samples | 1% | |
| Max. number of categories | 10 | |
| Max. number of trees | 250 | |
| Accuracy of forest | 0.1 | |
| Criteria for termination | Max. number of trees | |
| Ranking | Comparison of two groups | |
| Normalization | Mean value = 0, variance = 1 | |
| Missing value | Mean value | |

TABLE 4

For staging (establishment of tumor stage), 523 positions were analyzed by the SVM algorithm. Some positions have increased methylation (+) in the late stage, while other positions have reduced methylation (-)

| a) ID | Chromosome | Position | Late (III, IV) stage |
|---|---|---|---|
| 16 | chr10 | 12533708 | + |
| 17 | chr10 | 12533710 | + |
| 20 | chr10 | 12533754 | - |
| 26 | chr10 | 15110983 | - |
| 37 | chr10 | 32657656 | - |
| 38 | chr10 | 32657672 | - |
| 79 | chr10 | 62708202 | + |
| 104 | chr10 | 97033706 | - |
| 123 | chr10 | 98129889 | - |
| 154 | chr10 | 102895057 | - |
| 164 | chr10 | 102984248 | + |
| 196 | chr10 | 102987003 | + |
| 199 | chr10 | 102987007 | + |
| 269 | chr10 | 121075316 | + |
| 281 | chr10 | 123914718 | - |
| 315 | chr10 | 124905781 | + |
| 320 | chr10 | 126494586 | - |
| 327 | chr10 | 126494644 | + |
| 333 | chr10 | 130860205 | - |
| 347 | chr10 | 134598357 | + |
| 349 | chr10 | 134598359 | + |
| 364 | chr11 | 627157 | - |
| 382 | chr11 | 1328455 | - |
| 385 | chr11 | 1328485 | - |
| 411 | chr11 | 15025433 | - |
| 431 | chr11 | 19778814 | + |
| 473 | chr11 | 26355627 | + |
| 479 | chr11 | 26626371 | + |
| 483 | chr11 | 26626471 | - |
| 554 | chr11 | 31831858 | + |
| 568 | chr11 | 31831899 | + |
| 572 | chr11 | 31831908 | + |
| 576 | chr11 | 31831919 | + |
| 585 | chr11 | 40136809 | + |
| 677 | chr11 | 69061863 | - |
| 696 | chr11 | 71188761 | - |
| 697 | chr11 | 71188762 | + |
| 709 | chr11 | 82444757 | - |
| 712 | chr11 | 82444771 | - |
| 742 | chr11 | 86085932 | + |
| 760 | chr11 | 113629710 | - |
| 767 | chr11 | 113629767 | - |
| 768 | chr11 | 114052115 | + |
| 793 | chr11 | 122678513 | + |
| 819 | chr11 | 134246113 | + |
| 822 | chr12 | 1943225 | + |
| 823 | chr12 | 1943226 | + |
| 824 | chr12 | 1943232 | + |
| 827 | chr12 | 2526357 | + |
| 831 | chr12 | 2526402 | + |
| 849 | chr12 | 2751699 | + |
| 867 | chr12 | 4381792 | - |
| 873 | chr12 | 4381812 | - |
| 880 | chr12 | 4381851 | + |
| 958 | chr12 | 33592641 | + |
| 963 | chr12 | 33592661 | + |
| 966 | chr12 | 33592673 | + |
| 970 | chr12 | 33592682 | + |
| 976 | chr12 | 34358517 | + |
| 1005 | chr12 | 34503613 | - |
| 1041 | chr12 | 54423543 | + |
| 1052 | chr12 | 54423567 | + |
| 1171 | chr12 | 97140890 | - |
| 1179 | chr12 | 108051521 | + |
| 1196 | chr12 | 112427829 | - |
| 1222 | chr12 | 123203607 | + |
| 1223 | chr12 | 123203612 | + |
| 1227 | chr12 | 123203644 | + |
| 1235 | chr12 | 125571833 | - |
| 1239 | chr12 | 126142895 | - |
| 1244 | chr12 | 129347679 | - |
| 1265 | chr12 | 129886069 | - |
| 1272 | chr12 | 129886183 | + |
| 1287 | chr13 | 36828832 | + |
| 1305 | chr13 | 48806483 | - |
| 1317 | chr13 | 58207814 | - |
| 1319 | chr13 | 58207831 | - |
| 1335 | chr13 | 93325573 | - |
| 1336 | chr13 | 93325602 | - |
| 1348 | chr13 | 100621150 | + |
| 1354 | chr13 | 100621175 | + |
| 1358 | chr13 | 100621185 | - |
| 1362 | chr13 | 100621194 | - |
| 1394 | chr13 | 100624346 | + |
| 1415 | chr14 | 23511181 | + |
| 1441 | chr14 | 35030414 | - |
| 1442 | chr14 | 35030415 | - |
| 1443 | chr14 | 35231195 | + |
| 1461 | chr14 | 37128597 | + |
| 1472 | chr14 | 55907282 | + |
| 1478 | chr14 | 55907299 | + |
| 1487 | chr14 | 57274719 | + |
| 1512 | chr14 | 57275127 | + |
| 1550 | chr14 | 57275995 | + |
| 1610 | chr14 | 57278179 | - |
| 1612 | chr14 | 57278187 | + |
| 1619 | chr14 | 57278220 | - |
| 1637 | chr14 | 58064926 | - |
| 1701 | chr14 | 104486258 | - |
| 1702 | chr14 | 104486260 | + |
| 1710 | chr15 | 26964987 | - |
| 1714 | chr15 | 28262702 | - |
| 1732 | chr15 | 32405064 | + |
| 1733 | chr15 | 32405065 | + |
| 1738 | chr15 | 41925179 | - |
| 1740 | chr15 | 41925187 | + |
| 1741 | chr15 | 41925188 | - |
| 1767 | chr15 | 45409283 | + |
| 1768 | chr15 | 45409293 | + |
| 1780 | chr15 | 56006548 | + |
| 1784 | chr15 | 63349191 | + |
| 1786 | chr15 | 63349194 | + |
| 1787 | chr15 | 63349195 | + |
| 1807 | chr15 | 69087782 | - |
| 1820 | chr15 | 72520614 | + |
| 1831 | chr15 | 83316217 | - |
| 1840 | chr15 | 83316257 | - |
| 1841 | chr15 | 83316258 | + |
| 1846 | chr15 | 83316269 | + |
| 1847 | chr15 | 83316270 | + |
| 1873 | chr15 | 89920814 | + |
| 1879 | chr15 | 89920855 | + |
| 1893 | chr15 | 89922297 | + |
| 1908 | chr15 | 89922344 | - |
| 1910 | chr15 | 89922358 | - |
| 1915 | chr15 | 89922387 | - |
| 1919 | chr15 | 98477887 | + |
| 1934 | chr16 | 526638 | + |
| 1956 | chr16 | 1202458 | - |
| 1973 | chr16 | 2880458 | + |
| 1974 | chr16 | 2880459 | + |
| 1991 | chr16 | 15665653 | + |
| 2014 | chr16 | 24822719 | - |
| 2056 | chr16 | 34255535 | - |
| 2067 | chr16 | 56224754 | + |
| 2071 | chr16 | 56224777 | + |
| 2080 | chr16 | 56224809 | - |
| 2106 | chr16 | 66613071 | + |
| 2133 | chr16 | 66613250 | + |
| 2157 | chr16 | 71528178 | + |
| 2171 | chr16 | 75528661 | + |
| 2187 | chr16 | 86600283 | + |
| 2199 | chr16 | 86600325 | + |
| 2205 | chr16 | 86600340 | + |
| 2214 | chr16 | 88014067 | + |
| 2216 | chr16 | 88014072 | + |
| 2218 | chr16 | 88014083 | - |
| 2222 | chr16 | 89714003 | + |
| 2225 | chr16 | 89714008 | + |
| 2226 | chr16 | 89714009 | - |
| 2237 | chr16 | 89714063 | + |
| 2249 | chr17 | 416795 | - |
| 2274 | chr17 | 750241 | - |
| 2285 | chr17 | 19809748 | - |
| 2286 | chr17 | 19809749 | - |
| 2291 | chr17 | 29174301 | + |
| 2299 | chr17 | 29174410 | + |
| 2317 | chr17 | 33314935 | - |
| 2322 | chr17 | 33314978 | - |
| 2327 | chr17 | 33314988 | + |
| 2333 | chr17 | 33365076 | - |
| 2356 | chr17 | 35299620 | + |
| 2371 | chr17 | 42960474 | + |
| 2373 | chr17 | 42960488 | + |
| 2389 | chr17 | 46799639 | + |
| 2391 | chr17 | 46799644 | + |
| 2393 | chr17 | 46799647 | - |
| 2394 | chr17 | 46799648 | + |
| 2406 | chr17 | 48048981 | + |
| 2411 | chr17 | 48049008 | + |
| 2435 | chr17 | 59532275 | + |
| 2443 | chr17 | 59532314 | + |
| 2453 | chr17 | 64330651 | + |
| 2459 | chr17 | 66292378 | - |
| 2463 | chr17 | 67536298 | - |
| 2465 | chr17 | 72619555 | - |
| 2481 | chr17 | 75142814 | + |
| 2506 | chr17 | 80794324 | - |
| 2530 | chr18 | 3971140 | + |
| 2541 | chr18 | 18658118 | - |
| 2545 | chr18 | 20714345 | + |
| 2550 | chr18 | 21596915 | + |
| 2559 | chr18 | 24131108 | - |
| 2574 | chr18 | 24131391 | + |
| 2600 | chr18 | 61143901 | + |
| 2624 | chr18 | 61144121 | - |
| 2665 | chr19 | 5141394 | + |
| 2688 | chr19 | 10572317 | - |
| 2711 | chr19 | 11891002 | - |
| 2739 | chr19 | 19625286 | - |
| 2756 | chr19 | 29991306 | - |
| 2767 | chr19 | 33102573 | + |
| 2769 | chr19 | 42600236 | + |
| 2789 | chr19 | 44629761 | + |
| 2791 | chr19 | 45782682 | + |
| 2803 | chr19 | 46823435 | - |
| 2815 | chr19 | 49016516 | - |
| 2820 | chr19 | 49016533 | - |
| 2824 | chr19 | 49503049 | + |
| 2839 | chr19 | 49909923 | - |
| 2862 | chr1 | 2198846 | + |
| 2863 | chr1 | 2198847 | + |
| 2867 | chr1 | 2198863 | + |
| 2958 | chr1 | 8787209 | + |
| 2962 | chr1 | 8787261 | - |
| 2973 | chr1 | 15426297 | + |
| 2984 | chr1 | 15670515 | - |
| 2992 | chr1 | 17568000 | - |
| 3005 | chr1 | 17568145 | + |
| 3017 | chr1 | 19764666 | + |
| 3019 | chr1 | 19764669 | + |
| 3025 | chr1 | 19764722 | - |
| 3031 | chr1 | 23284390 | - |
| 3033 | chr1 | 23284423 | - |
| 3045 | chr1 | 27234577 | - |
| 3053 | chr1 | 27234623 | - |
| 3056 | chr1 | 27234626 | - |
| 3066 | chr1 | 34642399 | + |
| 3110 | chr1 | 50883372 | + |
| 3121 | chr1 | 50886745 | + |
| 3127 | chr1 | 50886772 | + |
| 3164 | chr1 | 50886995 | + |
| 3174 | chr1 | 61668834 | + |
| 3176 | chr1 | 63489100 | - |
| 3225 | chr1 | 108975412 | - |
| 3227 | chr1 | 108975445 | + |
| 3284 | chr1 | 115677210 | + |
| 3315 | chr1 | 155162705 | - |
| 3348 | chr1 | 160783053 | - |
| 3390 | chr1 | 161008828 | + |
| 3392 | chr1 | 161008848 | + |
| 3402 | chr1 | 161306175 | - |
| 3454 | chr1 | 180202553 | - |
| 3488 | chr1 | 217310587 | + |
| 3501 | chr1 | 220101724 | + |
| 3509 | chr1 | 220101774 | - |
| 3519 | chr1 | 220101934 | + |
| 3538 | chr1 | 223948910 | + |
| 3554 | chr1 | 236849473 | + |
| 3579 | chr1 | 236849941 | - |
| 3586 | chr1 | 236849958 | + |
| 3616 | chr1 | 240656582 | + |
| 3630 | chr20 | 584741 | + |
| 3631 | chr20 | 584744 | + |
| 3636 | chr20 | 2508981 | - |
| 3641 | chr20 | 5282947 | - |
| 3651 | chr20 | 9706361 | + |
| 3652 | chr20 | 9706362 | + |
| 3655 | chr20 | 19910263 | + |
| 3724 | chr20 | 46323527 | + |
| 3748 | chr20 | 47444816 | + |
| 3762 | chr20 | 47444849 | + |
| 3764 | chr20 | 47444851 | + |
| 3795 | chr20 | 47444971 | + |
| 3809 | chr20 | 47445025 | - |
| 3923 | chr21 | 38077257 | + |
| 3970 | chr22 | 29956486 | + |
| 3977 | chr22 | 30292389 | - |
| 3990 | chr22 | 35697550 | + |
| 3992 | chr22 | 35697558 | + |
| 3999 | chr22 | 40810375 | + |
| 4005 | chr22 | 45129980 | + |
| 4018 | chr22 | 45992027 | + |
| 4021 | chr22 | 45992040 | - |
| 4070 | chr2 | 3642648 | + |
| 4078 | chr2 | 9987439 | - |
| 4142 | chr2 | 32313668 | + |
| 4158 | chr2 | 45171790 | + |
| 4183 | chr2 | 45232432 | + |
| 4236 | chr2 | 50574700 | - |
| 4257 | chr2 | 63276180 | - |
| 4258 | chr2 | 63276181 | - |
| 4265 | chr2 | 63276215 | + |
| 4299 | chr2 | 63281187 | + |
| 4391 | chr2 | 63284132 | + |
| 4399 | chr2 | 63284165 | - |
| 4406 | chr2 | 73021347 | - |
| 4414 | chr2 | 100209375 | - |
| 4434 | chr2 | 113534594 | + |
| 4446 | chr2 | 120418056 | + |
| 4472 | chr2 | 152992678 | - |
| 4479 | chr2 | 155089854 | - |
| 4550 | chr2 | 162283795 | + |
| 4574 | chr2 | 175199659 | + |
| 4597 | chr2 | 175199740 | + |
| 4642 | chr2 | 176964121 | + |
| 4683 | chr2 | 176964710 | + |
| 4761 | chr2 | 176988910 | + |
| 4768 | chr2 | 176988939 | + |
| 4797 | chr2 | 177014908 | + |
| 4800 | chr2 | 177014949 | + |
| 4820 | chr2 | 177027453 | - |
| 4845 | chr2 | 179897288 | + |
| 4864 | chr2 | 200326684 | + |
| 4873 | chr2 | 200326734 | + |
| 4998 | chr2 | 225642025 | + |
| 5005 | chr2 | 236444281 | + |
| 5023 | chr2 | 240882054 | + |
| 5030 | chr2 | 240882093 | + |
| 5040 | chr2 | 241314636 | + |
| 5054 | chr2 | 242273197 | + |
| 5057 | chr2 | 242273208 | - |
| 5058 | chr2 | 242273209 | - |
| 5059 | chr2 | 242273212 | + |
| 5072 | chr3 | 14960021 | + |
| 5093 | chr3 | 37544000 | - |
| 5097 | chr3 | 38567610 | + |
| 5104 | chr3 | 46904943 | + |
| 5108 | chr3 | 48837718 | + |
| 5120 | chr3 | 68947458 | + |
| 5122 | chr3 | 69280664 | + |
| 5125 | chr3 | 69280734 | - |
| 5146 | chr3 | 87032003 | + |
| 5147 | chr3 | 87032004 | + |
| 5153 | chr3 | 114074297 | + |
| 5156 | chr3 | 122710786 | + |
| 5157 | chr3 | 122710787 | + |
| 5160 | chr3 | 122710815 | + |
| 5162 | chr3 | 122710835 | + |
| 5163 | chr3 | 122710836 | + |
| 5164 | chr3 | 122710859 | + |
| 5165 | chr3 | 122710860 | + |
| 5167 | chr3 | 122841632 | - |
| 5282 | chr3 | 147108882 | + |
| 5303 | chr3 | 147113870 | + |
| 5380 | chr3 | 150948274 | - |
| 5388 | chr3 | 160167967 | + |
| 5391 | chr3 | 160167976 | + |
| 5436 | chr3 | 178907655 | + |
| 5483 | chr3 | 184742720 | + |
| 5510 | chr3 | 196440526 | - |
| 5522 | chr4 | 738427 | - |
| 5531 | chr4 | 738469 | - |
| 5532 | chr4 | 738470 | - |
| 5536 | chr4 | 1666071 | + |
| 5550 | chr4 | 8017839 | - |
| 5556 | chr4 | 8288935 | + |
| 5561 | chr4 | 13525657 | + |
| 5567 | chr4 | 13525666 | - |
| 5577 | chr4 | 13525721 | + |
| 5596 | chr4 | 21886957 | + |
| 5602 | chr4 | 26398259 | + |
| 5613 | chr4 | 57522468 | + |
| 5623 | chr4 | 57522505 | + |
| 5624 | chr4 | 57522506 | + |
| 5632 | chr4 | 57522618 | + |
| 5634 | chr4 | 57522620 | + |
| 5640 | chr4 | 57522642 | + |
| 5652 | chr4 | 57522762 | + |
| 5664 | chr4 | 71520485 | + |
| 5670 | chr4 | 77306910 | + |
| 5691 | chr4 | 82520132 | - |
| 5692 | chr4 | 82520133 | + |
| 5698 | chr4 | 94616253 | + |
| 5773 | chr4 | 113432594 | + |
| 5794 | chr4 | 151504732 | - |
| 5816 | chr4 | 176636539 | - |
| 5833 | chr4 | 183696160 | - |
| 5834 | chr4 | 183696161 | + |
| 5836 | chr4 | 183696165 | - |
| 5837 | chr4 | 186659550 | - |
| 5844 | chr5 | 912688 | + |
| 5861 | chr5 | 912783 | + |
| 5863 | chr5 | 912786 | + |
| 5865 | chr5 | 912803 | + |
| 5869 | chr5 | 912820 | + |
| 5872 | chr5 | 912834 | + |
| 5875 | chr5 | 912839 | + |
| 5947 | chr5 | 5146384 | - |
| 5949 | chr5 | 5568539 | + |
| 5955 | chr5 | 5568625 | - |
| 5956 | chr5 | 9782141 | - |
| 5975 | chr5 | 15824429 | - |
| 5992 | chr5 | 16179153 | + |
| 6040 | chr5 | 39187223 | + |
| 6065 | chr5 | 75935385 | + |
| 6081 | chr5 | 125881737 | + |
| 6083 | chr5 | 125881794 | - |
| 6115 | chr5 | 140475801 | + |
| 6128 | chr5 | 140810902 | - |
| 6130 | chr5 | 140810918 | + |
| 6156 | chr5 | 140811674 | - |
| 6163 | chr5 | 146345982 | - |
| 6164 | chr5 | 146345983 | - |
| 6167 | chr5 | 146346033 | - |
| 6169 | chr5 | 155481326 | + |
| 6174 | chr5 | 155481363 | + |
| 6186 | chr5 | 157169968 | - |
| 6191 | chr5 | 160171802 | + |
| 6194 | chr5 | 160171823 | + |
| 6206 | chr5 | 169064444 | - |
| 6233 | chr5 | 176167243 | - |
| 6237 | chr5 | 176167283 | + |
| 6253 | chr5 | 177966092 | - |
| 6255 | chr5 | 179180016 | + |
| 6273 | chr6 | 1656954 | - |
| 6274 | chr6 | 1656975 | + |
| 6281 | chr6 | 1657068 | + |
| 6283 | chr6 | 5132887 | + |
| 6302 | chr6 | 20832186 | - |
| 6309 | chr6 | 20832253 | - |
| 6363 | chr6 | 28227164 | - |
| 6365 | chr6 | 29528773 | - |
| 6367 | chr6 | 30130880 | + |
| 6368 | chr6 | 30130881 | + |
| 6390 | chr6 | 34984930 | - |
| 6394 | chr6 | 36253053 | + |
| 6502 | chr6 | 54074944 | + |
| 6511 | chr6 | 63991019 | - |
| 6512 | chr6 | 63991020 | + |
| 6531 | chr6 | 90709953 | - |
| 6537 | chr6 | 100051149 | + |
| 6565 | chr6 | 100054714 | + |
| 6616 | chr6 | 100061098 | + |
| 6625 | chr6 | 100905468 | + |
| 6666 | chr6 | 108675552 | - |
| 6674 | chr6 | 111744817 | - |
| 6693 | chr6 | 138866887 | + |
| 6698 | chr6 | 139205501 | + |
| 6723 | chr6 | 158056087 | - |
| 6760 | chr6 | 169050364 | + |
| 6778 | chr7 | 187685 | + |
| 6782 | chr7 | 187715 | + |
| 6784 | chr7 | 653308 | - |
| 6805 | chr7 | 1491812 | + |
| 6813 | chr7 | 1491843 | + |
| 6815 | chr7 | 1491858 | + |
| 6821 | chr7 | 1491902 | - |
| 6825 | chr7 | 1491921 | - |
| 6829 | chr7 | 1491966 | - |
| 6890 | chr7 | 4228760 | - |
| 6895 | chr7 | 4228818 | - |
| 6896 | chr7 | 4786867 | + |
| 6903 | chr7 | 4786899 | - |
| 6906 | chr7 | 4786942 | + |
| 6949 | chr7 | 6269027 | - |
| 6958 | chr7 | 19156576 | + |
| 6969 | chr7 | 19156620 | - |
| 6999 | chr7 | 19157240 | - |
| 7093 | chr7 | 40100597 | - |
| 7114 | chr7 | 44200085 | - |
| 7131 | chr7 | 45197448 | - |
| 7157 | chr7 | 65617361 | + |
| 7160 | chr7 | 65617365 | + |
| 7165 | chr7 | 69971769 | + |
| 7185 | chr7 | 73981010 | + |
| 7242 | chr7 | 96622713 | + |
| 7252 | chr7 | 102574104 | + |
| 7253 | chr7 | 102574105 | + |
| 7261 | chr7 | 102574475 | + |
| 7265 | chr7 | 102574499 | - |
| 7269 | chr7 | 102574518 | - |
| 7277 | chr7 | 111825813 | + |
| 7307 | chr7 | 134452411 | - |
| 7312 | chr7 | 134452451 | - |
| 7313 | chr7 | 134452452 | + |
| 7324 | chr7 | 140335252 | + |
| 7326 | chr7 | 140335261 | + |
| 7333 | chr7 | 141397873 | - |
| 7335 | chr7 | 146925681 | - |
| 7365 | chr7 | 151300170 | - |
| 7368 | chr7 | 151300199 | + |
| 7400 | chr7 | 154087938 | + |
| 7410 | chr7 | 157691297 | - |
| 7417 | chr7 | 157691343 | - |
| 7420 | chr7 | 157941187 | + |
| 7424 | chr7 | 157941301 | - |
| 7430 | chr7 | 157980193 | - |
| 7433 | chr7 | 157980216 | + |
| 7439 | chr7 | 158314202 | - |
| 7443 | chr7 | 158314235 | + |
| 7463 | chr8 | 6735007 | + |
| 7466 | chr8 | 10452854 | + |
| 7473 | chr8 | 10452918 | - |
| 7478 | chr8 | 11614472 | - |
| 7482 | chr8 | 11614502 | + |
| 7483 | chr8 | 11614519 | - |
| 7484 | chr8 | 11614520 | + |
| 7502 | chr8 | 14422449 | - |
| 7521 | chr8 | 26286645 | + |
| 7539 | chr8 | 31676500 | + |
| 7544 | chr8 | 31676524 | + |
| 7553 | chr8 | 31676686 | + |
| 7555 | chr8 | 31676697 | - |
| 7565 | chr8 | 41754146 | - |
| 7608 | chr8 | 49382365 | + |
| 7622 | chr8 | 49494676 | - |
| 7626 | chr8 | 49494699 | + |
| 7630 | chr8 | 49494723 | + |
| 7634 | chr8 | 49494773 | - |
| 7636 | chr8 | 49496331 | - |
| 7671 | chr8 | 49823388 | - |
| 7673 | chr8 | 49823395 | + |
| 7696 | chr8 | 62323244 | + |
| 7727 | chr8 | 72184078 | + |
| 7734 | chr8 | 85101900 | + |
| 7740 | chr8 | 110703242 | + |
| 7742 | chr8 | 110703245 | + |
| 7759 | chr8 | 123875007 | + |
| 7762 | chr8 | 123875033 | - |
| 7763 | chr8 | 123875034 | + |
| 7766 | chr8 | 123875051 | + |
| 7773 | chr8 | 123875079 | - |
| 7782 | chr8 | 123875253 | - |
| 7786 | chr8 | 126258311 | - |
| 7787 | chr8 | 126258312 | + |
| 7793 | chr8 | 129339255 | - |
| 7802 | chr8 | 141127120 | + |
| 7821 | chr9 | 8813092 | + |
| 7846 | chr9 | 37002714 | + |
| 7859 | chr9 | 74342704 | + |
| 7863 | chr9 | 74342751 | - |
| 7868 | chr9 | 79060545 | + |
| 7876 | chr9 | 90258167 | + |
| 7879 | chr9 | 90258174 | + |
| 7893 | chr9 | 97061769 | + |
| 7894 | chr9 | 97061770 | + |
| 7900 | chr9 | 115478948 | - |
| 7904 | chr9 | 115478954 | - |
| 7925 | chr9 | 126166763 | + |
| 7926 | chr9 | 126166764 | - |
| 7927 | chr9 | 133792936 | - |
| 7932 | chr9 | 133792984 | - |

| | | |
|---|---|---|
| b) Group | Accuracy | Number of samples |
| Early stage | 0 | 4 |
| Late stage | 0.8 | 5 |
| Mean value | 0.4 | 9 |

Further parameters

| | |
|---|---|
| Type | C_SVC |
| Kernel type | Linear |
| Output | 0.0001 |
| Nu | 0.5 |
| Epsilon SVR | 0.1 |
| Criteria for termination | Epsilon termination or max. iterations |
| Epsilon termination | 0.001 |
| Max. iterations | 1000 |
| Ranking | Comparison of two groups |
| Filtering according to | Group |
| Condition | Unequal, != |
| Normalization | Mean value = 0, variance = 1 |
| Missing value | Mean value |

TABLE 5

Exemplary oligonucleotides (capture targets), usable in the method according to the invention, for markers on Chromosome 1

| Start | Stop | | Length [bp] |
|---|---|---|---|
| 2198804 | 2198961 | chr1:2198830-2198930 | 157 |
| 3289010 | 3289139 | chr1:3289034-3289134 | 129 |
| 3607047 | 3607181 | chr1:3607067-3607167 | 134 |
| 6130197 | 6130338 | chr1:6130273-6130274 | 141 |
| 6165201 | 6165361 | chr1:6165229-6165329 | 160 |
| 6515521 | 6515702 | chr1:6515548-6515648;chr1:6515574-6515674 | 181 |
| 6520115 | 6520257 | chr1:6520145-6520245 | 142 |
| 8787128 | 8787253 | chr1:8787221-8787321,upstream | 125 |
| 15426262 | 15426418 | chr1:15426289-15426389 | 156 |
| 15670403 | 15670539 | chr1:15670433-15670533 | 136 |
| | | chr1: 17567922-17568022;chr1: 17568066- | |
| 17567892 | 17568189 | 17568166 | 297 |
| 18063027 | 18063184 | chr1:18063106-18063107 | 157 |
| 19177630 | 19177804 | chr1:19177728-19177729 | 174 |
| 19764609 | 19764757 | chr1:19764637-19764737 | 148 |
| 23284417 | 23284507 | chr1:23284374-23284474 | 90 |
| 24277975 | 24278154 | chr1:24278024-24278124 | 179 |
| 26699371 | 26699517 | chr1:26699448-26699449 | 146 |
| 27234664 | 27234812 | chr1:27234575-27234675,downstream | 148 |
| 34642324 | 34642455 | chr1:34642347-34642447 | 131 |
| 36194564 | 36194662 | chr1:36194581-36194582 | 98 |
| 38591827 | 38591977 | chr1:38591903-38591904 | 150 |
| 47694840 | 47694995 | chr1:47694870-47694970 | 155 |
| 47738990 | 47739142 | chr1:47739010-47739110 | 152 |
| 50883315 | 50883461 | chr1:50883345-50883445 | 146 |
| 50886707 | 50886857 | chr1:50886733-50886833 | 150 |
| 50886870 | 50887021 | chr1:50886900-50887000 | 151 |
| 52158087 | 52158220 | chr1:52158112-52158212 | 133 |
| 57955028 | 57955174 | chr1:57955057-57955157 | 146 |
| 61668739 | 61668922 | chr1:61668786-61668886 | 183 |
| 63489039 | 63489179 | chr1:63489116-63489117 | 140 |
| 64578151 | 64578293 | chr1:64578178-64578278 | 142 |
| 77533495 | 77533671 | chr1:77533543-77533643 | 176 |
| 79467955 | 79468081 | chr1:79467974-79468074 | 126 |
| 79472375 | 79472516 | chr1:79472403-79472503 | 141 |
| 85449266 | 85449364 | chr1:85449395-85449495,upstream | 98 |
| 108975333 | 108975476 | chr1:108975362-108975462 | 143 |
| 109383819 | 109383912 | chr1:109383701-109383801,downstream | 93 |
| 110610821 | 110610964 | chr1:110610850-110610950 | 143 |
| 110611386 | 110611542 | chr1:110611416-110611516 | 156 |
| 110611971 | 110612108 | chr1:110611995-110612095 | 137 |
| 115677141 | 115677297 | chr1:115677211-115677212 | 156 |
| 119522559 | 119522707 | chr1:119522588-119522688 | 148 |
| 150595130 | 150595282 | chr1:150595157-150595257 | 152 |
| 153896523 | 153896648 | chr1:153896541-153896641 | 125 |
| 154379671 | 154379808 | chr1:154379748-154379749 | 137 |
| 155162673 | 155162808 | chr1:155162703-155162803 | 135 |
| 158079244 | 158079395 | chr1:158079311-158079312 | 151 |
| 158324396 | 158324540 | chr1:158324422-158324522 | 144 |
| 158549201 | 158549351 | chr1:158549228-158549328 | 150 |
| 158575697 | 158575854 | chr1:158575724-158575824 | 157 |
| 158736216 | 158736378 | chr1:158736263-158736363 | 162 |
| 159284004 | 159284160 | chr1:159284033-159284133 | 156 |
| 159284209 | 159284363 | chr1:159284249-159284349 | 154 |
| 159682419 | 159682564 | chr1:159682448-159682548 | 145 |
| 160782978 | 160783141 | chr1:160783005-160783105 | 163 |
| | | chr1:161008656-161008756;chr1:161008701- | |
| 161008634 | 161008907 | 161008801;chr1:161008777-161008877 | 273 |
| 161284882 | 161285026 | chr1:161284950-161284951 | 144 |
| 161306252 | 161306382 | chr1:161306151-161306251,downstream | 130 |
| 166039366 | 166039510 | chr1:166039395-166039495 | 144 |
| 169138792 | 169138934 | chr1:169138868-169138869 | 142 |
| 170464175 | 170464329 | chr1:170464254-170464255 | 154 |
| 171868017 | 171868187 | chr1:171868066-171868166 | 170 |
| 175050401 | 175050549 | chr1:175050430-175050530 | 148 |
| 180202441 | 180202578 | chr1:180202463-180202563 | 137 |
| 182025968 | 182026117 | chr1:182025995-182026095 | 149 |
| 193191311 | 193191476 | chr1:193191356-193191456 | 165 |
| 196682870 | 196683025 | chr1:196682896-196682996 | 155 |
| 214646125 | 214646279 | chr1:214646154-214646254 | 154 |
| 217310510 | 217310654 | chr1:217310537-217310637 | 144 |
| 220101648 | 220101795 | chr1:220101678-220101778 | 147 |
| 220101867 | 220102015 | chr1:220101896-220101996 | 148 |
| 223948836 | 223948969 | chr1:223948861-223948961 | 133 |
| | | chr1:226187853-226187854;chr1:226187877- | |
| 226187776 | 226188068 | 226187878;chr1:226188006-226188007 | 292 |
| 236557105 | 236557253 | chr1:236557182-236557183 | 148 |
| 236849398 | 236849548 | chr1:236849424-236849524 | 150 |
| 236849891 | 236850048 | chr1:236849917-236850017 | 157 |
| 237765796 | 237765947 | chr1:237765826-237765926 | 151 |
| | | chr1:240656502-240656602;chr1:240656537- | |
| 240656480 | 240656649 | 240656637 | 169 |
| 240746545 | 240746706 | chr1:240746575-240746675 | 161 |
| 246241918 | 246242056 | chr1:246241939-246242039 | 138 |
| 248903024 | 248903175 | chr1:248903051-248903151 | 151 |

1. A method comprising determining the methylation of a set of methylation markers in a sample from a patient, wherein the set of methylation markers is selected from the group consisting of the regions listed in Table 1a, 1b and 1c and comprises at least 60 regions.

2. (canceled)

3. (canceled)

4. (canceled)

5. The method of claim 1, wherein the set of methylation markers comprises at least 60 regions selected from the group consisting of: chr1(6165201-6165361), chr1 (17567892-17568189), chr1 (15426262-15426418), chr115670403-15670539), chr2 (1126410-1126557), chr2 (225642009-225642217), chr2 (236745514-236745688), chr2 (240881986-240882138), chr2 (2179742-2179886), chr2 (30747398-30747539), chr2 (175998270-175998415), chr2 (219647407-219647560), chr3 (56445240-56445378), chr3 (85143433-85143600), chr3 (146123966-146124095), chr3 (68947379-68947542), chr3 (197767819-197767978), chr4 (143487129-143487273), chr4 (26398190-26398329), chr4 (77647893-77648027), chr4 (102497551-102497732), chr5 (39187156-39187287), chr5 (56145736-56145896), chr5 (160171748-160171896), chr5 (16793080-16793219), chr5 (76869108-76869253), chr6 (169050287-169050447), chr6 (76773251-76773422), chr6 (123869831-123869971), chr7 (6268960-6269087), chr7 (38508407-38508486), chr7 (153743779-153743947), chr7 (137230794-137230963), chr7 (151300131-151300282), chr8 (3672236-3672387), chr8 (99510084-99510252), chr8 (101170822-101170975), chr8 (141127042-141127183), chr9 (2050654-2050804), chr9 (9227683-9227824), chr9 (79060522-79060633), chr9 (124334690-124334848), chr9 (126166694-126166828), chr10 (96279972-96280055), chr10 (97033594-97033733), chr11 (134245966-134246129), chr12 (8004422-8004573), chr12 (97140774-97140905), chr12 (111566555-111566698), chr12 (117750775-117750937), chr13 (36828740-36828902), chr14 (93214072-93214242), chr15 (56006471-56006552), chr15 (101547384-101547527), chr16 (4141795-4141956), chr18 (21857621-21857750), chr18 (29528340-29528468), chr18 (46845901-46846043), chr19 (874766-874934), chr19 (6799968-6800095), chr20 (20243607-20243747), chr20 (55079800-55079945), chr21 (30502729-30502871), and chr21 (46587906-46588052)
wherein the presence of a tumor is analyzed, wherein the set of methylation markers optionally comprises all regions of the group.

6. The method of claim 5, wherein the set of methylation markers comprises at least 340 regions selected from the group consisting of the regions listed in Table 1a.

7. The method of claim 1, wherein the set of methylation markers comprises at least 134 regions selected from the group consisting of: chr1 (3289010-3289139, chr1 (17567892-17568189), chr1 (23284417-23284507), chr1 (24277975-24278154), chr1 (47738990-47739142), chr1 (79467955-79468081), chr1 (108975333-108975476), chr1 (196682870-196683025), chr1 (217310510-217310654), chr1 (240656480-240656649), chr1 (240746545-240746706), chr1 (246241918-246242056), chr2 (1129413-1129596), chr2 (1334513-1334640), chr2 (23917010-23917136), chr2 (25124037-25124165), chr2 (46779214-46779381), chr2 (113534514-113534653), chr2 (120417931-120418073), chr2 (131798797-131798977), chr2 (198073787-198073950), chr2 (205889570-205889704), chr2 (207319476-207319691), chr3 (3755582-3755730), chr3 (14959981-14960128), chr3 (25581721-25581859), chr3 (75834579-75834736), chr3 (87031909-87032079), chr3 (122710736-122710872), chr3 (139727561-139727706), chr3 (145864433-145864574), chr4 (1665996-1666155), chr4 (22518120-22518271), chr4 (77306769-77306948), chr4 (82520036-82520212), chr4 (155413871-155414011), chr4 (156601279-156601436), chr4 (162457724-162457860), chr4 (176636441-176636580), chr4 (177654193-177654363), chr5 (14450118-14450272), chr5 (75935318-75935450), chr5 (140475728-140475872), chr5 (146345906-146346062), chr5 (156458027-156458167), chr5 (157169890-157170038), chr6 (20832000-20832349), chr6 (24420281-24420413), chr6 (36331071-36331215), chr6 (54074847-54075021), chr6 (71122323-71122483), chr6 (83604672-83604779), chr6 (90709859-90710016), chr6 (111744738-111744881), chr6 (148806765-148806922), chr6 (155574119-155574263), chr6 (158460178-158460323), chr7 (5549605-5549675), chr7 (40669616-40669796), chr7 (73799798-73799908), chr7 (78030021-78030155), chr7 (81399230-81399365), chr7 (134452355-134452524), chr7 (140335200-140335344), chr7 (146925646-146925824), chr7 (153976496-153976643), chr7 (157941162-157941344), chr7 (157980130-157980264), chr7 (157980485-157980624), chr7 (158314155-158314301), chr8 (6392188-6392336), chr8 (11724061-11724159), chr8 (17237496-17237639), chr8 (21803649-21803801), chr8 (52696850-52697008), chr8 (72183950-72184120), , hr8 (81042553-81042694), chr8 (85101824-85101952), chr8 (110703169-110703320), chr8 (121727803-121727944), chr8 (133476418-133476558), chr9 (8813022-8813150), chr9 (90258110-90258253, chr9 (97061691-97061835), chr10 (12533631-12533768), chr10 (32657588-32657719), chr10 (37511104-37511239), chr10 (62708104-62708269), chr10 (73207931-73208064), chr10 (108812804-108812940), chr10 (115658133-115658275), chr10 (123914649-123914808), chr11 (15025357-15025499), chr11 (19778770-19778909), chr11 (26355535-26355711), chr11 (26600784-26600925), chr11 (26626367-26626558), chr11 (41275397-41275536), chr11 (62158845-62158985), chr11 (70503001-70503139), chr11 (106592142-106592304), chr11 (120644150-120644282), chr11 (122678508-122678636), chr11 (128851150-128851286), chr12 (125571801-125571933), chr13 (48806444-48806588), chr13 (113527733-113527876), chr14 (104486171-104486314), chr15 (22839905-22840043), chr15 (26964926-26965065), chr15 (29246303-29246447), chr15 (30180680-30180842), chr15 (32404970-32405130), chr15 (64244033-64244215), chr15 (68530927-68531091), chr15 (83579367-83579513), chr15 (88559865-88560003), chr16 (6257325-6257474), chr16 (15665564-15665721), chr16 (24321180-24321320), chr16 (75528556-75528698), chr16 (88013993-88014135), chr16 (89713952-89714124), chr17 (416719-416865), chr17 (19809670-19809830), chr17 (21086965-21087112), chr17 (33364961-33365040), chr17 (64330485-64330837), chr17 (75142732-75142885), chr19 (11890923-11891074), chr19 (49016450-49016584), chr19 (57922060-57922195), chr20 (9706282-9706429), chr20 (33713618-33713757), chr21 (33340955-33341038), chr22 (21206849-21206995), chr22 (30292326-30292475), and chr22 (35697444-35697606)
wherein the entity of a tumor is identified.

8. The method of claim 7, wherein the set of methylation markers comprises at least 240 regions, wherein the group consists of the regions listed in Table 1b.

9. (canceled)

10. A method comprising determining the methylation of a set of methylation markers in a sample from a patient, wherein the set of methylation markers comprises the following 10 positions: 596 (chr11, 57006229), 1717 (chr15, 28262724), 2636 (chr18, 61144199), 2805 (chr19, 46823441), 4674 (chr2, 176964685), 4999 (chr2, 225642035), 5071 (chr3, 14960020), 5576 (chr4, 13525705), 6105 (chr5, 140475760), and 6434 (chr6, 46386723).

11. A method determining the methylation of a set of methylation markers in a sample from a patient, wherein the set of methylation markers comprises the following 10 positions: 650 (chr11, 64993331), 2995 (chr1, 17568007), 4233 (chr2, 50574690), 4241 (chr2, 50574708), 4428 (chr2, 111874494), 4447 (chr2, 121276804), 5537 (chr4, 1666074), 5538 (chr4, 1666075), 6524 (chr6, 83604790), and 7164 (chr7, 69971740).

12. The method of claim 1, wherein the set of methylation markers comprises all positions listed in Table 4.

13. The method of claim 1, wherein the lung cancer is an NSCLC selected from the group comprising adenocarcinoma and squamous cell carcinoma, or an SCLC.

14. (canceled)

15. The method of claim 1, wherein the sample, is a liquid biopsy sample, or a solid tissue sample collected during surgery.

16. A method comprising obtaining a liquid biopsy sample from a subject, and determining the methylation of a set of methylation markers to obtain a cell-free DNA (cfDNA) methylation signature, wherein the set of methylation markers is selected from the group consisting of the regions listed in Table 1a, 1b and 1c and comprises at least 60 regions.

17. The method of claim 15, wherein the liquid biopsy sample is selected from the group comprising blood, plasma, serum, sputum, bronchial fluid and pleural effusion.

18. The method of claim 1, wherein the sample is a lung biopsy sample.

19. (canceled)

20. (canceled)

21. A means suitable for diagnosing lung cancer, wherein the means comprises oligonucleotides which can hybridize to DNA comprising methylation markers, wherein the set of methylation markers is selected from the group consisting of the regions listed in Table 1a, 1b and 1c and comprises at least 60 regions.

22. A method comprising:
a. extracting cfDNA from the liquid biopsy sample or genomic DNA from the
solid tissue sample,
b. carrying out a bisulfite conversion,
c. producing a whole-genome bisulfite sequencing library,
d. enriching DNA regions comprising the defined methylation markers, preferably comprising contacting them with the means of claim 21,
e. sequencing the enriched DNA regions,
f. aligning the sequencing data against a reference genome using the Segemehl algorithm, and
g. calculating the methylation rates.

23. (canceled)

24. A method for treating a lung tumor in a subject, comprising determining the methylation of a set of methylation markers in a sample from a patient according to the method of claim 1, detecting a pattern of methylation that identifies the presence, entity, stage, and/or prognosis of a lung tumor, and treating the subject with a suitable medicament, irradiation, or combination thereof.

25. The method of claim 24, comprising determining the entity of a lung tumor, selecting a therapy suitable for treatment of said entity, and treating the subject with the suitable medicament, irradiation, or combination thereof.

26. A method for treating a lung tumor in a subject, comprising determining the methylation of a set of methylation markers in a sample from a patient according to the method of claim 10, detecting a pattern of methylation that identifies the presence, entity, stage, and/or prognosis of a lung tumor, and treating the subject with a suitable medicament, irradiation, or combination thereof.

27. A method for treating a lung tumor in a subject, comprising determining the methylation of a set of methylation markers in a sample from a patient according to the method of claim 11, detecting a pattern of methylation that identifies the presence, entity, stage, and/or prognosis of a lung tumor, and treating the subject with a suitable medicament, irradiation, or combination thereof.
